(19) United States
(12) Patent Application Publication
(10) Pub. No.: US 2025/0025487 A1
(43) Pub. Date: Jan. 23, 2025

(54) THERAPEUTIC NUCLEIC ACIDS AND METHODS OF USE THEREOF

(71) Applicant: Cedars-Sinai Medical Center, Los Angeles, CA (US)

(72) Inventors: Eduardo Marbán, Santa Monica, CA (US); Ahmed G. Ibrahim, Los Angeles, CA (US)

(21) Appl. No.: 18/571,924

(22) PCT Filed: Jun. 30, 2022
(86) PCT No.: PCT/US2022/035866

§ 371 Date: Dec. 19, 2023

Related U.S. Application Data
(60) Provisional application No. 63/202,970, filed on Jul. 1, 2021.

(51) Int. Cl. A61K 31/712; A61P 9/04; A61P 9/10; A61P 21/00; C12N 15/113

(52) U.S. Cl. CPC A61K 31/712; A61P 9/04; A61P 9/10; A61P 21/00; C12N 15/113; C12N 2310/3231; C12N 2310/344

(57) ABSTRACT

An isolated nucleic acid that includes the nucleotide sequence: CGUCCGAUGGUAGUGGGUUAUCAG (SEQ ID NO: 12) is provided. In some embodiment, the nucleic acid includes at least one chemically-modified nucleotide. The nucleic acid and composition containing same find use in treating conditions associated with inflammation and/or fibrosis, for example, without limitation, heart conditions, such as hypertrophic myocardiopathy, myocardial infarction, and heart failure with preserved ejection fraction; muscle disorders, such as muscular dystrophy; skin disorders, such as scleroderma; and inflammatory conditions, such as autoimmune conditions or inflammatory conditions associated with a viral infection.

### CROSS REFERENCE TO RELATED APPLICATIONS

This application claims the benefit of U.S. Provisional Application No. 63/202,970, filed on Jul. 1, 2021, which is hereby incorporated by reference in its entirety.

### STATEMENT REGARDING FEDERALLY SPONSORED R&D

This invention was made with government support under Grant No. R01 HL124074, awarded to Dr. Eduardo Marbin by the National Institutes of Health. The Government has certain rights in the invention.

### REFERENCE TO SEQUENCE LISTING

The present application is being filed along with a Sequence Listing in electronic format. The Sequence Listing is provided as a file entitled CSMC018WO_SEQLIST.txt created on Jun. 30, 2022, which is 35,492 bytes in size. The information in the electronic format of the Sequence Listing is incorporated herein by reference in its entirety.

### BACKGROUND

### Field

The present disclosure relates to therapeutic RNA, variants thereof, and treatment of muscle and/or heart and/or inflammatory conditions using same.

Inflammation and tissue injury are main drivers of pathology in certain cardiac injury and other diseases. As crucial players in innate immunity, macrophages secrete inflammatory mediators, scavenge cellular debris (by efferocytosis), and remodel tissues after injury. Macrophages are key effectors of post-myocardial infarction (MI) cardioprotection induced by extracellular vesicles (EV) derived from cardiosphere-derived cells (CDC), and are implicated in enhanced efferocytosis. Consistent with this, macrophage depletion undermines cardioprotection.

### SUMMARY

Provided herein is an isolated nucleic acid comprising a nucleotide sequence of CGUCCGAUGGUAGUGGGUUAUCAG (SEQ ID NO: 12), wherein the nucleic acid is RNA, wherein the nucleic acid is at most 30 nt long. Also provided is an isolated nucleic acid comprising a nucleotide sequence at least 95% identical to CGUCCGAUGGUAGUGGGUUAUCAG (SEQ ID NO: 12), wherein the nucleic acid is RNA, wherein the nucleic acid is at most 30 nt long. Optionally, the nucleic acid comprises at least one chemically-modified nucleotide. In some embodiments, the nucleic acid comprises between 1-10 chemically-modified nucleotides. In some embodiments, the nucleic acid comprises at least one chemically-modified nucleotide within positions 1-12 and/or at least one chemically-modified nucleotide within positions 13-24 of the nucleotide sequence. In some embodiments, the chemically-modified nucleotide comprises a backbone modification. In some embodiments, the backbone modification comprises a backbone sugar modification. In some embodiments, the nucleic acid comprises the chemically-modified nucleotide at one or more of positions 1, 3, 5, 20, 22 and 24 of the nucleotide sequence. In some embodiments, the chemically-modified nucleotide is a locked nucleic acid (LNA). In some embodiments, the nucleotide sequence comprises the LNA at positions 1, 3, 5, 20, 22 and 24 of the nucleotide sequence. In some embodiments, the nucleic acid is 24 nucleotides long.

Provided herein is an isolated nucleic acid comprising a nucleotide sequence at least 95% identical to CGUCCGAUGGUAGUGGGUUAUCAG (SEQ ID NO: 12), wherein the nucleic acid is RNA. Optionally, the nucleotide sequence is CGUCCGAUGGUAGUGGGUUAUCAG (SEQ ID NO: 12). In some embodiments, the nucleic acid comprises at least one chemically-modified nucleotide. In some embodiments, the nucleic acid comprises between 1-10 chemically-modified nucleotides. In some embodiments, the nucleic acid comprises at least one chemically-modified nucleotide within positions 1-12 and/or at least one chemically-modified nucleotide within positions 13-24 of the nucleotide sequence. In some embodiments, the chemically-modified nucleotide comprises a backbone modification. In some embodiments, the backbone modification is a backbone sugar modification. In some embodiments, the nucleic acid further comprises the chemically-modified nucleotide at one or more of positions 1, 3, 5, 20, 22 and 24 of the nucleotide sequence. In some embodiments, the chemically-modified nucleotide is a locked nucleic acid (LNA). In some embodiments, the nucleotide sequence comprises the LNA at positions 1, 3, 5, 20, 22 and 24 of the nucleotide sequence. In some embodiments, the nucleic acid is at most 30 nt long.

In some embodiments, nucleic acid consists of or consists essentially of the nucleotide sequence: CGUCCGAUGGUAGUGGGUUAUCAG (SEQ ID NO: 12).

A nucleic acid consisting of a nucleotide sequence: CGUCCGAUGGUAGUGGGUUAUCAG (SEQ ID NO: 2), wherein the nucleic acid is RNA, wherein each of positions 1, 3, 5, 20, 22 and 24 of the nucleotide sequence is a LNA.

Also provided is a composition comprising: any one of the isolated nucleic acid of the present disclosure; and a pharmaceutically acceptable excipient. Optionally, the composition further comprises a transfection reagent. Optionally, the transfection reagent comprises one or more of a liposome, an extracellular vesicle (EV), and a polyethylene glycol (PEG)-cationic lipid complex (PCLC). In some embodiments, the transfection reagent comprises extracellular vesicles (EV) derived from cardiosphere-derived cells (CDC). In some embodiments, the composition further comprises a casein phosphoprotein. Optionally, the composition further comprises chitosan. Optionally, the isolated nucleic acid is encapsulated in a casein-chitosan complex. In some embodiments, the composition comprises casein micelles. In some embodiments, the composition comprises casein-chitosan micelles.

Provided herein is a macrophage comprising any one of the nucleic acids of the present disclosure, wherein an anti-inflammatory activity of the macrophage is increased compared to a macrophage without the nucleic acid. Also provided is a macrophage exposed to, or transfected with, any one of the nucleic acids of the present disclosure, wherein an anti-inflammatory activity of the macrophage is increased compared to a macrophage without the nucleic acid. Optionally, the macrophage is in a subject. Optionally, the macrophage is in culture.

Also provided is a kit comprising: any one of the nucleic acids of the present disclosure; and a transfection reagent. Optionally, the transfection reagent includes one or more of a lipid (e.g., a liposome-forming lipid), pegylated lipid, and an extracellular vesicle (EV). In some embodiments, the kit further comprises a pharmaceutically acceptable excipient. In some embodiments, the kit further comprises a casein phosphoprotein. In some embodiments, the kit further comprises chitosan.

Also provided is a method of treating a heart condition or symptom thereof, comprising administering to a subject in need of treating a heart condition or symptom thereof a therapeutically effective amount of any one of the nucleic acids of the present disclosure or any one of the compositions of the present disclosure, thereby treating the heart condition or symptom thereof. Optionally, the heart condition comprises a symptom and/or sequelae of heart failure. In some embodiments, the heart condition comprises hypertrophic cardiomyopathy. In some embodiments, the heart condition comprises heart failure with preserved ejection fraction (HFpEF). In some embodiments, the heart condition comprises a symptom or sequelae of an infectious disease. In some embodiments, the infectious disease comprises a viral infection. In some embodiments, the subject has the heart condition. In some embodiments, the subject is at risk of developing the heart condition. In some embodiments, the subject exhibits, before the administering, one or more of: hypertension, elevated E/e′ ratio by echocardiography, cardiac hypertrophy, myocardial fibrosis, obesity, wasting, reduced endurance, and elevated systemic inflammatory markers.

Also provided is a method treating a muscle disorder or symptom thereof, comprising administering to a subject in need of treating a muscle disorder or symptom thereof a therapeutically effective amount of any one of the nucleic acids of the present disclosure or any one of the compositions of the present disclosure, thereby treating the muscle disorder or symptom thereof. Optionally, the muscle disorder comprises muscular dystrophy or a heart condition. In some embodiments, the muscle disorder comprises Duchenne muscular dystrophy. In some embodiments, the subject has the muscle disorder. In some embodiments, the subject is at risk of developing the muscle disorder. In some embodiments, the subject is genetically predisposed to developing the muscle disorder. In some embodiments, the subject exhibits, before the administering, one or more of: reduced endurance, and reduced skeletal muscle function.

Also provided is a method of treating an inflammatory condition, comprising administering to a subject in need of treating an inflammatory condition a therapeutically effective amount of any one of the nucleic acids of the present disclosure or any one of the compositions of the present disclosure, thereby treating the inflammatory condition. Optionally, the inflammatory condition comprises a symptom or sequelae of an infectious disease. In some embodiments, the infectious disease comprises a viral infection. In some embodiments, the inflammatory condition comprises a cytokine storm. In some embodiments, the inflammatory condition is associated with immunotherapy (e.g., for cancer). In some embodiments, the inflammatory condition is scleroderma, an autoimmune condition affecting the skin. In some embodiments, the inflammatory condition is systemic sclerosis, an autoimmune condition resembling scleroderma but affecting not only the skin but also internal organs including the lung and the heart.

Also provided is a method of treating a fibrotic condition, comprising administering to a subject in need of treating a fibrotic condition a therapeutically effective amount of any one of the nucleic acids of the present disclosure or any one of the compositions of the present disclosure, thereby treating the fibrotic condition. Optionally, the fibrotic condition comprises a symptom or sequelae of an infectious disease. In some embodiments, the infectious disease comprises a viral infection. In some embodiments, the fibrotic condition is idiopathic pulmonary fibrosis. In some embodiments, the fibrotic condition is cirrhosis of the liver.

In some embodiments, the therapeutically effective amount of the nucleic acid comprises from about 0.001 μg/g to about 100 μg/g. In some embodiments, any one of the treatment methods of the present disclosure includes administering the therapeutically effective amount of the nucleic acid or the composition no more frequently than twice a week. In some embodiments, the method includes administering the therapeutically effective amount of the nucleic acid or the composition intravenously, intramuscularly, intracardially, or orally. Optionally, the therapeutically effective amount of the nucleic acid or the composition is administered orally.

Also provided is a method of promoting anti-inflammatory activity of macrophages, comprising contacting any one of the nucleic acids of the present disclosure or any one of the compositions of the present disclosure with a population of macrophages, to thereby promote an anti-inflammatory activity of macrophages of the population. Optionally, the contacting comprises administering to a subject in need of treating a condition characterized by inflammation and/or fibrosis an effective amount of the nucleic acid or the composition, to thereby promote an anti-inflammatory activity of macrophages in the subject. In some embodiments, the macrophage is a human macrophage.

Also provided is a method of treating a condition associated with inflammation and/or fibrosis, comprising administering to a subject in need of treating a condition associated with inflammation and/or fibrosis a therapeutically effective amount of a nucleic acid that binds Translocated Promoter Region (TPR), thereby treating the condition associated with inflammation and/or fibrosis. Optionally, the condition associated with inflammation and/or fibrosis comprises inflammation and/or fibrosis of the heart, skeletal muscle, or skin. In some embodiments, the condition associated with inflammation and/or fibrosis comprises a symptom and/or sequelae of heart failure, hypertrophic cardiomyopathy, heart failure with preserved ejection fraction (HFpEF), Duchenne muscular dystrophy, or scleroderma. In some embodiments, the nucleic acid that binds TPR inhibits TPR. In some embodiments, the nucleic acid that binds TPR reduces expression of TPR. In some embodiments, the nucleic acid that binds TPR comprises the nucleic acid of the present disclosure, e.g., TY4.

Also provided is a formulation for oral delivery of a nucleic acid, e.g., TY4, comprising the nucleic acid of the present disclosure, a cationic lipid, at least one casein protein, and a chitosan. In several embodiments, the nucleic acid of the formulation comprises TY4 or a derivative thereof, as provided herein, wherein the RNA is present in an amount ranging between 0.0001 and about 0.01% of the formulation by weight per volume. In several embodiments, the at least one casein protein comprises at least an α-s1 casein subunit that is present in an amount ranging between about 0.25 and about 7% of the formulation by weight per volume, the chitosan is present in an amount ranging between about 0.0001 and 4% of the formulation by weight per volume.

In several embodiments, there is provided a formulation for oral delivery of a nucleic acid, comprising a plurality of artificial lipid micelles, a plurality of nucleic acids, wherein a portion of the nucleic acids are encapsulated within the artificial lipid micelles, and a coating on the artificial lipid micelles, wherein the coating comprises a mixture of casein proteins and chitosan polymers. In several embodiments, the nucleic acid comprises a ribonucleic acid (RNA) and wherein the RNA is present in an amount ranging between about 0.00001 and about 0.05% of the formulation by weight per volume, the mixture of casein proteins and chitosan polymers comprises at least an α-s1 casein subunit that is present in an amount ranging between about 0.5 and about 5% of the formulation by weight per volume, and wherein the chitosan is present in an amount ranging between about 0.001 and about 1% of the formulation by weight per volume.

In several embodiments, the formulation further comprises an acid. In several embodiments, the acid is present in an amount ranging between about 0.001 and about 1% of the formulation by volume and the acid is selected from acetic acid, citric acid, phosphoric acid and citric acid. In one embodiment, the formulation further comprises acetic acid, wherein the acetic acid is present in an amount ranging between about 0.01 and about 1% of the formulation by weight per volume.

In several embodiments, the cationic lipid is present in an amount ranging from about 0.1 to about 5 microliters for each microgram of nucleic acid. In several embodiments, the chitosan is low molecular weight chitosan. In several embodiments, the low molecular weight chitosan ranges in mass from about 50 to about 190 kiloDaltons.

In several embodiments, the nucleic acid comprises a nucleic acid of the present disclosure, e.g., TY4, wherein the RNA is present in an amount ranging from between about 0.001 and about 0.005% of the formulation by weight per volume, wherein the at least one casein protein comprises a mixture of an α-s1 casein subunit, an α-s2 casein subunit, a β casein subunit, and a x casein subunit, wherein the casein subunits are present in an amount ranging between about 1 and 3% of the formulation by weight per volume, and wherein the chitosan is present in an amount ranging between about 0.01 and 0.1% of the formulation by weight per volume.

In several embodiments, the nucleic acid, e.g., TY4, is present in an amount ranging from between about 0.0015 and about 0.004% of the formulation by weight per volume, wherein the mixture of casein subunits are present in an amount ranging between about 2 and 3% of the formulation by weight per volume, wherein the chitosan is present in an amount ranging between about 0.05 and 0.1% of the formulation by weight per volume, and wherein the cationic lipid is present in an amount ranging from about 1 to about 3 microliters for each microgram of nucleic acid.

In several embodiments, the nucleic acid, e.g., TY4, is present in an amount ranging from between about 0.0015 and about 0.0035% of the formulation by weight per volume, wherein the mixture of casein subunits are present in an amount ranging between about 2.2 and 2.8% of the formulation by weight per volume, wherein the chitosan is present in an amount ranging between about 0.06 and 0.09% of the formulation by weight per volume, and wherein the cationic lipid is present in an amount ranging from about 1 to about 2 microliters for each microgram of nucleic acid.

### BRIEF DESCRIPTION OF THE DRAWINGS

FIG. 1A is a schematic diagram showing the EV-YF1 sequence and its hypothetical mechanism of action. FIG. 1B is a heat map image showing changes in gene expression in bone marrow-derived macrophages (BMDMs) treated with EV-YF1. FIG. 1C is graph showing differential expression of genes in BMDMs treated with EV-YF1. FIG. 1D is collection of graphs showing changes in histone acetylation pattern in cells treated with EV-YF1.

FIG. 2A is a schematic diagram showing a protocol for measuring the therapeutic effect of EV-YF1 in a model of hypertrophic cardiomyopathy (HCM). FIG. 2B is a graph showing change in body weight over time. FIG. 2C is a collection of echocardiogram images in diastole. FIG. 2D is a collection of graphs showing changes in diastolic interventricular septal wall diameter (IVSd; left panel) and left ventricular posterior wall diameter (LVPWd; right panel) over time. FIG. 2E is a collection of images comparing grooming behavior. FIG. 2F is a graph comparing treadmill exercise distances as percentage of distance run by WT mice. FIG. 2G is a collection of graphs comparing heart weight (HW)-to-tibial length (TL) ratio and relative lung weight (LW)-to-TL.

FIGS. 3A and 3B are collection of graphs showing comparison of expression levels of select genes in macrophages treated with a EV-YF1 variant or a vehicle control.

FIG. 4A is an image showing structural features of TY4 and its sequence alignment with EV-YF1. FIG. 4B is a heat map image showing changes in gene expression in BMDMs treated with TY4. FIG. 4C is a graph showing differential expression of genes in BMDMs treated with TY4.

FIGS. 5A-5E show the therapeutic effects of administering TY4 in hypertrophic cardiomyopathy (HCM), according to some non-limiting embodiments of the present disclosure. FIG. 5A is a schematic diagram showing a protocol for measuring the therapeutic effect of TY4 in a model of hypertrophic cardiomyopathy (HCM). FIG. 5B is a graphs comparing diastolic interventricular septal wall diameter (IVSd). FIG. 5C is a graph comparing left ventricular posterior wall diameter (LVPWd). FIG. 5D is a graph comparing treadmill exercise distances. FIG. 5E is a graph comparing changes in total exercise distance in a treadmill exercise test over time.

FIGS. 6A-6E show the therapeutic effects of administering TY4 in hypertrophic cardiomyopathy (HCM), according to some non-limiting embodiments of the present disclosure. FIG. 6A is a graph comparing systolic blood pressure. FIG. 6B is a graph comparing diastolic blood pressure. FIG. 6C is a graph comparing changes in systolic blood pressure over time during TY4 administration. FIG. 6D is a graph comparing changes in diastolic blood pressure over time. FIG. 6E is graph comparing changes in body weight over time.

FIGS. 7A-7C show the therapeutic effects of administering TY4 in hypertrophic cardiomyopathy (HCM), according to some non-limiting embodiments of the present disclosure. FIG. 7A is a graph comparing brain natriuretic peptide (BNP) levels in peripheral blood. FIG. 7B is a graph comparing heart weight (HW)-to-tibial length (TL) ratio. FIG. 7C is a graph comparing relative lung weight (LW)-to-TL.

FIGS. 8A-8M show the therapeutic effects of administering TY4 in heart failure with preserved ejection fraction (HFpEF), according to some non-limiting embodiments of the present disclosure. FIG. 8A is a schematic diagram showing a protocol for measuring the therapeutic effect of TY4 in a model of HFpEF. FIG. 8B is a graph comparing systolic blood pressure (left panel) and diastolic blood pressure (right panel).

FIG. 8C is a graph comparing treadmill exercise distances. FIG. 8D is a graph comparing the ratio of early diastolic mitral inflow velocity to mitral annular tissue velocity (E/e′). FIG. 8E is a graph comparing systemic brain natriuretic peptide (BNP) levels. FIG. 8F is a collection of graphs comparing systolic blood pressure (left panel) and diastolic blood pressure (right panel). FIG. 8G is a graphs comparing the ratio of early diastolic mitral inflow velocity to mitral annular tissue velocity (E/e′). FIG. 8H is a graph comparing treadmill exercise distances. FIG. 8I is a graph comparing systemic BNP levels. FIG. 8J is a graph comparing systolic blood pressure over time. FIG. 8K is a graph comparing diastolic blood pressure over time. FIG. 8L is a graph comparing E/e′ ratios over time. FIG. 8M is a graph comparing treadmill exercise distances over time.

FIGS. 9A-9G show the therapeutic effects of administering TY4 in HFpEF, according to some non-limiting embodiments of the present disclosure. FIG. 9A is a graph comparing E/e′ ratios. FIG. 9B is a graph comparing E/e′ ratios over time. FIG. 9C is a graph comparing body weight over time. FIG. 9D is a graph comparing treadmill exercise distances. FIG. 9E is a graph comparing treadmill exercise distances over time. FIG. 9F is a collection of graphs comparing systolic blood pressure (upper panel) and relative changes in systolic blood pressure over time (lower panel). FIG. 9G is a collection of graphs comparing diastolic blood pressure (upper panel) and relative changes in diastolic blood pressure over time (lower panel).

FIGS. 10A and 10B show the therapeutic effects of administering TY4 in HFpEF, according to some non-limiting embodiments of the present disclosure. FIG. 10A is a graph comparing interleukin (IL)-6 levels in peripheral blood, in pg/mL. FIG. 10B is a graph comparing BNP levels in peripheral blood, in pg/mL.

FIGS. 11A-11B shows determining biodistribution of CDC-EVs by measuring human-specific Y-RNA sequences, according to some non-limiting embodiments of the present disclosure. FIG. 11A is a sequence alignment of a human-specific Y-RNA sequence and the closest mouse sequence. FIG. 11B is a plot showing detection of human-specific Y-RNA sequence in tissue as a function of human CDC-EV amount in the tissue.

FIG. 11C is a graph comparing the measured amount of human CDC-EV in different tissues after retro-orbital injection of the CDC-EV.

FIG. 12A is a schematic diagram of a human macrophage screen for bioactive therapeutic molecules, according to some non-limiting embodiments of the present disclosure.

FIG. 12B is a graph showing expression of IL10 mRNA in human macrophages.

FIG. 13 is a schematic diagram showing an in vitro assay for testing injury-modifying bioactivity of TY4 and/or derivatives thereof, and alternative formulations and routes of administration for TY4 and/or derivative thereof, according to some non-limiting embodiments of the present disclosure.

FIG. 14A is a collection of schematic diagrams and plots showing a protocol for generating CDC-EV loaded with chemically modified RNA, according to some non-limiting embodiments of the present disclosure.

FIG. 14B is a collection of a schematic diagram, a plot and a graph showing size distribution and amount of exogenous RNA in CDC-EV loaded with chemically modified RNA.

FIG. 14C is a collection of a schematic diagram and images showing delivery of exogenous RNA to neonatal rat cardiomyocytes by CDC-EV loaded with chemically modified RNA.

FIG. 15 is a schematic diagram showing a protocol for measuring the therapeutic effects of administering TY4 in mdx mice, an established model of Duchenne muscular dystrophy.

FIGS. 16A-16D show the therapeutic effects on in vivo muscle function of administering TY4 in mdx mice, an established model of Duchenne muscular dystrophy), according to some non-limiting embodiments of the present disclosure. FIG. 16A is a graph showing treadmill exercise distances over time in vehicle control animals. FIG. 16B is a graph showing treadmill exercise distances over time in methylated EV-YF1 (Y RNAme) treated animals. FIG. 16C is a graph showing treadmill exercise distances over time in TY4-treated animals. FIG. 16D is a graph showing changes over time in treadmill exercise distances relative to baseline.

FIGS. 17A-17F show therapeutic effects on in vitro muscle function of administering TY4 in mdx mice, a model of Duchenne muscular dystrophy), according to some non-limiting embodiments of the present disclosure. FIG. 17A is a graph showing tetanic torque of skeletal muscle over time in vehicle control animals. FIG. 17B is a graph showing tetanic torque of skeletal muscle over time in methylated EV-YF1 (Y RNAme) treated animals. FIG. 17C is a graph showing tetanic torque of skeletal muscle over time in TY4-treated animals. FIG. 17D is a graph showing twitch torque of skeletal muscle over time in vehicle control animals. FIG. 17E is a graph showing twitch torque of skeletal muscle over time in methylated EV-YF1 (Y RNAme) treated animals. FIG. 17F is a graph showing twitch torque of skeletal muscle over time in TY4-treated animals.

FIG. 18A is a graph showing the change in tetanic torque relative to baseline over time.

FIG. 18B is a graph showing the magnitude of the therapeutic effect of EV-YF1 (Y RNAme) and TY4 on tetanic torque relative to control.

FIGS. 19A-19L are a collection of schematic diagrams, graphs and images showing that TY4 is an engineered Y-derived small RNA (YsRNA) with anti-senescent properties. FIG. 19A: TY4 was bioinspired from a larger Y-derived small RNA (EV-YF1) naturally abundant in the extracellular vesicles of therapeutic cardiac stromal cells (Cardiosphere-derived cells; CDCs). The truncated version is inspired by a shorter variant also abundant in CDC-EVs. TY4 contains a mutation (G to C) at the 5″ end and the inclusion of three alternating locked nucleic acid residues (LNA) on each end. FIG. 19B: Predicted structure of TY4 (spectrum represents base-pairing probability). FIG. 19C: Heatmap of bone marrow-derived macrophages (BMDM) exposed to vehicle (PBS) or TY4 (n=4 biological replicates per group). FIGS. 19D-19H: Sequencing data showing upregulation of immunoregulatory mediators (FIG. 19D), suppression of TGFβ pro-fibrotic signaling (FIG. 19E), and hypertrophic signaling (FIG. 19F) and their upstream potentiators Hippo/Yap (FIG. 19G) and MAPK signaling (FIG. 19H). FIGS. 19I-19L: BMDMs pre-treated with vehicle, TY4 and exposed to lipopolysaccharide showed reduced markers of senescence (p21; FIG. 19I), inflammasome activation (IL1b; FIG. 19J), and inflammation (NFkB and IL6; FIGS. 19K, 19L, respectively) compared to vehicle or a TY4 scrambled sequence. Analysis of histone modification further indicates that macrophages exposed to TY4 had differential changes in histone modification (n=3 biological replicates; FIG. 20D). Bars represent group mean and error bars represent s.d. Significance was determined by one-way ANOVA; *, ^{†}P<0.05; **, ^{††}P<0.01; ***, ^{†††}P<0.001. *; denotes comparison between groups and vehicle, †; denotes comparison between groups and control.

FIGS. 20A-20D are a collection of graphs showing that TY4 has anti-senescent properties. FIGS. 20A, 20B: Sequencing data showing suppression of pro-fibrotic a disintegrin and metalloprotease (ADAMs) and matrix metalloproteinase (MMPs) (FIG. 20A) and collagen (FIG. 20B) in TY4-treated macrophages compared to vehicle. FIG. 20C: Modulation of histone deacetylases and methyl transferases (Smyd and Prdm) in TY4-treated macrophages compared to control. FIG. 20D: Modified histone protein array showing changes in the methylation and acetylation profile of macrophages treated with TY4 compared to scramble control (represented by dotted line; n=1 sample per group). Bars represent group mean and error bars represent s.d. Significance was determined by Student's t-test; *P<0.05; **P<0.01; ***P<0.001.

FIGS. 21A-21E are a collection of schematic diagrams and graphs showing that TY4 administration is therapeutically bioactive in a mouse two-hit model of heart failure with preserved ejection fraction (HFpEF). FIG. 21A: Study outline for investigating the efficacy of TY4 in a mouse two-hit model of HFpEF. Wildtype c57BL/6 mice were placed on a high-fat diet and the nitric oxide synthase inhibitor No-Nitro-L-arginine methyl ester (L-NAME) for five weeks whereafter they were randomized to receive intravenous (retro-orbital) infusions of saline (negative control) or TY4 (0.15 μg/g) at frequencies of every other week, weekly, or biweekly (total of 10 weeks). FIGS. 21B-21E: TY4 administration reduced diastolic dysfunction (FIG. 21B; n=4-10 animals per group), improved exercise endurance (FIG. 21C; n=7-10 per group), and reduced circulating brain naturietic peptide (FIG. 21D; n=3-4 per group) and IL6 (FIG. 21E; n=3-4 animals per group). Bars and scatter plot lines represent group mean and error bars represent s.d. Significance was determined by one-way ANOVA; *, ^{†}P<0.05; **, ^{††}P<0.01; ***, ^{†††}P<0.001. *; denotes comparison between groups and vehicle, †; denotes comparison between groups and control.

FIGS. 22A-22G are a collection of graphs showing the effects of TY4 delivery in HFpEF mice. FIG. 22A: Relative abundance of TY4 in the liver, heart, and plasma of animals at 30 minutes post intravenous administration of TY4 compared to tissue from vehicle controls (n=3 animals per group). FIG. 22B: confirmation of conserved ejection fraction in HFpEF mice groups (n=3 per group). FIGS. 22C, 22D: attenuation of systolic and diastolic blood pressure (respectively) in HFpEF animals treated with TY4 compared to vehicle (n=5-10 animals per group). FIGS. 22E-22G: Loss of TY4 therapeutic bioactivity in HFpEF animals receiving TY4 without transfection reagent (−TR) including loss beneficial effects in diastolic dysfunction (FIG. 22E), exercise endurance (FIG. 22F), and circulating BNP levels (FIG. 22G) (n=4-5 per group). Bars and lines represent group mean and error bars represent s.d. Significance was determined by one-way ANOVA; *P<0.05; **P<0.01.

FIGS. 23A-23E are a collection of graphs showing that TY4 restores levels of HFpEF dysregulated genes in mice. Sequencing results showing restoration of HFpEF-dysregulated genes (compared to vehicle) including structural (FIG. 23A), developmental (FIG. 23B), calcium handing (FIG. 23C), potassium channels (FIG. 23D), and solute carrier membrane proteins (FIG. 23E) (n=5 hearts per group).

FIGS. 24A-24J are a collection of graphs and images showing that TY4 targets the p21-Smyd4 axis to attenuate cell stress and inflammation. FIG. 24A: Heatmap of HFpEF mouse hearts. FIGS. 24B-24E: TY4 suppresses cell stress signaling in diseased hearts (compared to vehicle) starting with p21-p16 senescent pathway (FIG. 24B), endoplasmic reticulum (ER) stress luminal receptors (FIG. 24C), inflammasome signaling (FIG. 24D), and downstream inflammatory markers (FIG. 24E) (n=5 animals per group). FIGS. 24F, 24G: P21 is suppressed in TY4-treated HFpEF mice as shown by gene expression (FIG. 24F), and protein (FIG. 24G, bar graph and Western blot images below) compared to control (n=3 hearts per group). FIGS. 24H-24J: Upregulation in gene expression (FIG. 24H), and protein levels (FIG. 24I), of the histone methyltransferase potentiator of p21, smyd4 (FIG. 24J) (n=3 per hearts per group). Bars and points represent group mean and error bars represent s.d. Significance was determined by one-way ANOVA; *P<0.05; **P<0.01.

FIGS. 25A-25H are a collection of graphs and images showing that TY4 suppresses P21 in tissue of HFpEF mice. FIGS. 25A-25D: QPCR demonstrating TY4 suppression of p21 in lung (FIG. 25A), liver (FIG. 25B), spleen (FIG. 25C), and kidney (FIG. 25D) tissue in HFpEF mice (compared to vehicle; n=3-5 animals per group). FIGS. 25E, 25F: suppression of protein levels of p21 in serum (n=3-5 animals per group) and tissue (n=3 animals per group). FIG. 25G: Sequencing data from HFpEF hearts identifying two histone methyltransferases Prdm2 and Smyd4 as suppressed by TY4 treatment. FIG. 25H: Protein levels of Prdm2 in the TY4-treated HFpEF hearts are unchanged compared to vehicle (n=4 animals per group). Bars and box plot midlines represent group mean and error bars represent s.d. Significance was determined by one-way ANOVA; *P<0.05; **P<0.01; ***P<0.001. *; denotes comparison between groups and vehicle, †; denotes comparison between groups and control.

FIGS. 26A-26D are a collection of schematic diagrams, graphs and images showing efficacy of TY4 in a model of myocardial infarction. FIG. 26A: a rat model of myocardial infarction. Animals receiving intravenous TY4 had reduced infarct size (FIGS. 26B, 26C) and lower levels of circulating cardiac troponin (FIG. 26D) compared to vehicle control or scramble.

FIGS. 27A and 27B are a collection of a schematic diagram and a graph showing that TY4 improves outcomes in a porcine model of myocardial infarction. FIG. 27A: a porcine model of myocardial infarction. Animals receiving intravenous TY4 had reduced infarct size (FIG. 27B) compared to vehicle control or scramble.

FIGS. 28A-28F are a collection of schematic diagrams, graphs and images showing that TY4 improves outcomes in a mouse model of Duchenne Muscular Dystrophy. FIG. 28A: Study design; Mdx animals received intravenous infusions of TY4 twice a week for 8 weeks. Animals receiving TY4 had improved cardiac function (FIG. 28B), exercise endurance (FIG. 28C), muscle force output (FIG. 28D), and reduced cardiac and skeletal muscle fibrosis (FIGS. 28E, 28F, respectively) compared to vehicle control.

FIGS. 29A-29D are a collection of schematic diagrams and graphs showing that TY4 improves outcomes in a mouse model of bleomycin-induced scleroderma. FIG. 29A: mouse model of bleomycin-induced scleroderma. Animals receiving intravenous TY4 post bleomycin-induced tissue damage had improved exercise endurance (FIG. 29B), lower pulmonary edema (as demonstrated by reduced lung weight to body weight ratio; FIG. 29C) and lower fibrosis in tissue (FIG. 29D).

FIG. 30 is a schematic diagram showing a predicted mechanism for TY4-induced suppression of both hypertrophic and pro-fibrotic cascades.

FIGS. 31A-31D are a collection of graphs showing that TY4 inhibits inflammatory activation in macrophages. Pro-inflammatory gene expression was measured for Nos2 (FIG. 31A), IL6 (FIG. 31B), IL-1b (FIG. 31C), and IL12b (FIG. 31D).

FIG. 32 is a collection of graphs showing that TY4, but not a scrambled version, reverses diastolic dysfunction in HFpEF mice. ***p:<0.001.

FIGS. 33A-33D are a collection of graphs showing that intravenous TY4 reverses disease manifestations in a mouse model of HCM. Four-month-old cTNI^{146Gly} mice were given intravenous biweekly infusions of TY4 for four weeks and (FIG. 33A) had dramatically reduced intraventricular septum thickness (FIG. 33B), attenuated blood velocity (as measured by pulse wave doppler; FIG. 33C), improved exercise endurance (FIG. 33D), and reduced circulating inflammatory markers (BNP; FIG. 33E).

FIG. 34 is a schematic diagram showing formulations for IV and oral administration of TY4.

FIGS. 35A-35E are a collection of graphs and images showing cardioprotective activity of TY4 by IV and oral administration in a model of myocardial infarction. FIGS. 35A and 35D are graphs showing infarct size. FIG. 35B is an imaging showing representative TTC-stained sections for each group 48 hours post-MI. FIGS. 35C and 35E are graphs showing circulating levels of cardiac troponin levels in animals 48 hours post injury.

FIGS. 36A-36E are a collection of graphs showing efficacy of orally administered TY4 in a model of scleroderma. FIG. 36A is a graph comparing exercise endurance in healthy animals (“control”), oral vehicle-treated animals with scleroderma (“PBS”), oral scramble-treated animals with scleroderma, and oral TY4-treated animals with scleroderma. FIG. 36B is a graph comparing body weight at end point in the different treatment groups. FIG. 36C is a graph comparing relative heart weight at end point in the different treatment groups. FIG. 36D is a graph comparing relative lung weight at end point in the different treatment groups. FIG. 36E is a graph comparing lung weight at end point in the different treatment groups.

FIGS. 37A-37D are a collection of images and graphs showing efficacy of orally administered TY4 in a model of scleroderma. FIG. 37A is a collection of images showing representative stained heart tissue sections in healthy animals (“control”), oral vehicle-treated animals with scleroderma (“PBS”), oral scramble-treated animals with scleroderma, and oral TY4-treated animals with scleroderma. FIG. 37B is a graph comparing the extent of heart fibrosis at end point in the different treatment groups. FIG. 37C is a collection of images showing representative stained skin tissue sections in the different treatment groups. FIG. 37D is a graph comparing dermal thickness in the different treatment groups.

FIG. 38 is a collection of graphs showing efficacy of orally administered TY4 in a model of scleroderma.

FIGS. 39A and 39B are a collection of a graph and images showing efficacy of orally administered TY4 in a model of HFpEF.

FIGS. 40A-40H are a collection of graphs and images showing the therapeutic benefits of orally-delivered TY4. FIG. 40A is a graph comparing left ventricular ejection fraction (EF) measured by transthoracic echocardiography in different treatment groups. FIG. 40B is a collection of images and a graph comparing myocardial fibrosis in different treatment groups using Masson's trichrome staining. FIGS. 40C and 40H are graphs comparing torque output of the anterior crural muscles in different treatment groups. FIG. 40D is a collection of images and a graph comparing skeletal muscle fibrosis in different treatment groups using Masson's trichrome staining. FIG. 40E is a graph comparing the number of myofibers in different treatment groups. FIG. 40F and FIG. 40G shows increased in exercise endurance and left ventricular ejection fraction within each group at eight weeks post treatment (respectively).

FIGS. 41A-41D are a collection of schematic diagrams and graphs showing in vitro and in vivo potency assays for TY4. FIG. 41A is a schematic diagram outlining in vitro and in vivo potency assays for TY4 using mouse bone marrow derived macrophages (BMDMs). FIG. 41B is a graph comparing p21 expression in LPS-activated BMDMs. FIG. 41C is a graph comparing NFkB expression in LPS-activated BMDMs. FIG. 41D is a graph comparing IL-6 expression in LPS-activated BMDMs.

FIG. 42 is a schematic diagram outlining optimizing the dosing strategy for oral delivery of TY4 in a model of HCM.

FIGS. 43A-43C are a collection of schematic diagrams and a graph showing assessing the biodistribution and pharmacokinetics of oral-TY4. FIG. 43A is a schematic diagram showing a study design for assessing the biodistribution of oral-TY4.

FIG. 43B is a schematic diagram showing a study design for assessing the biodistribution of oral-TY4. FIG. 43C is a graph showing abundance of retro-orbitally administered TY4 in different tissues by qPCR.

FIG. 44 is a schematic diagram outlining evaluation of the safety and toxicology profile of oral TY4.

FIGS. 45A-45E are a collection of graphs showing that TY4 binds Translocated Protein Region (TPR, a nuclear pore complex protein) and mediates its autophagy. FIG. 45A is a graph comparing sub-cellular localization of TY4 in bone marrow-derived macrophages upon activation with LPS. FIG. 45B is a graph showing proteins that bind to TY4 in HUVECs. FIG. 45C is a graph showing pull down of TPR by TY4 in HUVECs. FIG. 45D is a collection of graphs showing sequencing of TPR transcripts. FIG. 45E is a collection of graphs showing proteins that interact with TPR in TY4-exposed macrophages.

FIGS. 46A and 46B are a schematic diagram and a graph, respectively, collectively showing that TPR knockdown alone is cardioprotective as shown in a rat model of myocardial infarction.

### DETAILED DESCRIPTION

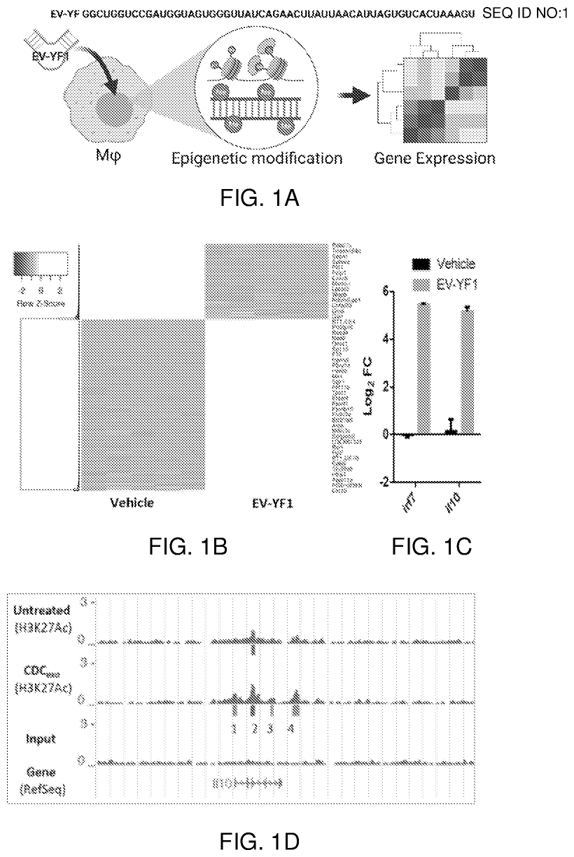

Non-coding RNA (ncRNA) in CDC-EVs are implicated in disease-modifying bioactivity of CDC-EV. Among ncRNA, Y RNAs are of interest as they are abundant in CDC-EVs (18% of small RNAs). EV-YF1 is a ncRNA found in CDC-EV and encoded by the human Y-RNA4 gene. EV-YF1 increases secretion of interleukin 10 (IL-10), an anti-inflammatory cytokine, by macrophages and is cardioprotective against myocardial infarction (MI). EV-YF1 is also antifibrotic and anti-hypertrophic in a model of hypertension and hypertrophy induced by angiotensin II infusion. The nucleotide sequence of EV-YF1 is provided in SEQ ID NO: 1 (see FIG. 1A). The present disclosure provides nucleic acids, e.g., TY4, that are bioinspired by EV-YF1, for example, to improve stability and potency as a therapeutic agent. TY4 (SEQ ID NO:4) is a chemically modified variant of EV-YF1 and has strong disease-modifying bioactivity in a number of diseases associated with inflammation and fibrosis, such as heart failure, hypertrophic cardiomyopathy, heart failure with preserved ejection fraction (HFpEF), Duchenne muscular dystrophy, or scleroderma. Without being bound by theory, TY4 is thought to specifically bind to translocated promoter region (TPR), a nuclear pore complex protein, to bring about its disease-modifying activity.

Provided herein is an isolated nucleic acid, e.g., RNA, that includes a nucleotide sequence of CGUCCGAUGGUAGUGGGUUAUCAG (SEQ ID NO: 12), sequence variants and/or chemical modifications thereof, and methods of use thereof. As use herein, “chemical modification” refers to a chemical difference in the structure of the nucleotides of the nucleic acid relative to the corresponding basic nucleotides (e.g., adenine, guanine, uracil, thymidine, cytosine). A chemical modification can be a natural modification or an artificial modification of the chemical structure of the basic nucleotides. In some embodiments, the isolated nucleic acid includes at least one non-natural chemical modification. In general, the present nucleic acids are 30 nt long or shorter (e.g., 16-30 nt long, or 24-30 nt long). In some embodiments, the nucleic acid is TY4 (SEQ ID NO: 2), or a sequence variant thereof. In general, a variant, e.g., a sequence variant or chemical modification, of the nucleic acid includes variants and chemical modifications that are functional. Thus, sequence variations and chemical modifications contemplated are those that substantially preserve the therapeutic potency of the original molecule (e.g., having a nucleotide sequence of SEQ ID NO: 12 or SEQ ID NO: 2). In some embodiments, an isolated nucleic acid of the present disclosure is

Nucleic acids of the present disclosure, e.g., TY4 and/or variants thereof, find use in treating conditions where inflammation and/or tissue injury are the main drivers of pathology. In some embodiments, the nucleic acids of the present disclosure, e.g., TY4 and/or variants thereof, treat diseases and conditions that are characterized by inflammation and/or fibrosis. “Fibrosis” as used herein can include any remodeling (e.g., pathological remodeling) of tissue (e.g., connective tissue, skeletal muscle, myocardium, skin), such as, but not limited to, deposition of fibrotic and/or fatty tissue, replacement of muscle tissue with fibrotic and/or fatty tissue, etc. In some embodiments, conditions treated by nucleic acids of the present disclosure, e.g., TY4 and/or variants thereof, include, without limitation, inflammatory disease, muscular dystrophy, or cardiac injury. In some embodiments, the present nucleic acids, e.g., TY4 and/or variants thereof, have cardioprotective effects when administered to a subject suffering from cardiac injury due to, without limitation, myocardial infarction and/or heart failure. Without being bound by theory, the nucleic acids, such as TY4, can increase an anti-inflammatory activity of macrophages, e.g., by promoting secretion of interleukin 10 (IL-10) from macrophages. In some embodiments, nucleic acids of the present disclosure induce changes in expression of one or more gene products and/or epigenetic changes in macrophages that are exposed to the nucleic acids. In some embodiments, conditions, e.g., inflammatory disease, muscular dystrophy, or cardiac injury, treated by the nucleic acids of the present disclosure, e.g., TY4 and/or variants thereof, are conditions that are responsive to anti-inflammatory effects of IL-10. Without being bound by theory, the nucleic acids, such as TY4, can suppress hypertrophic and/or pro-fibrotic signaling cascades, e.g., in injured tissue.

Without being bound by theory and as noted above, it is thought that TY4 exhibits its therapeutic effect by specifically binding translocated promoter region (TPR). Thus, wherever TY4 is discussed in the present disclosure, any agent, e.g., nucleic acid, that specifically binds to TPR is also contemplated. In any of the treatment methods, in some embodiments, the method of treatment contemplates administering TPR-binding nucleic acid, e.g., TY4, such as an inhibitory nucleic acid (e.g., siRNA) against TPR to a subject in need thereof.

In some embodiments, nucleic acids of the present disclosure are chemically modified to increase stability, e.g., in vivo and/or in vitro stability. In some embodiments, nucleic acids of the present disclosure are chemically modified to reduce immunogenicity. In some embodiments, the chemical modification of the nucleic acid increases the therapeutic activity of the nucleic acid. In some embodiments, nucleic acids of the present disclosure have enhanced therapeutic potency compared to endogenously encoded RNA molecules, e.g., endogenously encoded Y RNA fragments such as EV-YF1. Nucleic acids of the present disclosure in some embodiments can be provided in a composition (e.g., pharmaceutical compositions) or a kit.

The therapeutic nucleic acids of the present disclosure can be administered by any suitable route, including, without limitation, intravenously or orally. Provided herein are intravenous or oral formulations for administration of nucleic acids of the present disclosure, e.g., TY4, and the use of same for treatment of a condition associated with inflammation and/or fibrosis.

### Definitions

As used herein the term “nucleic acid” or “oligonucleotide” refers to multiple nucleotides (e.g., molecules comprising a sugar (e.g. ribose or deoxyribose) linked to a phosphate group and to an exchangeable organic base, which is either a substituted pyrimidine (e.g. cytosine (C), thymidine (T) or uracil (U)) or a substituted purine (e.g. adenine (A) or guanine (G)). The term includes polynucleosides (i.e. a polynucleotide minus the phosphate) and any other organic base containing polymer. Purines and pyrimidines include but are not limited to adenine, cytosine, guanine, thymidine, inosine, 5-methylcytosine, 2-aminopurine, 2-amino-6-chloropurine, 2,6-diaminopurine, hypoxanthine, and other naturally and non-naturally occurring nucleobases, substituted and unsubstituted aromatic moieties. A nucleic acid can include any other suitable modifications. Thus, the term nucleic acid also encompasses nucleic acids with substitutions or modifications, such as in the bases and/or sugars.

Polypeptide or nucleic acid molecules of the present disclosure may share a certain degree of sequence similarity or identity with the reference molecules (e.g., reference polypeptides or reference polynucleotides), for example, with art-described molecules (e.g., engineered or designed molecules or wild-type molecules). The term “identity” as known in the art, refers to a relationship between the sequences of two or more polypeptides or polynucleotides, as determined by comparing the sequences. In the art, identity also means the degree of sequence relatedness between them as determined by the number of matches between strings of two or more amino acid residues or nucleic acid residues. Identity measures the percent of identical matches between the smaller of two or more sequences with gap alignments (if any) addressed by a particular mathematical model or computer program (e.g., “algorithms”). Identity of related peptides can be readily calculated by known methods. “% identity” as it applies to polypeptide or polynucleotide sequences is defined as the percentage of residues (amino acid residues or nucleic acid residues) in the candidate amino acid or nucleic acid sequence that are identical with the residues in the amino acid sequence or nucleic acid sequence of a second sequence after aligning the sequences and introducing gaps, if necessary, to achieve the maximum percent identity. Any suitable methods and computer programs for the alignment can be used. It is understood that identity depends on a calculation of percent identity but may differ in value due to gaps and penalties introduced in the calculation. Generally, variants of a particular polynucleotide or polypeptide have at least 40%, 45%, 50%, 55%, 60%, 65%, 70%, 75%, 80%, 85%, 90%, 91%, 92%, 93%, 94%, 95%, 96%, 97%, 98%, 99% but less than 100% sequence identity to that particular reference polynucleotide or polypeptide as determined by sequence alignment programs and parameters described herein and known to those skilled in the art. Such tools for alignment include those of the BLAST suite (Stephen F. Altschul, et al (1997), “Gapped BLAST and PSI-BLAST: a new generation of protein database search programs”. Nucleic Acids Res. 25:3389-3402). Another popular local alignment technique is based on the Smith-Waterman algorithm (Smith, T. F. & Waterman, M. S. (1981) “Identification of common molecular subsequences.” J. Mol. Biol. 147:195-197.) A general global alignment technique based on dynamic programming is the Needleman-Wunsch algorithm (Needleman, S. B. & Wunsch. C. D. (1970) “A general method applicable to the search for similarities in the amino acid sequences of two proteins.” J. Mol. Biol. 48:443-453.). More recently a Fast Optimal Global Sequence Alignment Algorithm (FOGSAA) has been developed that purportedly produces global alignment of nucleotide and protein sequences faster than other optimal global alignment methods, including the Needleman-Wunsch algorithm. Other tools are described herein, specifically in the definition of “identity” below.

The term “identity” refers to the overall relatedness between polymeric molecules, for example, between polynucleotide molecules (e.g. DNA molecules and/or RNA molecules) and/or between polypeptide molecules. Calculation of the percent identity of two polynucleic acid sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a suitable mathematical algorithm. For example, the percent identity between two nucleic acid sequences can be determined using methods such as those described in Computational Molecular Biology, Lesk, A. M., ed., Oxford University Press, New York, 1988; Biocomputing: Informatics and Genome Projects. Smith. D. W., ed., Academic Press. New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleic acid sequences can be determined using the algorithm of Meyers and Miller (CABIOS, 1989, 4:11-17), which has been incorporated into the ALIGN program (version 2.0) using a PAM 120 weight residue table, a gap length penalty of 12 and a gap penalty of 4. The percent identity between two nucleic acid sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)).

The term “Watson-Crick base-pairing”, or “base-pairing” refers to the formation of hydrogen bonds between specific pairs of nucleotide bases (“complementary base pairs”). For example, two hydrogen bonds form between adenine (A) and uracil (U), and three hydrogen bonds form between guanine (G) and cytosine (C). One method of assessing the strength of bonding between two polynucleotides is by quantifying the percentage of bonds formed between the guanine and cytosine bases of the two polynucleotides (“GC content”). In some embodiments, the GC content of bonding between two nucleic acids of a multimeric molecule (e.g., a multimeric mRNA molecule) is at least 10%, at least 20%, at least 30%, at least 40%, or at least 50%. In some embodiments, the GC content of bonding between two nucleic acids of a multimeric molecule (e.g., a multimeric mRNA molecule) is between 10% and 70%, about 20% to about 60%, or about 30% to about 60%. The formation of a nucleic acid duplex via bonding of complementary base pairs can also be referred to as “hybridization”. Generally, two nucleic acids sharing a region of complementarity are capable, under suitable conditions, of hybridizing (e.g., via nucleic acid base pairing) to form a duplex structure. A region of complementarity can vary in size. In some embodiments, a region of complementarity ranges in length from about 2 base pairs to about 100 base pairs. In some embodiments, a region of complementarity ranges in length from about 5 base pairs to about 75 base pairs. In some embodiments, a region of complementarity ranges in length from about 10 base pairs to about 50 base pairs. In some embodiments, a region of complementarity ranges in length from about 20 base pairs to about 30 base pairs.

“Isolated” as used herein with reference to an isolated biomolecule, e.g., a nucleic acid, has the ordinary and customary meaning to one of ordinary skill in the art in view of the present disclosure. An isolated biomolecule, e.g., an isolated nucleic acid, is generally in a non-natural environment, or in an environment that the biomolecule would otherwise not have been without human intervention of the biomolecule or its environment. In some embodiments, an isolated biomolecule is not inside a cell or an organism.

“Extracellular vesicle” or “EV” as used herein have their ordinary and customary meaning as understood by one of ordinary skill in the art, in view of the present disclosure. EVs include lipid bilayer structures generated by cells, and include exosomes, microvesicles, epididimosomes, argosomes, exosome-like vesicles, microparticles, promininosomes, prostasomes, dexosomes, texosomes, dex, tex, archeosomes and oncosomes.

“Micelle,” as used herein with reference to casein micelles, has its customary and ordinary meaning as understood by one of ordinary skill in the art, in view of the present disclosure. Casein micelles are colloidal particles that can include aggregates of one or more casein phosphoproteins (e.g., one or more, two or more, three or more, or all four of alpha s1 casein, alpha s2 casein, beta casein, and kappa casein).

“Subject,” as used herein refers to any vertebrate animal, including mammals and non-mammals. A subject can include primates, including humans, and non-primate mammals, such as rodents, domestic animals or game animals. Non-primate mammals can include mouse, rat, hamster, rabbit, dog, fox, wolf, cat, horse, cow, pig, sheep, goat, camel, deer, buffalo, bison, etc. Non-mammals can include bird (e.g., chicken, ostrich, emu, pigeon), reptile (e.g., snake, lizard, turtle), amphibian (e.g., frog, salamander), fish (e.g., salmon, cod, pufferfish, tuna), etc. The terms, “individual,” “patient,” and “subject” are used interchangeably herein.

“Administering” as used herein can include any suitable routes of administering a therapeutic agent or composition as disclosed herein. Suitable routes of administration include, without limitation, oral, parenteral, intravenous, intramuscular, subcutaneous, transdermal, airway (aerosol), pulmonary, cutaneous, injection or topical administration. Administration can be local or systemic.

As used herein, “treat” and “treatment” includes curing, improving, ameliorating, reducing the severity of, preventing, slowing the progression of, and/or delaying the appearance of a disease, condition and/or symptoms thereof.

A treatment can be considered “effective,” or “therapeutically effective” as used herein, if one or more of the signs or symptoms of a condition described herein are altered in a beneficial manner, other clinically accepted symptoms are improved, or even ameliorated, or a desired response is induced e.g., by at least 2%, 3%, 4%, 5%, 10%, or more, following treatment according to the methods described herein. Efficacy can be assessed, for example, by measuring a marker, indicator, symptom, and/or the incidence of a condition treated according to the methods described herein or any other measurable parameter appropriate, e.g. exercise endurance. Efficacy can also be measured by a failure of an individual to worsen as assessed by hospitalization, or need for medical interventions (e.g., progression of the disease is halted). Treatment includes any treatment of a disease or condition in an individual or an animal (some non-limiting examples include a human or an animal) and includes: (1) inhibiting the disease or condition, e.g., preventing a worsening of symptoms (e.g. pain or inflammation); or (2) relieving the severity of the disease or condition, e.g., causing regression of symptoms. An effective amount for the treatment of a disease or condition means that amount which, when administered to a subject in need thereof, is sufficient to result in effective treatment as that term is defined herein, for that disease or condition. Efficacy of an agent can be determined by assessing physical indicators of a condition or desired response, (e.g. muscle function, mass or volume). One skilled in the art can monitor efficacy of administration and/or treatment by measuring any one of such parameters, or any combination of parameters.

The term “effective amount” or “therapeutically effective amount” as used herein refers to the amount of a composition or an agent needed to alleviate at least one or more symptom of the disease or condition, and relates to a sufficient amount of therapeutic composition to provide the desired effect. The term “effective amount” or “therapeutically effective amount” can refer to an amount of a composition or therapeutic agent that is sufficient to provide a particular anti-inflammatory and/or cardioprotective effect when administered to a typical subject. An effective amount as used herein, in various contexts, can include an amount sufficient to delay the development of a symptom of the disease or condition, alter the course of a symptom disease or condition (for example but not limited to, slowing the progression of a symptom of the disease or condition), or reverse a symptom of the disease or condition. In some embodiments, the therapeutically effective amount is administered in one or more doses of the therapeutic agent. In some embodiments, the therapeutically effective amount is administered in a single administration, or over a period of time in a plurality of doses.

As used herein, the phrase “physiologically compatible” and “pharmaceutically acceptable” are employed interchangeably herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Definitions of common terms in cell biology and molecular biology can be found in “The Merck Manual of Diagnosis and Therapy”, 19th Edition, published by Merck Research Laboratories, 2006 (ISBN 0-91 1910-19-0); Robert S. Porter et al. (eds.), The Encyclopedia of Molecular Biology, published by Blackwell Science Ltd., 1994 (ISBN 0-632-02182-9); Benjamin Lewin, Genes X, published by Jones & Bartlett Publishing, 2009 (ISBN-10: 0763766321); Kendrew et al. (eds.), Molecular Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds.

The singular terms “a,” “an,” and “the” include plural referents unless context clearly indicates otherwise. Similarly, the word “or” is intended to include “and” unless the context clearly indicates otherwise. The abbreviation, “e.g.” is used herein to indicate a non-limiting example. Thus, the abbreviation “e.g.” is synonymous with the term “for example.” The term “about” as used herein to, for example, define the values and ranges of molecular weights means that the indicated values and/or range limits can vary within ±20%, e.g., within ±10%, including within ±5%. The use of “about” before a number includes the number itself. For example, “about 5” provides express support for “5.” Numbers provided in ranges include overlapping ranges and integers in between; for example a range of 1-4 and 5-7 includes for example, 1-7, 1-6, 1-5, 2-5, 2-7, 4-7, 1, 2, 3, 4, 5, 6 and 7.

### Nucleic Acids

Provided herein is an isolated nucleic acid that includes a nucleotide sequence of CGUCCGAUGGUAGUGGGUUAUCAG (SEQ ID NO: 12), or a variant thereof. In some embodiments, the nucleic acid is RNA. In some embodiments, the nucleic acid includes a nucleotide sequence at least 80%, 85%, 90%, 92%, 93%, 94%, 95%, 96%, 98%, 99% identical to CGUCCGAUGGUAGUGGGUUAUCAG (SEQ ID NO: 12). In some embodiments, the nucleic acid includes a nucleotide sequence of CGUCCGAUGGUAGUGGGUUAUCAG (SEQ ID NO: 12) with a sequence variation at up to 1, 2, 3, 4, or 5 positions in the nucleotide sequence. As used herein, a “position” within a nucleotide sequence or nucleic acid is defined relative to the 5′ end of the nucleotide sequence or nucleic acid. In some embodiments, the nucleotide sequence of the nucleic acid is CGUCCGAUGGUAGUGGGUUAUCAG (SEQ ID NO: 12), or a sequence variant thereof. The nucleic acid can be any suitable length. In some embodiments, the nucleic acid is 24 nucleotides (nt) long. In some embodiments, the nucleic acid is 16, 17, 18, 19, 20, 21, 22, 23, 24, 25, 26, 27, 28, 29, 30, 31, 32, 33, 34, 35, 36, 37, 38, 39, 40 nt long, or longer. In some embodiments, the nucleic acid is at most 30 nt long. In some embodiments, the nucleic acid is 16-30 nt long, or 24-30 nt long.

A nucleic acid of the present disclosure can be single stranded or double stranded (e.g., RNA/DNA hybrid). In some embodiments, the nucleic acid is single stranded.

An isolated nucleic acid of the present disclosure in some embodiments includes one or more chemically-modified nucleotides, e.g., nucleotides with a modified backbone. In general, the chemical modification(s) is one that substantially preserves or enhances the therapeutic potency of the nucleic acid. Any suitable number of nucleotides of the nucleic acid can be chemically modified. In some embodiments, the nucleic acid includes 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more chemically-modified nucleotides. In some embodiments, the nucleic acid includes 1-5, 1-6, 1-7, 1-8, 1-9, 1-10, 1-15, 1-20, 1-25, or 1-30 chemically-modified nucleotides. In some embodiments, the nucleic acid includes 1-10 chemically-modified nucleotides. In some embodiments, the nucleic acid includes 8 chemically-modified nucleotides. In some embodiments, the nucleic acid includes 6 chemically-modified nucleotides.

The chemically modified nucleotides can be distributed along the isolated nucleic acid in any suitable manner. In some embodiments, the nucleic acid includes at least one chemically-modified nucleotide within the first half of the nucleic acid, e.g., the 5′ half of the nucleic acid. In some embodiments, the nucleic acid includes at least one chemically-modified nucleotide within the second half of the nucleic acid, e.g., the 3′ half of the nucleic acid. In some embodiments, the nucleic acid includes at least one chemically-modified nucleotide within the first half of the nucleic acid, e.g., the 5′ half of the nucleic acid, and at least one chemically-modified nucleotide within the second half of the nucleic acid, e.g., the 3′ half of the nucleic acid. In some embodiments, the nucleic acid includes one or more chemically-modified nucleotides within 1, 2, 3, 4, 5, 6, 7, 8, 9, 10 or more nucleotides from the 5′ end of the nucleic acid. In some embodiments, the nucleic acid includes one or more chemically-modified nucleotides within 1, 2, 3, 4, 5, 6, 7, 8, 9, 10 or more nucleotides from the 3′ end of the nucleic acid. In some embodiments, no two chemically-modified nucleotides are adjacent each other in the nucleic acid. In some embodiments, the nucleic acid includes 1, 1, 2, 2, 3, 3, 4, 4, 5, 5 chemically-modified nucleotides within 1, 2, 3, 4, 5, 6, 7, 8, 9, 10 nucleotides, respectively, from the 5′ end of the nucleic acid. In some embodiments, the nucleic acid includes 1, 1, 2, 2, 3, 3, 4, 4, 5, 5 chemically-modified nucleotides within 1, 2, 3, 4, 5, 6, 7, 8, 9, 10 nucleotides, respectively, from the 3′ end of the nucleic acid. In some embodiments, the nucleic acid includes the same number of chemically-modified nucleotides in the 5′ half and 3′ half of the nucleic acid. In some embodiments, the nucleic acid includes 3 chemically-modified nucleotides within 5 nucleotides from the 5′ end of the nucleic acid and/or 3 chemically-modified nucleotides within 5 nucleotides from the 3′ end of the nucleic acid.

In some embodiments, the chemically-modified nucleotides are within the nucleotide sequence of CGUCCGAUGGUAGUGGGUUAUCAG (SEQ ID NO: 12), or sequence variant thereof. In some embodiments, the nucleic acid includes at least one chemically-modified nucleotide within the first half of the nucleotide sequence, e.g., the 5′ half of the nucleotide sequence. In some embodiments, the nucleic acid includes at least one chemically-modified nucleotide within positions 1-12 of the nucleotide sequence. In some embodiments, the nucleic acid includes at least one chemically-modified nucleotide within the second half of the nucleotide sequence, e.g., the 3′ half of the nucleotide sequence. In some embodiments, the nucleic acid includes at least one chemically-modified nucleotide within positions 13-24 of the nucleotide sequence. In some embodiments, the nucleic acid includes at least one chemically-modified nucleotide within positions 1-12 of the nucleotide sequence, and at least one chemically-modified nucleotide within positions 13-24 of the nucleotide sequence. In some embodiments, the nucleic acid includes one or more chemically-modified nucleotides within 1, 2, 3, 4, 5, 6, 7, 8, 9, 10 or more nucleotides from the 5′ end of the nucleotide sequence. In some embodiments, the nucleic acid includes one or more chemically-modified nucleotides within 1, 2, 3, 4, 5, 6, 7, 8, 9, 10 or more nucleotides from the 3′ end of the nucleotide sequence. In some embodiments, no two chemically-modified nucleotides are adjacent each other in the nucleotide sequence. In some embodiments, the nucleic acid includes 1, 1, 2, 2, 3, 3, 4, 4, 5, 5 chemically-modified nucleotides within 1, 2, 3, 4, 5, 6, 7, 8, 9, 10 nucleotides, respectively, from the 5′ end of the nucleotide sequence. In some embodiments, the nucleic acid includes 1, 1, 2, 2, 3, 3, 4, 4, 5, 5 chemically-modified nucleotides within 1, 2, 3, 4, 5, 6, 7, 8, 9, 10 nucleotides, respectively, from the 3′ end of the nucleotide sequence. In some embodiments, the nucleic acid includes the same number of chemically-modified nucleotides in the 5′ half and 3′ half of the nucleotide sequence. In some embodiments, the nucleic acid includes 3 chemically-modified nucleotides within 5 nucleotides from the 5′ end of the nucleotide sequence and/or 3 chemically-modified nucleotides within 5 nucleotides from the 3′ end of the nucleotide sequence. In some embodiments, the nucleic acid includes a different number of chemically-modified nucleotides in the 5′ half and 3′ half of the nucleotide sequence. In some embodiments, the nucleic acid includes a greater number of chemically-modified nucleotides in the 3′ half than in the 5′ half of the nucleotide sequence. In some embodiments, the nucleic acid includes 3 chemically-modified nucleotides within 5 nucleotides from the 5′ end of the nucleotide sequence and/or 3, 4, or 5 chemically-modified nucleotides within 5 nucleotides from the 3′ end of the nucleotide sequence.

In some embodiments, the isolated nucleic acid includes a chemically-modified nucleotide at one or more of positions 1, 3, 5, 20, 22 and 24 of the nucleotide sequence. In some embodiments, the isolated nucleic acid includes a chemically-modified nucleotide at positions 1, 3, 5, 20, 22 and 24 of the nucleotide sequence. In some embodiments, the isolated nucleic acid has the nucleotide sequence CGUCCGAUGGUAGUGGGUUAUCAG (SEQ ID NO: 12), or a sequence variant thereof, where one or more of positions 1, 3, 5, 20, 22, and 24 are chemically modified. In some embodiments, the chemically-modified nucleotide(s) increases in vitro and/or in vivo stability of the nucleic acid. In some embodiments, the chemically-modified nucleotide(s) increases therapeutic potency of the nucleic acid, e.g., for treating an inflammatory condition, cardiac injury, or muscular dystrophy.

The isolated nucleic acid in some embodiments includes one type, or two or more different types of chemically-modified nucleotides. In some embodiments, the chemically-modified nucleotide has a methylene bridge connecting the 2′-O atom and the 4′-C atom of the nucleotide sugar ring to lock the conformation (Locked Nucleic Acid (LNA)). In some embodiments, the isolated nucleic acid includes the nucleotide sequence CGUCCGAUGGUAGUGGGUUAUCAG (SEQ ID NO: 12), or a sequence variant thereof, where one or more of positions 1, 3, 5, 20, 22, and 24 are LNA. In some embodiments, the isolated nucleic acid has the nucleotide sequence CGUCCGAUGGUAGUGGGUUAUCAG (SEQ ID NO: 12), or a sequence variant thereof, where one or more of positions 1, 3, 5, 20, 22, and 24 are LNA. In some embodiments, the isolated nucleic acid includes the nucleotide sequence CGUCCGAUGGUAGUGGGUUAUCAG (SEQ ID NO: 2), or a sequence variant thereof, where positions 1, 3, 5, 20, 22, and 24 are LNA. In some embodiments, the isolated nucleic acid has the nucleotide sequence CGUCCGAUGGUAGUGGGUUAUCAG (SEQ ID NO: 2), where positions 1, 3, 5, 20, 22, and 24 are LNA.

The isolated nucleic acid, in some embodiments, can include any suitable chemical modification. In some embodiments, the chemical modification is a backbone modification, e.g., modification of the sugar/phosphate backbone. In some embodiments, the chemical modification is a backbone sugar modification. In some embodiments, the chemically modified nucleotide includes a LNA. In some embodiments, the chemical modification includes the introduction of a phosphorothioate group as linker between nucleotides. Suitable backbone modifications of the chemically-modified nucleotides include, without limitation, phosphorothioates, phosphotriesters, methyl phosphonates, short chain alkyl or cycloalkyl intersugar linkages or short chain heteroatomic or heterocyclic intersugar linkages. In some embodiments, the chemical modification is a base modification.

The nucleic acids of the present disclosure can be prepared using any suitable option. Suitable options include, without limitation, chemical synthesis, enzymatic production and/or biological production. In some embodiments, the nucleic acids are prepare using chemical synthesis. Any suitable option for chemical synthesis of nucleic acids can be used. Suitable options include, without limitation, phosphodiester, phosphotriester, phosphoramidite, phosphite-triester, and solid phase synthesis approaches. In some embodiments, preparing the nucleic acids includes in vitro transcription. In some embodiments, the nucleic acids are prepared using recombinant DNA technology. In some embodiments, the nucleic acids are prepared by chemically modifying an unmodified nucleic acid having a nucleotide sequence of interest.

### Compositions

Also provided herein are compositions that include the nucleic acid of the present disclosure. In some embodiments, the composition is a pharmaceutical composition. In some embodiments the composition includes pharmaceutically acceptable excipient. In some embodiments, the composition is a cell-free composition, e.g., the composition is substantially free of cells such as CDC. In some embodiments, the composition is an extracellular vesicle-free composition, e.g., the composition is substantially free of extracellular vesicles, such as exosomes.

Some non-limiting examples of materials which can serve as pharmaceutically-acceptable excipients include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations.

In some embodiments, the composition includes a transfection reagent, e.g., to promote delivery of the nucleic acid to a target cellular target (in vitro or in vivo). Any suitable transfection reagent can be included in the composition. Suitable transfection reagents include, without limitation, a liposome, extracellular vesicle (EV), and a polyethylene glycol (PEG)-cationic lipid complex (PCLC). In some embodiments, the transfection reagent includes a lipid (e.g., a liposome-forming lipid), or a PEGylated lipid. In some embodiments, the lipid is a cationic lipid, as provided herein. In some embodiments, the transfection reagent includes DharmaFECT® or Lipofectamine®. In some embodiments, the nucleic acid of the present disclosure is formulated with the transfection reagent in the composition so as to promote cellular uptake and/or pharmacokinetics of the nucleic acid.

Liposomes are artificially-prepared vesicles which may primarily be composed of a lipid bilayer and may be used as a delivery vehicle for the administration of pharmaceutical formulations. Liposomes can be of different sizes such as, but not limited to, a multilamellar vesicle (MLV), which may be hundreds of nanometers in diameter and may contain a series of concentric bilayers separated by narrow aqueous compartments, a small unicellular vesicle (SUV), which may be smaller than 50 nm in diameter, and a large unilamellar vesicle (LUV), which may be between 50 and 500 nm in diameter. Liposome design may include, without limitation, opsonins or ligands in order to improve the attachment of liposomes to target tissue/cells, or to activate events such as, but not limited to, endocytosis. Liposomes may contain a low or a high pH in order to improve the delivery of the cargo, e.g., a nucleic acid of the present disclosure.

In some embodiments, the composition includes, without limitation, liposomes such as those formed from 1,2-dioleyloxy-N,N-dimethylaminopropane (DODMA) liposomes, DiLa2 liposomes from Marina Biotech (Bothell, Wash.), 1,2-dilinoleyloxy-3-dimethylaminopropane (DLin-DMA), 2,2-dilinoleyl-4-(2-dimethylaminoethyl)-[1,3]-dioxolane (DLin-KC2-DMA), and MC3 and liposomes such as, but not limited to, DOXIL® from Janssen Biotech, Inc. (Horsham, Pa.).

In some embodiments, the composition includes a cationic lipid. Any suitable cationic lipid may be used in the present compositions. Suitable cationic lipids include, without limitation, DLin-DMA, DLin-D-DMA, DLin-MC3-DMA, DLin-KC2-DMA, DODMA and amino alcohol lipids. In some embodiments, the composition includes a cationic lipid complex, e.g., a polyethylene glycol (PEG)-cationic lipid complex (PCLC). In some embodiments, the cationic lipid is PEGylated, e.g., 2 kDa PEG (“PEG2000”). Any suitable option can be used to PEGylate the cationic lipid. In some embodiments, PCLC is formed by exposing a mixture of PEG and the cationic lipid to one or more freeze/thaw cycles, e.g., 1, 2, 3, 4, 5 or more freeze/thaw cycles. In some embodiments, a freeze/thaw cycle includes freezing the mixture with liquid nitrogen (e.g., around −190° C.) for about 5 minutes, and thawing at about 60° C. for about 5 minutes. A nucleic acid of the present disclosure can be mixed with the PCLC to generate a complex of the nucleic acid and the PCLC.

In some embodiments, the composition includes extracellular vesicles (EV), e.g., exosomes. The extracellular vesicles (EV) can be those from any suitable source, e.g., EV derived from cardiosphere-derived cells (CDC), or from fibroblasts. Suitable EV, such as CDC-derived EV, are provided in, e.g., U.S. Application Publication Nos. 20080267921, 20160158291 and 20160160181; Smith et al., Circulation. 2007. 115:896-908; Aminzadeh, M. A. et al. Stem Cell Reports 10, 942-955 (2018); and Ibrahim et al., Stem Cell Reports. 2014 May 8; 2(5):606-19, Ibrahim, A. G. et al. Nanomedicine 33, 102347 (2020), each of which is incorporated by reference in its entirety. In some embodiments, the EVs are those isolated from serum-free media conditioned by human CDCs in culture. In some embodiments, the composition includes EV and liposomes and/or PCLC as transfection reagents. In some embodiments, the composition is substantially free of CDC-derived EV.

EVs, e.g., exosomes, disclosed herein can vary in size, depending on the embodiment. Depending on the embodiment, the size of the EVs ranges in diameter from about 15 nm to about 95 nm in diameter, including about 15 nm to about 20 nm, about 20 nm to about 30 nm, about 30 nm to about 40 nm, about 40 nm to about 50 nm, about 50 nm to about 60 nm, about 60 nm to about 70 nm, about 70 nm to about 80 nm, about 80 nm to about 90 nm, about 90 nm to about 95 nm, and overlapping ranges thereof. In several embodiments, EVs are larger (e.g., those ranging from about 140 to about 210 nm, including about 140 nm to about 150 nm, about 150 nm to about 160 run, about 160 nm to about 170 nm, about 170 nm to about 180 nm, about 180 nm to about 190 nm, 190 nm to about 200 nm, about 200 nm to about 210 nm, and overlapping ranges thereof). In some embodiments, the EV diameter is in a range of about 15 nm to about 200 nm in diameter, including about 15 nm to about 20 nm, about 20 nm to about 30 nm, about 30 nm to about 40 nm, about 40 nm to about 50 nm, about 50 nm to about 60 nm, about 60 nm to about 70 nm, about 70 nm to about 80 nm, about 80 nm to about 90 nm, about 90 nm to about 100 nm, about 100 nm to about 110 nm, about 110 nm to about 120 nm, about 120 nm to about 130 nm, about 130 nm to about 140 nm, about 140 nm to about 150 nm, about 150 nm to about 160 nm, about 160 nm to about 170 nm, about 170 nm to about 180 nm, about 180 nm to about 190 nm, about 190 nm to about 200 nm, and overlapping ranges thereof. In some embodiments, the EVs that are generated from the original cellular body are 100, 200, 300, 400, 500, 600, 700, 800, 900, 1,000, 2,000, 5,000, or 10,000 times smaller in at least one dimension (e.g., diameter) than the original cellular body.

The composition containing the EV and nucleic acid of the present disclosure can be prepared using any suitable option. In some embodiments, loading the nucleic acid into the EV includes: formulating the nucleic acid with liposomes and/or PCLC, e.g., as provided above, to generate a nucleic acid-liposome mixture; combining the nucleic acid-liposome mixture with the EV; and enriching for EV associated with exosome markers to generate a population of EV enriched for the nucleic acid. Combining the nucleic acid-liposome mixture with the EV can be done using any suitable option. In some embodiments, the nucleic acid-liposome mixture is combined with the EV at 37° C. with shaking for about 30 minutes or more. Enriching to generate a population of EV enriched for the nucleic acid can be done using any suitable option. In some embodiments, enriching for EV associated with exosome markers includes immunoprecipitating EV associated with exosome markers using antibodies specific to an exosome marker. In some embodiments, the exosome marker is one or more of CD9, CD63 and CD81. In some embodiments, enriching for EV associated with exosome markers includes immunoprecipitating EV associated with all the exosome markers, CD9, CD63 and CD81. In some embodiments, the size distribution of the population of EV enriched for the nucleic acid is substantially unimodal. In some embodiments, at least 80%, 85%, 90%, 95%, 97%, 99% of the population has a diameter under a single peak in the size distribution. In some embodiments, the population of EV enriched for the nucleic acid has an average diameter of about 50-180 nm, e.g., 60-170 nm, 70-160 nm, 80-150 nm, 90-140 nm, 100-130 nm, or about 110-130 nm.

In some embodiments, the composition includes casein, e.g., a casein micelle. In some embodiments, the composition includes chitosan. In some embodiments, the composition includes casein and chitosan, e.g., a casein-chitosan micelle. In some embodiments, the composition includes a casein-chitosan complex. In some embodiments, the isolated nucleic acid in the composition is encapsulated in a casein-chitosan complex. In some embodiments, the composition includes one or more of phosphoproteins: alpha s1 casein, alpha s2 casein, beta casein, and kappa casein. In some embodiments, the composition includes two or more, three or more, or all four phosphoproteins: alpha s1 casein, alpha s2 casein, beta casein, and kappa casein. The phosphoproteins may be present in the composition at any suitable concentration (relative to each other, and relative to the total volume of the composition), and in some embodiments, is present in an amount suitable for forming casein micelles. In some embodiments, the casein phosphoproteins are collectively present in the composition at about 5-10% (weight by volume). In some embodiments, the casein phosphoproteins are collectively present in the composition at about 8% (weight by volume). In some embodiments, the casein phosphoproteins are collectively present in the composition at about 5% (weight by volume). The casein phosphoproteins can be those from any suitable animal, e.g., mammal such as, but not limited to, human, non-human primate, cow, pig, horse, camel, goat, and sheep. In some embodiments, the casein phosphoproteins are bovine alpha s1 casein, alpha s2 casein, beta casein, and kappa casein. Suitable casein formulations with EV are provided in, e.g., Aminzadeh et al., J Extracell Vesicles. 2021 January; 10(3):e12045, the entirety of which is incorporated herein by reference. In some embodiments, a composition, e.g., pharmaceutical composition, of the present disclosure formulated with casein, as provided herein, is suitable for oral administration to the subject. Without being bound by theory, the casein phosphoproteins in the composition are thought to increase the bioavailability of orally administered EV and/or liposomes and their cargo, e.g., the nucleic acid of the present disclosure.

In several embodiments, a composition for enhancing the oral bioavailability of a therapeutic nucleic acid of the present disclosure comprises at least two phosphoproteins selected from alpha s1 casein, alpha s2 casein, beta casein, and kappa casein, where the phosphoproteins are present in an amount between about 5% to about 10% (weight by volume) of the composition, in a physiologically compatible excipient. In several embodiments, the composition includes the alpha s1 casein in an amount between about 0% to about 50% (e.g., about 10% to about 45%, about 20% to about 40%, about 25% to about 40%, about including 30% to about 40%) (by weight), the alpha s2 casein in an amount between about 0% to about 20% (e.g., about 5% to about 15%, about 7% to about 12%, including about 8% to about 12%) (by weight), the beta casein in an amount between about 0% to about 50% (e.g., about 10% to about 45%, about 20% to about 40%, about 25% to about 40%, about including 30% to about 40%) (by weight), and the kappa casein in an amount between about 0% to about 20% (e.g., about 5% to about 18%, about 8% to about 18%, including about 10% to about 15%) (by weight) of the phosphoprotein mass in the composition. The present compositions can provide for enhanced oral bioavailability of therapeutic nucleic acids, such as TY4.

In several embodiments, the formulations provided for herein are in the form of lipid-bound vesicles, e.g., micelles or liposomes, and can therefore include any suitable number of particles. In some embodiments, the amount of micelles (e.g., casein-chitosan coated micelles) is in a range of about 10⁶ to about 10¹⁰ particles, e.g., about 2×10⁶ to about 10¹⁰ particles, about 5×10⁶ to about 10¹⁰ particles, about 10⁷ to about 5×10⁹ particles, about 2×10⁷ to about 5×10⁹ particles, about 5×10⁷ to about 5×10⁹ particles, including about 1×10⁸ to about 2×10⁹ particles. In some embodiments, the amount of micelles (e.g., casein-chitin coated micelles) in the population is about 10⁶, about 2×10⁶, about 5×10⁶, about 10⁷, about 2×10⁷, about 5×10⁷, about 10⁸, about 2×10⁸, about 5×10⁸, about 10⁹, about 2×10⁹, about 5×10⁹, or about 10¹⁰ particles, or an amount in between any two of the preceding values.

In several embodiments, the composition comprises casein-chitosan coated lipid micelles, where the casein phosphoproteins are present in the composition in suitable amounts (e.g., suitable total amount of phosphoprotein mass in the composition, suitable proportions of phosphoproteins relative to each other). In some embodiments, the composition includes two, three, or all four phosphoproteins selected from alpha s1 casein, alpha s2 casein, beta casein, and kappa casein. In some embodiments, the amount of a phosphoprotein in the composition depends on the amount of one or more other phosphoprotein present in the composition.

In some embodiments, alpha s1 casein is a phosphoprotein associated with the gene name CSN1S1. The alpha s1 casein can be a CSN1S1 phosphoprotein from any suitable mammal. In some embodiments, the alpha s1 casein is bovine (Gene ID: 282208), porcine (Gene ID: 445514), equine (Gene ID: 100033982), ovine (Gene ID: 443382), caprine (Gene ID: 100750242), cameline (Gene ID: 105090954), or human (Gene ID: 1446). In some embodiments, the alpha s1 casein is a non-human alpha s1 casein. In some embodiments, the alpha s1 casein is a polypeptide having an amino acid sequence at least 80%, at least 85%, at least 90%, at least 95%, at least 98%, or about 100% identical to the sequence set forth in SEQ ID NO: 17.

In some embodiments, the composition includes any suitable amount of alpha s1 casein. In some embodiments, the composition includes the alpha s1 casein in an amount, by weight, between about 0% to about 50%, e.g., between about 5% to about 50%, between about 10% to about 50%, between about 15% to about 45%, between about 20% to about 45%, including between about 25% to about 40%, of the phosphoprotein mass in the composition. In some embodiments, the composition includes the alpha s1 casein in an amount, by weight, of about 0%, 5%, 10%, 15%, 20%, 25%, 26%, 27%, 28%, 29%, 30%, 31%, 32%, 33%, 34%, 35%, 36%, 37%, 38%, 39%, 40%, or an amount within a range defined by any two of the preceding values.

In some embodiments, the alpha s2 casein is a phosphoprotein associated with the gene name CSN1S2. The alpha s2 casein can be a CSN1S2 phosphoprotein from any suitable mammal. In some embodiments, the alpha s2 casein is bovine (Gene ID: 282209), porcine (Gene ID: 445515), equine (Gene ID: 100327035), ovine (Gene ID: 443383), caprine (Gene ID: 100861229), or cameline (Gene ID: 105090951). In some embodiments, the alpha s2 casein is a polypeptide having an amino acid sequence at least 80%, at least 85%, at least 90%, at least 95%, at least 98%, or about 100% identical to the sequence set forth in SEQ ID NO: 18.

The composition can include any suitable amount of alpha s2 casein. In some embodiments, the composition includes the alpha s2 casein in an amount, by weight, between about 0% to about 20%, e.g., between about 2% to about 18%, between about 3% to about 18%, between about 4% to about 17%, between about 5% to about 16%, including between about 5% to about 15%, of the phosphoprotein mass in the composition. In some embodiments, the composition includes the alpha s2 casein in an amount, by weight, of about 0%, 1%, 2%, 3%, 4%, 5%, 6%, 7%, 8%, 9%, 10%, 11%, 12%, 13%, 14%, 15%, 18%, 20%, or an amount within a range defined by any two of the preceding values.

In some embodiments, the beta casein is a phosphoprotein associated with the gene name CSN2. The beta casein can be a CSN2 phosphoprotein from any suitable mammal. In some embodiments, the beta casein is bovine (Gene ID: 281099), porcine (Gene ID: 404088), equine (Gene ID: 100033903), ovine (Gene ID: 443391), caprine (Gene ID: 100860784), cameline (Gene ID: 105080412), or human (Gene ID: 1447). In some embodiments, the beta casein is a non-human beta casein. In some embodiments, the beta casein is a polypeptide having an amino acid sequence at least 80%, at least 85%, at least 90%, at least 95%, at least 98%, or about 100% identical to the sequence set forth in SEQ ID NO: 19 or 20.

The composition can include any suitable amount of beta casein. In some embodiments, the composition includes the beta casein in an amount, by weight, between about 0% to about 50%, e.g., between about 5% to about 50%, between about 10% to about 50%, between about 15% to about 45%, between about 20% to about 45%, including between about 25% to about 40%, of the phosphoprotein mass in the composition. In some embodiments, the composition includes the beta casein in an amount, by weight, of about 0%, 5%, 10%, 15%, 20%, 25%, 26%, 27%, 28%, 29%, 30%, 31%, 32%, 33%, 34%, 35%, 36%, 37%, 38%, 39%, 40%, or an amount within a range defined by any two of the preceding values.

In some embodiments, the kappa casein is a phosphoprotein associated with the gene name CSN3. The beta casein can be a CSN3 phosphoprotein from any suitable mammal. In some embodiments, the kappa casein is bovine (Gene ID: 281728), porcine (Gene ID: 445511), equine (Gene ID: 100033983), ovine (Gene ID: 443394), caprine (Gene ID: 100861231), cameline (Gene IDs: 105080408 or 105090949), or human (Gene ID: 1448). In some embodiments, the kappa casein is a non-human kappa casein. In some embodiments, the kappa casein is a polypeptide having an amino acid sequence at least 80%, at least 85%, at least 90%, at least 95%, at least 98%, or about 100% identical to the sequence set forth in SEQ ID NO: 21.

The composition can include any suitable amount of kappa casein. In some embodiments, the composition includes the kappa casein in an amount, by weight, between about 0% to about 20%, e.g., between about 2% to about 18%, between about 3% to about 18%, between about 4% to about 17%, between about 5% to about 16%, including between about 5% to about 15%, of the phosphoprotein mass in the composition. In some embodiments, the composition includes the kappa casein in an amount, by weight, of about 0%, 1%, 2%, 3%, 4%, 5%, 6%, 7%, 8%, 9%, 10%, 11%, 12%, 13%, 14%, 15%, 18%, 20%, or an amount within a range defined by any two of the preceding values.

Combinations of caseins from different species are used, in some embodiments. For example, in several embodiments, one or more human casein is used in combination with one or more bovine casein. Ratios of caseins are used in some embodiments, for example a 3:1:3:1 ratio of alpha S1 casein:alpha s2 casein:beta casein:kappa casein. Different ratios may be used in some embodiments, for example 4:1:4:1, 2:1:2:1, or 1:1:1:1. Ratios may also be used between any two given caseins in a composition, ranging from 1:1, 2:1, 3:1, 4:1, 5:1, 10:1, 1:5, 1:4, 1:3, 1:2, etc.

Any suitable total amount of the phosphoproteins may be present in the composition. In some embodiments, the phosphoproteins are present in an amount between 5% to about 10%, e.g., about 6% to about 10%, about 6% to about 9%, including about 6% to about 8%, (weight by volume) of the composition. In some embodiments, the phosphoproteins are present in an amount of about 5%, 6%, 7%, 8%, 9%, 10%, or an amount within a range defined by any two of the preceding values, (weight by volume) of the composition.

In some embodiments, one or more of the casein phosphoproteins are non-human casein phosphoproteins. In some embodiments, the exosomes and at least one of the casein phosphoproteins are from different species. In some embodiments, the exosomes are human exosomes, and one or more of the casein phosphoproteins are non-human casein phosphoproteins. In some embodiments, the exosomes are human exosomes, and one or more of the casein phosphoproteins are bovine (or ovine, porcine, caprine, cameline, or equine) casein phosphoproteins.

In some embodiments, the composition includes micellar structures formed by at least a portion of the casein phosphoproteins. In some embodiments, the casein micelles are substantially spherical. In some embodiments, a casein micelle in the composition has an average diameter (as measured per micelle) of about 40 nm, about 50 nm, about 60 nm, about 70 nm, about 80 nm, about 90 nm, about 100 nm, about 110 nm, about 120 nm, about 130 nm, about 150 nm, about 200 nm, about 250 nm, about 300 nm, about 350 nm, about 400 nm, about 450 nm, about 500 nm or more, or an average diameter within a range defined by any two of the preceding values. In some embodiments, a casein micelle in the composition has an average diameter (as measured per micelle) in a range from about 40 nm to about 500 nm, e.g., from about 40 nm to about 400 nm, from about 50 nm to about 300 nm, from about 60 nm to about 250 nm, from about 70 nm to about 250 nm, from about 80 nm to about 200 nm, including from about 90 nm to about 150 nm. The casein micelles of the present composition are generally not precipitated or in gel form.

In some embodiments, the composition includes one or more colloidal minerals (e.g., minerals in suspension). In several embodiments, a complex (e.g., two or more) minerals are used as a colloidal mineral complex. The colloidal mineral complex can include any suitable mineral compounds and/or their salts. In some embodiments, the colloidal mineral complex includes, without limitation, one or more of calcium, magnesium, inorganic phosphate, citrate, sodium, potassium, and chloride, or their respective salts. In some embodiments, the colloidal mineral complex is present in an amount between about 2% and about 15%, e.g., about 2% to about 12%, about 5% to about 10%, about 5% to about 9%, including about 6% to about 9% (by weight) of the phosphoprotein mass in the composition. In some embodiments, the colloidal mineral complex is present in an amount of about 2%, 3%, 4%, 5%, 6%, 7%, 8%, 9%, 10%, 11%, 12%, 13%, 14%, 15% or an amount within a range defined by any two of the preceding percentages.

In some embodiments, the composition is in a parenteral dose form. In some embodiments, the parenteral dosage form is sterile or capable of being sterilized before administering to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration to a subject. Suitable excipients that can be used to provide parenteral dosage forms of the nucleic acid include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

Also provided herein is a macrophage that includes a nucleic acid of the present disclosure. In some embodiments, the macrophage is a CD68+ macrophage. In some embodiments, the macrophage is a human macrophage. In some embodiments, a macrophage that has been exposed to the nucleic acid increases anti-inflammatory activity compared to a suitable control, e.g., a macrophage that has not been exposed to the nucleic acid, or a macrophage that has been exposed to a control nucleic acid. In some embodiments, the macrophage is a bone marrow-derived macrophage (BMDM). In some embodiments, the macrophage has increased expression of one or more of IL-10, IRF-7, NOS-2, and ARG-1. In some embodiments, the macrophage has increased mRNA expression of one or more of IL-10, IRF-7, NOS-2, and ARG-1, compared to a suitable control, e.g., a macrophage that has not been contacted with the nucleic acid. In some embodiments, the macrophage has increased mRNA expression of one or more of IL-10, IRF-7, NOS-2, and ARG-1, compared to a suitable control, e.g., a macrophage that has not been contacted with the nucleic acid. In some embodiments, mRNA expression of one or more of IL-10, IRF-7, NOS-2, and ARG-1 in the macrophage having the nucleic acid is each independently increased by at least 1.5 fold, 2 fold, 4 fold, 6 fold, 8 fold, 10 fold, 15 fold, 20 fold, 30 fold, 50 fold, 100 fold, 200 fold, 300 fold, 400 fold 500 fold, 1,000 fold, or more, or by a fold amount in a range defined by any two of the preceding values, compared to a suitable control, e.g., a macrophage that has not been contacted with the nucleic acid. In some embodiments, the macrophage has increased protein expression of one or more of IL-10, IRF-7, NOS-2, and ARG-1, compared to a suitable control, e.g., a macrophage that has not been contacted with the nucleic acid. In some embodiments, protein expression of one or more of IL-10, IRF-7, NOS-2, and ARG-1 in the macrophage having the nucleic acid is each independently increased by at least 1.5 fold, 2 fold, 4 fold, 6 fold, 8 fold, 10 fold, 15 fold, 20 fold, 30 fold, 50 fold, 100 fold, 200 fold, or more, or by a fold amount in a range defined by any two of the preceding values, compared to a suitable control, e.g., a macrophage that has not been contacted with the nucleic acid. In some embodiments, the macrophage has increased secretion of IL-10 compared to a suitable control, e.g., a macrophage that has not been contacted with the nucleic acid. In some embodiments, IL-10 secretion in the macrophage having the nucleic acid is each independently increased by at least about 1.2 fold, 1.5 fold, 2 fold, 2.5 fold, 3 fold, 4 fold, 5 fold, 6 fold, 8 fold, 10 fold, 15 fold, 20 fold, 50 fold, or more, or by a fold amount in a range defined by any two of the preceding values, compared to a suitable control, e.g., a macrophage that has not been contacted with the nucleic acid. In some embodiments, the macrophage is in culture. In some embodiments, the macrophage is in a subject, e.g., in peripheral blood, bone marrow, and/or at a site of tissue injury.

### Methods

Provided herein are methods of treating a subject in need thereof using the nucleic acids of the present disclosure (also referred to herein as “treatment methods”). Conditions that may be treated by the treatment methods include, without limitation, heart conditions and inflammatory conditions. In some embodiments, the conditions include, without limitation, muscular disorders, myocardial infarction, cardiac disorders, myocardial alterations, muscular dystrophy, fibrotic disease, inflammatory disease, viral infection, sepsis or wound healing. In some embodiments, conditions treated by the treatment methods include, without limitation, conditions associated with inflammation and/or fibrosis. In some embodiments, a subject treated by administering the nucleic acids of the present disclosure, according to the treatment methods herein, are in need of treatment for conditions associated with inflammation and/or fibrosis. The conditions associated with inflammation and/or fibrosis can include, without limitation, inflammation and/or fibrosis of the heart, skeletal muscle, or skin. In some embodiments, the conditions associated with inflammation and/or fibrosis includes aging. In some embodiments, one or more symptoms of inflammation and/or fibrosis associated with aging are treated or reversed by administering the nucleic acids of the present disclosure according to the treatment methods herein. In some embodiment, a subject suffering from accelerated aging (e.g., progeria) is treated by administering the nucleic acids of the present disclosure according to the treatment methods herein. In some embodiments, the conditions treated by the present treatment methods are a symptom and/or sequelae of an infection. In some embodiments, the infection is a viral infection, e.g., a respiratory virus infection, such as COVID-19, infections due to other coronaviruses, or other viral pathogens (e.g., flu, HIN1, Hepatitis C, HIV, etc.).

In some embodiments, a treatment method includes a method of treating a muscle disorder (or muscle condition) or symptom thereof, the method including administering to a subject in need of treating a muscle disorder or symptom thereof a therapeutically effective amount of the nucleic acid (or the composition containing the nucleic acid) of the present disclosure. The muscle disorder can be, without limitation, a skeletal muscle disorder or a cardiac muscle disorder. In some embodiments, the muscle disorder includes muscular dystrophy, e.g., Duchenne muscular dystrophy. In some embodiments, the subject has muscular dystrophy, or is at risk of developing muscular dystrophy. In some embodiments, the subject is genetically predisposed to developing muscular dystrophy, e.g., Duchenne muscular dystrophy. In some embodiments, the subject has one or more mutations in a dystrophin gene that predisposes the subject to developing muscular dystrophy, e.g., Duchenne muscular dystrophy.

In some embodiments, a treatment method includes a method of treating a skin condition, e.g., an inflammation and/or fibrosis of the skin (such as, but not limited to, scleroderma). In some embodiments, the method includes administering to a subject in need of treating an inflammation and/or fibrosis of the skin a therapeutically effective amount of the nucleic acid (or the composition containing the nucleic acid) of the present disclosure. In some embodiments, the inflammation and/or fibrosis of the skin is scleroderma.

In some embodiments, a treatment method includes a method of treating a heart condition or symptom thereof, the method including administering to a subject in need of treating a heart condition or symptom thereof a therapeutically effective amount of the nucleic acid (or the composition containing the nucleic acid) of the present disclosure. In some embodiments, the subject is a human subject. In some embodiments, the subject is a non-human subject, e.g., a non-human mammal.

A variety of heart conditions may be treated by the present method. In some embodiments, the heart condition includes a symptom and/or sequelae of heart failure or myocardial infarction. In some embodiments, the heart condition includes hypertrophic cardiomyopathy. In some embodiments, the heart condition includes heart failure with preserved ejection fraction (HFpEF).

In some embodiments, the subject is at risk of developing the heart condition. In some embodiments, the subject is at risk of developing the heart condition based on one or more of the subject's family history, genetic predisposition, life style, and medical history. In some embodiments, the subject has a mutation in cardiac troponin I that predisposes the subject to developing hypertrophic cardiomyopathy (HCM). In some embodiments, the subject has one or more comorbidities for the heart condition. In some embodiments, the one or more comorbidities includes obesity and hypertension. In some embodiments, the subject has, or is diagnosed with, the heart condition.

In some embodiments, the subject exhibits one or more of: hypertension, elevated E/e′ ratio, cardiac hypertrophy, myocardial fibrosis, obesity, wasting, reduced endurance, and elevated systemic inflammatory markers. In some embodiment, the subject has hypertension, and administering the therapeutically effective amount of the nucleic acid (or composition thereof) reduces the subject's blood pressure. In some embodiments, a subject having hypertension has a resting blood pressure of over 130/90 mmHg. In some embodiments, a subject having hypertension has a resting blood pressure of over 140/90 mmHg. In some embodiment, administering the therapeutically effective amount of the nucleic acid (or composition thereof) reduces the subject's systolic blood pressure or diastolic blood pressure. In some embodiment, the subject's blood pressure (systolic or diastolic blood pressure) is reduced by at least about 5%, 7.5%, 10%, 12.5%, 15%, 17.5%, 20%, 25%, 30% or more, or by a percentage in a range defined by any two of the preceding values, after administering the therapeutically effective amount of the nucleic acid (or composition thereof). In some embodiment, the subject's blood pressure (systolic or diastolic blood pressure) is reduced at least to a level that is deemed no longer to be hypertensive after administering the therapeutically effective amount of the nucleic acid (or composition thereof).

In some embodiment, the subject has an elevated E/e′ ratio, and administering the therapeutically effective amount of the nucleic acid (or composition thereof) reduces the E/e′ ratio. In some embodiment, the subject's E/e′ ratio is reduced by at least about 5%, 7.5%, 10%, 12.5%, 15%, 17.5%, 20%, 25%, 30%, 35%, 40%, 45%, 50%, 55%, 60%, 65%, 70%, 75% or more, or by a percentage in a range defined by any two of the preceding values, after administering the therapeutically effective amount of the nucleic acid (or composition thereof). In some embodiment, the subject's E/e′ ratio is reduced at least to a level that is deemed no longer to be clinically relevant after administering the therapeutically effective amount of the nucleic acid (or composition thereof).

In some embodiment, the subject has cardiac hypertrophy, and administering the therapeutically effective amount of the nucleic acid (or composition thereof) reduces cardiac hypertrophy. Cardiac hypertrophy can be measured using any suitable option. In some embodiments, cardiac hypertrophy is measured using echocardiography. In some embodiments, a subject having cardiac hypertrophy has an increased diastolic interventricular septal wall diameter (IVSd) and/or left ventricular posterior wall diameter (LVPWd), as measured by echocardiography, and administering the therapeutically effective amount of the nucleic acid (or composition thereof) reduces the IVSd and/or LVPWd. In some embodiment, the subject's IVSd or LVPWd is reduced by at least about 5%, 7.5%, 10%, 12.5%, 15%, 17.5%, 20%, 25%, 30% or more, or by a percentage in a range defined by any two of the preceding values, after administering the therapeutically effective amount of the nucleic acid (or composition thereof). In some embodiment, the subject's IVSd or LVPWd is reduced at least to a level that is deemed no longer to be hypertrophic after administering the therapeutically effective amount of the nucleic acid (or composition thereof).

In some embodiment, the subject has myocardial fibrosis, and administering the therapeutically effective amount of the nucleic acid (or composition thereof) reduces cardiac fibrosis. “Fibrosis” as used herein can include any remodeling (e.g., pathological remodeling) of tissue (e.g., the myocardium), such as, but not limited to, deposition of fibrotic and/or fatty tissue, replacement of muscle tissue with fibrotic and/or fatty tissue, etc. Cardiac fibrosis is monitored using any suitable means, such as biopsy, ultrasonography or MRI. In some embodiments, administering the therapeutically effective amount of the nucleic acid (or composition thereof) eliminates or retards the development of myocardial fibrosis.

In some embodiments, the subject has inflammation and/or fibrosis associated with an autoimmune condition. In some embodiments, the subject has scleroderma or systemic sclerosis.

In some embodiment, the subject exhibits wasting or weight loss, and administering the therapeutically effective amount of the nucleic acid (or composition thereof) retards or prevents the wasting. In some embodiment, the subject exhibits body weight loss of at most about 20%, 15%, 10%, 5%, 3% or less, or a percentage in a range defined by any two of the preceding values, after administering the therapeutically effective amount of the nucleic acid (or composition thereof). In some embodiment, the subject's body weight recovers to, or is maintained at substantially the pre-treatment level after administering the therapeutically effective amount of the nucleic acid (or composition thereof).

In some embodiment, the subject exhibits reduced endurance, e.g., exercise endurance, and administering the therapeutically effective amount of the nucleic acid (or composition thereof) retards or prevents the decline in endurance. In some embodiment, the subject exhibits a decline in endurance of at most about 20%, 15%, 10%, 5%, 3% or less, or a percentage in a range defined by any two of the preceding values, after administering the therapeutically effective amount of the nucleic acid (or composition thereof). In some embodiment, the subject's exercise endurance recovers to, or is maintained at substantially the pre-treatment level after administering the therapeutically effective amount of the nucleic acid (or composition thereof). In some embodiment, the subject exhibits an improvement in endurance of at least about 5%, 10%, 15%, 20%, 25%, 30% 35%, 40%, 50% or more, or a percentage in a range defined by any two of the preceding values, after administering the therapeutically effective amount of the nucleic acid (or composition thereof). In some embodiments, the improvement in endurance after administering the therapeutically effective amount of the nucleic acid (or composition thereof) is sustained over the duration of treatment. In some embodiments, the improvement in endurance after administering the therapeutically effective amount of the nucleic acid (or composition thereof) is sustained across multiple doses of administration.

In some embodiments, the subject exhibits elevated levels of systemic inflammatory markers, e.g., in the peripheral blood. In some embodiments, the systemic inflammatory marker includes one or more of IL-6 and brain natriuretic peptide (BNP). In some embodiments, the level of the systemic inflammatory marker is reduced by at least about 5%, 7.5%, 10%, 12.5%, 15%, 17.5%, 20%, 25%, 30%, 35%, 40%, 45%, 50%, 55%, 60%, 65%, 70%, 75% or more, or by a percentage in a range defined by any two of the preceding values, after administering the therapeutically effective amount of the nucleic acid (or composition thereof). In some embodiment, the subject's systemic inflammatory marker is reduced at least to a level that is deemed no longer to be elevated after administering the therapeutically effective amount of the nucleic acid (or composition thereof).

In some embodiments, where the subject is obese, the therapeutic effect of administering the nucleic acid is independent of the subject's obesity. In some embodiments, administering the nucleic acid (or composition thereof) does not affect the subject's weight.

In some embodiments, the subject exhibits reduced skeletal muscle function, e.g., the amount of force or torque exerted by a skeletal muscle group. In some embodiments, the subject exhibits reduced skeletal muscle function and administering the therapeutically effective amount of the nucleic acid (or composition thereof) retards the development of reduced skeletal muscle function, prevents deterioration of skeletal muscle function, or enhances skeletal muscle function. In some embodiment, the subject's skeletal muscle function recovers to, or is maintained at substantially the pre-treatment level after administering the therapeutically effective amount of the nucleic acid (or composition thereof). In some embodiment, the subject exhibits an improvement in skeletal muscle function of at least about 5%, 10%, 15%, 20%, 25%, 30% 35%, 40%, 50% or more, or a percentage in a range defined by any two of the preceding values, after administering the therapeutically effective amount of the nucleic acid (or composition thereof).

In some embodiments, any of the therapeutic effects of administering the therapeutically effective amount of the nucleic acid (or composition thereof) herein is sustained over the duration of treatment. is sustained across multiple doses of administration is sustained across multiple doses of administration. In some embodiments, any of the therapeutic effects of administering the therapeutically effective amount of the nucleic acid (or composition thereof) herein is not transient over the duration of treatment.

In some embodiments, a treatment method of the present disclosure treats any one or more of a variety of inflammatory conditions. In some embodiments, the inflammatory condition is a chronic condition. In some embodiments, the inflammatory condition is one that is responsive to the anti-inflammatory effect of IL-10. In some embodiments, the inflammatory condition includes an autoimmune disease, graft-versus-host disease (GVHD) or an immune response to an organ transplant. In some embodiments, the inflammatory condition includes viral infection, sepsis, arthritis (rheumatoid arthritis, juvenile rheumatoid arthritis, psoriatic arthritis), multiple sclerosis, pemphigus, and type 1 diabetes (also referred to as insulin-dependent diabetes mellitus (IDDM)). In some embodiments, the inflammatory condition includes Behçet's disease, polymyositis/dermatomyositis, autoimmune cytopenias, autoimmune myocarditis, primary liver cirrhosis, Goodpasture's syndrome, autoimmune meningitis, Sjögren's syndrome, systemic lupus erythematosus, Addison's disease, alopecia greata, ankylosing spondylitis, autoimmune hepatitis, autoimmune mumps, Crohn's disease, insulin-dependent diabetes mellitus, dystrophic epidermolysis bullosa, epididymitis, glomerulonephritis, Graves' disease, Guillain-Barré syndrome, Hashimoto's disease, hemolytic anemia, multiple sclerosis, myasthenia gravis, pemphigus vulgaris, psoriasis, rheumatic fever, rheumatoid arthritis, sarcoidosis, scleroma, spondyloarthropathy, thyroiditis, vasculitis, vitiligo, myxedema, pernicious anemia and ulcerative colitis. In some embodiments, the inflammation is related to a bone marrow transplantation. In some embodiments, the inflammation is related to allograft rejection following tissue transplantation. In some embodiments, the autoimmune disease is a cardiac autoimmune disease, e.g., autoimmune myocarditis. In some embodiments, the autoimmune disease is scleroderma or systemic sclerosis.

In some embodiments, a treatment method of the present disclosure treats symptoms and/or sequelae of any one or more of a variety of infectious diseases. In some embodiments, a heart condition or inflammatory condition treated by the nucleic acids of the present disclosure includes a symptom and/or sequelae of an infectious disease. In some embodiments, the infectious disease is associated with myocardial injury. In some embodiments, the heart condition includes acute myocarditis associated with the infectious disease. In some embodiment, the inflammatory condition includes a cytokine storm, or hyperinflammation, associate with the infectious disease. In some embodiments, the inflammatory condition includes acute lung injury or acute respiratory distress syndrome (ARDS).

In some embodiments, the infectious disease is an infection by, without limitation, one or more of the following pathogens: viruses (including but not limited to coronavirus, human immunodeficiency virus, herpes simplex virus, papilloma virus, parainfluenza virus, influenza virus, hepatitis virus, Coxsackie Virus, herpes zoster virus, measles virus, mumps virus, rubella, rabies virus, hemorrhagic viral fevers, HIN1, and the like), prions, parasites, fungi, mold, yeast and bacteria (both gram-positive and gram-negative). In some embodiments, pathogens include, without limitation, Candida albicans, Aspergillus niger, Escherichia coli (E. coli), Pseudomonas aeruginosa (P. aeruginosa), and Staphylococcus aureus (S. aureus), Group A streptococci, S. pneumoniae, Mycobacterium tuberculosis, Campylobacter jejuni, Salmonella, Shigella, and a variety of drug resistant bacteria.

In some embodiments, the inflammation is subsequent to or concurrent with an infection by a virus, e.g., a DNA or RNA virus. In some embodiments, the virus is an RNA virus, e.g., a single or double-stranded virus. In some embodiments, the RNA virus is a positive sense, single-stranded RNA virus. In some embodiments, the virus belongs to the Nidovirales order. In some embodiments, the virus belongs to the Coronaviridae family. In some embodiments, the virus belongs to the alphacoronavirus, betacoronavirus, gammacoronavirus or deltacoronavirus genus. In some embodiments, the alphacoronavirus is, without limitation, human coronavirus 229E, human coronavirus NL63 or transmissible gastroenteritis virus (TGEV). In some embodiments, the betacoronavirus is, without limitation, Severe Acute Respiratory Syndrome Coronavirus (SARS-CoV), SARS-CoV-2 (COVID-19), Middle Eastern Respiratory Syndrome Coronavirus (MERS-CoV), human coronavirus HKU1, or human coronavirus OC43. In some embodiments, the gammacoronavirus is infectious bronchitis virus (IBV).

The nucleic acid can be administered to the subject at any suitable amount. In some embodiments, the therapeutically effective amount of the nucleic acid includes about 0.01 μg, 0.02 μg, 0.05 μg, 0.1 μg, 0.2 μg, 0.5 μg, 1 μg, 2 μg, 3 μg, 4 μg, 5 μg, 6 μg, 7 μg, 8 μg, 9 μg, 10 μg, 15 μg, 20 μg, 25 μg, 30 μg, 40 μg, 50 μg, 75 μg, 100 μg, 125 μg, 150 μg, 175 μg, 200 μg, 250 μg, 300 μg, 400 μg, 500 μg, 600 μg, 700 μg, 800 μg, 900 μg, 1 mg, 2 mg, 3 mg, 4 mg, 5 mg, 10 mg, 15 mg, 20 mg, 30 mg, 40 mg, 50 mg, 75 mg, 100 mg or more, or an amount in a range defined by any two of the preceding values (e.g., 0.01 μg-0.1 μg, 0.1 μg-1 μg, 1 μg-10 μg, 10 μg-100 μg, 100 μg-1 mg, 1 mg-10 mg, 10 mg-100 mg). In some embodiments, the therapeutically effective amount of the nucleic acid includes about 0.001 μg/g, 0.002 μg/g, 0.005 μg/g, 0.01 μg/g, 0.02 μg/g, 0.05 μg/g, 0.1 μg/g, 0.15 μg/g, 0.2 μg/g, 0.5 μg/g, 1 μg/g, 2 μg/g, 3 μg/g, 4 μg/g, 5 μg/g, 6 μg/g, 7 μg/g, 8 μg/g, 9 μg/g, 10 μg/g, 15 μg/g, 20 μg/g, 25 μg/g, 30 μg/g, 35 μg/g, 40 μg/g, 45 μg/g, 50 μg/g, 60 μg/g, 70 μg/g, 80 μg/g, 90 μg/g, 100 μg/g of body weight, or more, or an amount in a range defined by any two of the preceding values (e.g., 0.001 μg/g-0.01 μg/g, 0.01 μg/g-0.1 μg/g, 0.1 μg/g-1 μg/g, 1 μg/g-10 μg/g, 10 μg/g-100 μg/g). In some embodiments, the therapeutically effective amount of the nucleic acid is about 0.001 μg/g, 0.002 μg/g, 0.005 μg/g, 0.01 μg/g, 0.02 μg/g, 0.05 μg/g, 0.1 μg/g, 0.2 μg/g, 0.5 μg/g, or about 1 μg/g of body weight, or more, or an amount in a range defined by any two of the preceding values (e.g., 0.001 μg/g-0.01 μg/g, 0.01 μg/g-0.05 μg/g, 0.05 μg/g-0.1 μg/g, 0.1 μg/g-0.2 μg/g, 0.2 μg/g-0.5 μg/g, or 0.5 μg/g-1 μg/g).

The nucleic acid or composition can be administered to the subject at any suitable dosing schedule. In some embodiments, the therapeutically effective amount of the nucleic acid or the composition is administered to the subject no more frequently than twice a week, once a week, once every two weeks, once every month, once every two months, once every three months, once every four months or longer, or at a frequency in a range defined by any two of the preceding values. In some embodiments, the nucleic acid is administered to the subject 1, 2, 3, 4, 5, 6, 7, 8, 9, 10, 15, 20, 25, 30 or more times. In some embodiments, the nucleic acid is administered to the subject at regular intervals.

The nucleic acid or composition can be administered using any suitable route. Administration can be local or systemic. In some embodiments, administration is parenteral. Suitable option for administration include, without limitation, intravenous, intramuscular, subcutaneous, intra-arterial, intraperitoneal, or oral administration. In some embodiments, the nucleic acid or composition is administered intravenously. In some embodiments, the nucleic acid or composition is administered by infusion.

Also provided herein is a method of promoting anti-inflammatory activity of macrophages (also referred to herein as macrophage-modulating methods). The method generally includes contacting the nucleic acid or the composition of the present disclosure with a population of macrophages. In some embodiments, the nucleic acid induces changes in gene expression and/or epigenetic changes in macrophages that are exposed to the nucleic acids. In some embodiments, contacting the nucleic acid (or composition) increases expression of one or more of IL-10, IRF-7, NOS-2, and ARG-1. In some embodiments, contacting the nucleic acid (or composition) increases transcription or translation of one or more of IL-10, IRF-7, NOS-2, and ARG-1. In some embodiments, contacting the nucleic acid (or composition) increases transcription of one or more of IL-10, IRF-7, NOS-2, and ARG-1 each independently by at least about 1.5 fold, 2 fold, 4 fold, 6 fold, 8 fold, 10 fold, 15 fold, 20 fold, 30 fold, 50 fold, 100 fold, 200 fold, 300 fold, 400 fold 500 fold, 1,000 fold, or more, or by a fold amount in a range defined by any two of the preceding values, compared to a suitable control, e.g., a macrophage that has not been contacted with the nucleic acid.

In some embodiments, contacting the nucleic acid (or composition) increases secretion of interleukin 10 (IL-10) in the macrophages. In some embodiments, contacting the nucleic acid (or composition) increases secretion of IL-10 from the macrophages by at least about 1.2 fold, 1.5 fold, 2 fold, 2.5 fold, 3 fold, 4 fold, 5 fold, 6 fold, 8 fold, 10 fold, 15 fold, 20 fold, 50 fold, or more, or by a fold amount in a range defined by any two of the preceding values, compared to a suitable control, e.g., a macrophage that has not been contacted with the nucleic acid.

The population of macrophages can be contacted with the nucleic acid or composition for any suitable amount of time. In some embodiments, the contacting is for 24 hours or more, 36 hours or more, 48 hours or more, 60 hours or more, 72 hours or more, or an amount of time in a range between any two of the preceding values.

In some embodiments, the contacting is done in vitro. In some embodiments, the contacting is done in vivo. In some embodiments, the contacting includes administering to a subject in need of treating an inflammation an effective amount of the nucleic acid or the composition. In some embodiments, the macrophage is a human macrophage. In some embodiments, the subject is a human subject. In some embodiments, the subject is a non-human subject, e.g., a non-human mammal.

Any suitable amount of nucleic acid can be contacted with the population of macrophages to promote the anti-inflammatory activity of the macrophages. In some embodiments, the effective amount depends on whether the contacting is done in vivo or in vitro.

In any of the treatment methods, the method can include administering a nucleic acid that specifically binds Translocated Promoter Region (TPR). In any of the treatment methods, in some embodiments, the method can include administering a nucleic acid that specifically binds TPR. In some embodiments, TPR is human TPR (Gene ID 7175). In some embodiments, TPR has the amino acid sequence shown below, or an amino acid sequence at least 80%, 85%, 90%, 95%, 97%, 99% identical thereto:

| (SEQ ID NO: 16) | |
|---|---|
| 1 | maavlqqvle rtelnklpks vqnklekfla dqqseidglk grhekfkves eqqyfeiekr |
| 61 | lshsqerlvn etrecqslrl elekInnqlk alteknkele iaqdrniaiq sqftrtkeel |
| 121 | eaekrdlirt nerlsqeley ltedvkrlne klkesnttkg elqlkldelq asdvsvkyre |
| 181 | krlegekell hsqntwInte lktktdella lgrekgneil elkonlenkk eevsrleeqm |
| 241 | nglktsnehl qkhvedlltk lkeakeqqas meekfhneln ahiklsnlyk saaddseaks |
| 301 | neltraveel hkllkeagea nkaiqdhlle veqskdqmek emlekigrle kelenandll |
| 361 | satkrkgail seeelaamsp taaavakivk pgmkltelyn ayvetqdqll leklenkrin |
| 421 | kyldeivkev eakapilkrq reeyeraqka vaslsvkleq amkeiqrlqe dtdkankqss |
| 481 | vlerdnrrme iqvkdlsqqi rvllmeleea rgnhvirdee vssadissss evisqhlvsy |
| 541 | rnieelqqqn qrllvalrel getrereeqe ttsskitelq lklesaltel eqlrksrqhq |
| 601 | mqlvdsivrq rdmyrillsq ttgvaiplha sslddvslas tpkrpstsqt vstpapvpvi |
| 661 | esteaieaka alkqlqeife nykkekaene kiqneqlekl qeqvtdlrsq ntkistqldf |
| 721 | askryemlqd nvegyrreit slhernqklt attqkqeqii ntmtqdlrga neklavaevr |
| 781 | aenlkkekem lklsevrlsq qresllaeqr gqnllltnlq tiqgilerse tetkqrlssq |
| 841 | iekleheish lkkkleneve qrhtltrnld vqlldtkrql dtetnlhlnt kellknaqke |
| 901 | iatlkqhlsn mevqvasqss qrtgkgqpsn kedvddlvsq lrqteeqvnd lkerlktsts |
| 961 | nveqyqamvt sleeslnkek qvteevrkni evrlkesaef qtqlekklme vekekqelqd |
| 1021 | dkrraiesme qqlselkktl ssvqnevqea lqrastalsn eqqarrdcqe qakiaveaqn |
| 1081 | kyerelmlha advealqaak eqvskmasvr qhleettqka esqlleckas weerermlkd |
| 1141 | evskcvcrce dlekqnrllh dqieklsdkv vasvkegvqg plnvslseeg ksqeqileil |
| 1201 | rfirrekeia etrfevaqve slryrqrvel lerelgelqd slnaerekvq vtaktmaqhe |
| 1261 | elmkktetmn vvmetnkmlr eekerleqdl qqmqakvrkl eldilplqea naelseksgm |
| 1321 | lqaekkllee dvkrwkarnq hlvsqqkdpd teeyrkllse kevhtkriqq lteeigrlka |
| 1381 | eiarsnaslt nnqnliqslk edlnkvrtek etiqkdldak iidiqekvkt itqvkkigrr |
| 1441 | yktqyeelka qqdkvmetsa qssgdhqeqh vsvqemqelk etlnqaetks kslesqvenl |
| 1501 | qktlsekete arnlqeqtvq lqselsrlrq dlqdrttqee qlrqqiteke ektrkaivaa |
| 1561 | kskiahlagv kdqltkenee lkqrngaldq qkdeldvrit alksqyegri srlerelreh |
| 1621 | qerhleqrde pqepsnkvpe qqrqitlktt pasgergias tsdpptanik ptpvvstpsk |
| 1681 | vtaaamagnk stprasirpm vtpatvtnpt ttptatvmpt tqvesqeamq segpvehvpv |
| 1741 | fgstsgsvrs tspnvqpsis qpiltvqqqt qatafvqptq qshpqiepan qelssnivev |
| 1801 | vqsspverps tstavfgtvs atpssslpkr treeeedsti easdqvsddt vemplpkklk |
| 1861 | svtpvgteee vmaeestdge vetqvynqds qdsigegvtq gdytpmedse etsqslqidl |
| 1921 | gplqsdqqtt tssqdgqgkg ddvividsdd eeedddendg ehedyeedee dddddeddtg |
| 1981 | mgdegedsne gtgsadgndg yeaddaeggd gtdpgtetee smgggegnhr aadsqnsgeg |
| 2041 | ntgaaessfs qevsreqqps saserqapra pqsprrpphp lpprltihap pqelgppvqr |
| 2101 | iqmtrrqsvg rglqltpgig gmqqhffdde drtvpstptl vvphrtdgfa eaihspqvag |
| 2161 | vprfrfgppe dmpqtssshs dlgqlasqgg lgmyetplfl aheeesggrs vpttplqvaa |
| 2221 | pvtvftestt sdasehasqs vpmvttstgt lsttnetatg ddgdevfvea esegisseag |
| 2281 | leidsqqeee pvqasdesdl pstsqdppss ssvdtsssqp kpfrrvrlqt tlrqgvrgrq |
| 2341 | fnrqrgvsha mggrgginrg nin |

In some embodiments, a TPR-binding nucleic acid binds to TPR with a binding affinity of 10⁻⁵M-10⁻¹²M, e.g., 10⁻⁶M to 10⁻¹¹M. In some embodiments, the nucleic acid that specifically binds TPR inhibits or reduces function and/or expression of TPR. In some embodiments, the nucleic acid that specifically binds TPR is an RNA. In some embodiments, the nucleic acid that specifically binds TPR is a non-coding RNA. In some embodiments, the nucleic acid that specifically binds TPR is a Y RNA, or a derivative thereof, e.g., a synthetic derivative thereof. In some embodiments, the nucleic acid that specifically binds TPR is any one of the nucleic acids of the present disclosure derived from EV-YF1, e.g., TY4.

### Kits

Also provided herein are kits that include the nucleic acid or a composition of the present disclosure. The present kit in some embodiments finds use in treating a muscle disorder, a heart condition or an inflammatory condition (e.g., associated with a viral infection), as provided herein. A kit can include the nucleic acid of the present disclosure and a transfection reagent. The transfections reagent can be any suitable transfection reagent, as provided herein. In some embodiments, the transfection reagent includes one or more of a lipid (e.g., a liposome-forming lipid), a PEGylated lipid, and an extracellular vesicle. In some embodiments, the kit includes a pharmaceutically acceptable excipient, as provided herein. In some embodiments, the kit includes casein and/or chitosan. Kits can include one or more containers (e.g., vials, ampoules, test tubes, flasks or bottles) for holding one or more components of the kits. The kits may further include instructions for using the kit to treat a condition (e.g., HCM, HFpEF, muscular dystrophy, scleroderma, inflammatory condition associated with a viral infection). The information and instructions may be in the form of words, pictures, or both, and the like.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

### EXAMPLES

### Example 1

This non-limiting example shows a potential mechanism of action for the bioactivity of non-coding RNA (ncRNA) derived from CDC-derived extracellular vesicles (CDC-EV).

EV-YF1 (SEQ ID NO: 1; FIG. 1A) is a 56-nucleotide (nt) ncRNA found in CDC-EV, and is encoded by the human Y-RNA4 gene. EV-YF1 increases secretion of IL-10 (an anti-inflammatory cytokine) by macrophages and is cardioprotective against myocardial infarction (MI). EV-YF1 exerts antifibrotic and anti-hypertrophic benefits comparable to those of CDc-EVs in a model of hypertension and hypertrophy induced by angiotensin II infusion. EV-YF1 and its derivatives also show strong bioactivity in a model of HFpEF.

Macrophages as disease-relevant targets of EVs. As crucial players in innate immunity, macrophages secrete inflammatory mediators, scavenge cellular debris (by efferocytosis), and remodel tissues after injury. Macrophages were identified as key effectors of post-MI cardioprotection induced by CDC-EV and implicated enhanced efferocytosis in the mechanism. Further verification of the central role of macrophages in heart failure (HF) comes from findings that macrophage depletion not only undermines cardioprotection but also exacerbates murine HFpEF as well as mdx cardiomyopathy. The emerging concept is that macrophages are not only EV-modulated “first responders” but also robust, disease-relevant in vitro reporters of EV bioactivity. Rat bone marrow-derived macrophages (BMDM) can be used to study the effects of EV-YF1 and CDC-EV on the transcriptome and epigenome, respectively. Macrophages may be important modulators of heart failure (HF), and CDC-EV dramatically alter macrophage phenotype.

### Example 2

This non-limiting example shows the effect of EV-YF1 on target cell transcriptome and the therapeutic effect of EV-YF1 in a model of hypertrophic cardiomyopathy (HCM).

Y-RNA effects on gene expression and epigenomics. FIG. 1A depicts a working hypothesis that Y-RNA derivatives induce epigenetic modifications which alter gene expression in target cells. Epigenetic regulation refers to chromatin adaptations or DNA methylations which establish long-lasting gene expression patterns. Indeed, EV-YF1 induces major transcriptomic changes in rat bone marrow-derived macrophages (BMDM) (FIG. 1B), which include enhanced IL10 expression (FIG. 1C). One distinctive feature of CDC-EV effects (and perhaps those of EV-YF1) is their persistence, which might be explained by epigenetic modulation. FIG. 1D shows H3K27ac marks (an epigenetic modification of histone H3) at the IL10 locus, 3 of which are induced by exposure to CDc-EVs. H3K27ac defines active enhancers that increase transcription selectively.

FIG. 1A, EV-YF1 sequence and hypothetical mechanism of action. FIG. 1B, Effects of EV-YF1 on rat BMDM transcriptomics. FIG. 1C, IRF7, IL10 were among the most differentially expressed genes. FIG. 1D, H3K27 acetylation marks on IL10 gene by ChIP-seq after CDC-EV exposure. Peaks labeled 1, 3, and 4 are unique to CDC-EV-treated rat BMDM.

Therapeutic benefits of EV-YF1 in HCM mice. Hypertrophic cardiomyopathy (HCM) (or hypertrophic obstructive cardiomyopathy (HOCM)) is a heritable illness which can lead to HFpEF. Symptoms of HCM/HOCM include, without limitation, myocyte fiber disarray, interstitial fibrosis, cardiac hypercontractility, hypertrophy, diastolic dysfunction, weight loss, premature and/or sudden death.

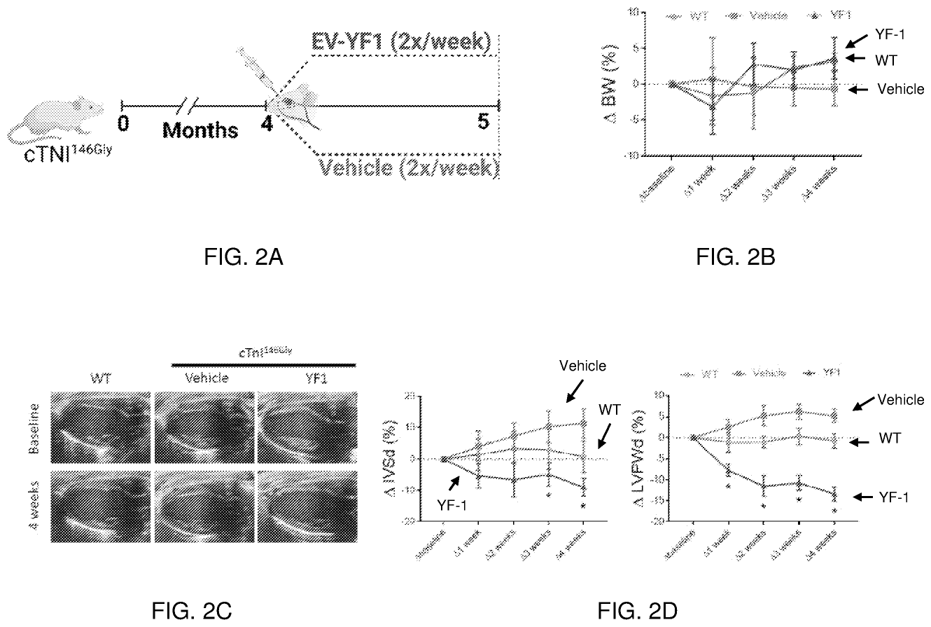

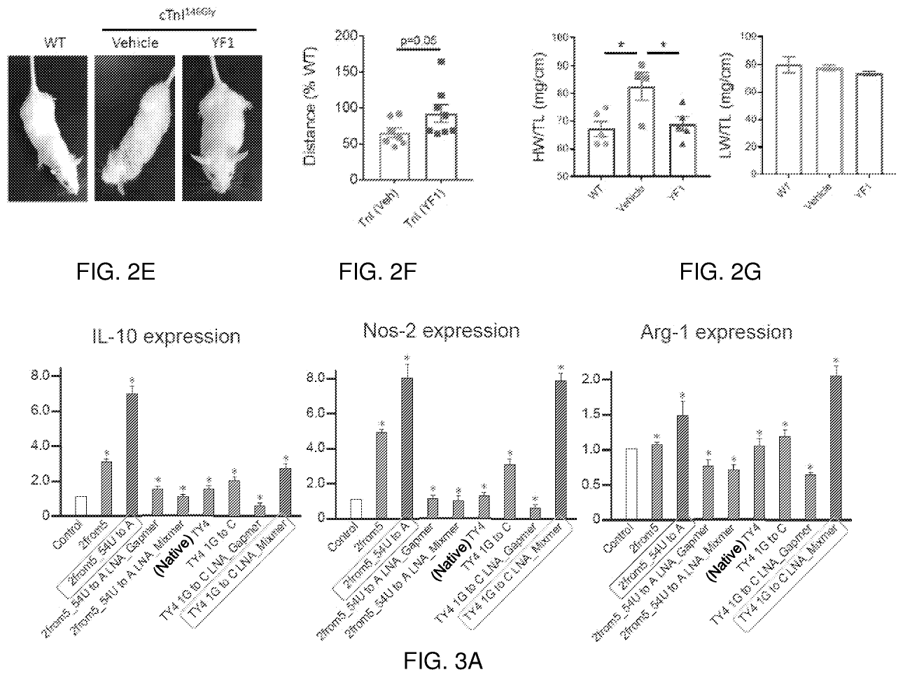

Given its efficacy in attenuating angiotensin-induced hyper-trophy, EV-YF1 (here called YF-1) was tested in transgenic cTnI^{146Gly} mice (which model a particularly lethal human HCM mutation). At 4 months of age, cTnI^{146Gly} mice received either vehicle or YF1 intravenously (via retro-orbital [r.o.] injection) twice weekly for 4 weeks (FIG. 2A). Growth was not affected (FIG. 2B), but echocardiography (FIG. 2C) revealed marked reductions in diastolic interventricular septal wall diameter (IVSd; FIG. 2D, left panel) and left ventricular posterior wall diameter (LVPWd; FIG. 2D, right panel). Overall well-being improved as evidenced by better self-grooming (FIG. 2E) and increased exercise tolerance (FIG. 2F). At endpoint, heart weight (HW)-to-tibial length (TL) ratio massively increased in the vehicle group, but decreased with YF-1; relative lung weight (LW)-to-TL was comparable in all groups (i.e., none was in overt HF with pulmonary congestion; FIG. 2G). Preliminary histology findings also showed a reduction of interstitial cardiac fibrosis with YF1. Thus, EV-YF1 is highly therapeutic in HCM mice, giving further support to HFpEF as a potential indication for EV-YF1 and derivatives thereof.

FIGS. 2A-2G: EV-YF1 in HCM mice. FIG. 2A, Protocol schematic. FIG. 2B, Body weight (BW) over time. FIG. 2C, Echo images in diastole. FIG. 2D, IVSd and LVPWd over time. FIG. 2E, Images illustrating grooming behavior. FIG. 2F, Treadmill exercise distances as percentage of distance run by WT mice. FIG. 2G, HW/TL and LW/TL at endpoint.

### Example 3

This non-limiting example shows bioactivity of EV-F1 variants, including TY4, in bone marrow-derived macrophages (MBDM).

Modified truncated mutant of EV-FY1 as lead candidate. While FIGS. 2A-2G demonstrate that EV-FY1 is itself bioactive in several disease models, it is relatively long for a synthetic RNA drug (most are 18-30 nt), and, being chemically unmodified, it is potentially unstable and immunogenic. To bypass these limitations, several EV-YF1-inspired molecules were synthesized and screened in BMDM. The candidate molecules tested are listed in the table below. LNA residues are indicated in bold.

TABLE 1

| | | SEQ ID |
|---|---|---|
| EV-YF1 variant | Sequence | NO: |
| 2from5 | CUGGUCCGAUGGUAGUGGGUUAUCAGAACUUA | 7 |
| | UUAACAUUAGUGUCACUAAAGU | |
| 2from5_54U to A | CUGGUCCGAUGGUAGUGGGUUAUCAGAACUUA | 8 |
| | UUAACAUUAGUGUCACUAAAGA | |
| 2from5_54U to A | CUGGUCCGAUGGUAGUGGGUUAUCAGAACUUA | 9 |
| LNA_Gapmer | UUAACAUUAGUGUCACUAAAGA | |
| 2from5_54U to A | CUGGUCCGAUGGUAGUGGGUUAUCAGAACUUA | 10 |
| LNA_Mixmer | UUAACAUUAGUGUCACUAAAGA | |
| TY4 (Native) | GGUCCGAUGGUAGUGGGUUAUCAG | 11 |
| TY4 1G to C | CGUCCGAUGGUAGUGGGUUAUCAG | 12 |
| TY4 1G to C | CGUCCGAUGGUAGUGGGUUAUCAG | 13 |
| LNA_Gapmer | | |
| TY4 1G to C | CGUCCGAUGGUAGUGGGUUAUCAG | 2 |
| LNA_Mixmer | | |

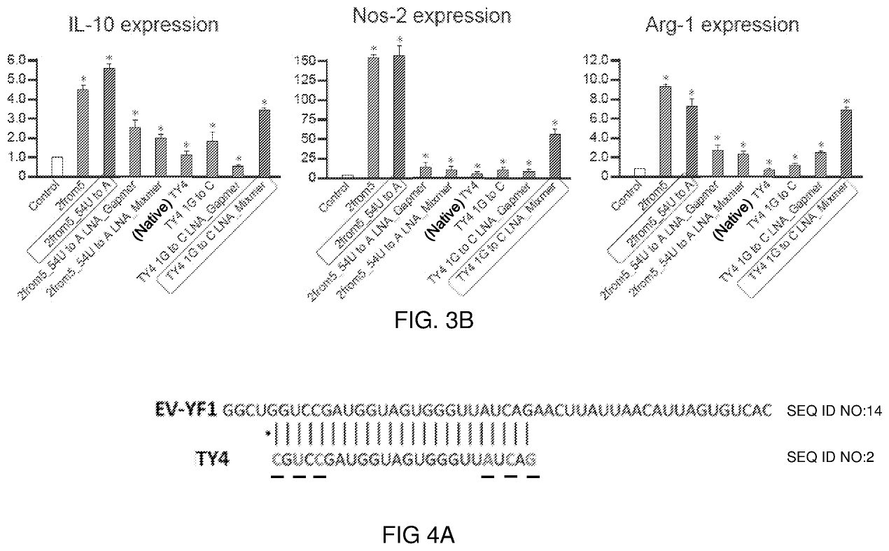

Screening of EV-YF1 variants identified two that exhibited increased bioactivity in the macrophage in vitro assay (FIGS. 3A, 3B). Transfection of “2from5_54U to A” and “TY4 1G to C LNA_Mixmer” in BMDM induced increased expression of IL-10, Nos-2, and Arg-1, recapitulating the effect of EV-YF1 on macrophages. “TY4 1G to C LNA_Mixmer” is also referred to as “TY4” herein, unless indicated otherwise. “TY4” is to be distinguished from “TY4” that is indicated to be “Native”, as in Table 1 above and FIGS. 3A and 3B. “Control” indicates BMDM treated with vehicle.

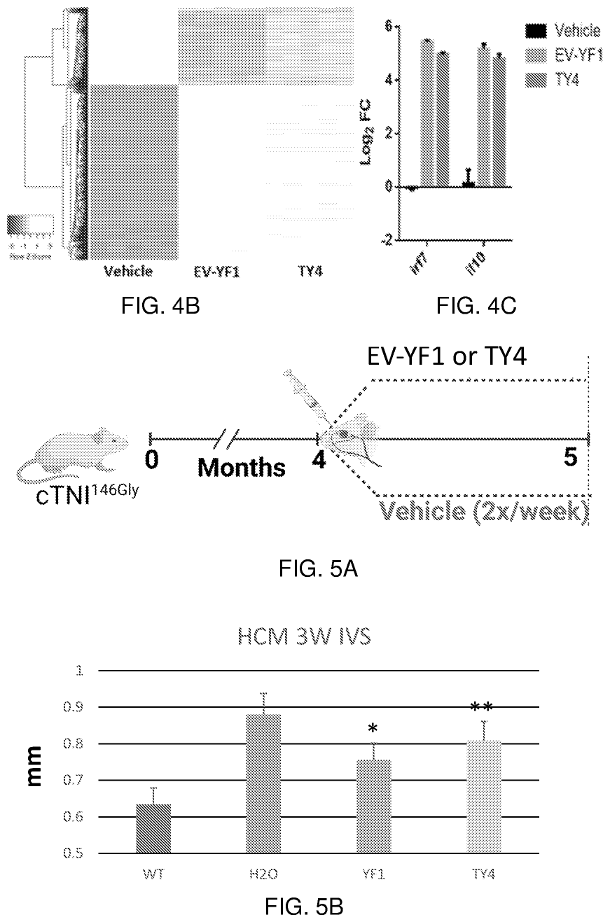

FIGS. 4A-4C further show that TY4, a 24-nt modified RNA (with locked nucleic acid substitutions) mutant derivative of EV-YF1 (FIG. 4A), affects gene expression in BMDM in a pattern like that of EV-YF1 itself (both globally [FIG. 4B] and in terms of IRF7 and IL10 [FIG. 4C]).

FIGS. 4A-4C: TY4 derived from EV-YF1. FIG. 4A, TY4 aligned with the parent EV-YF1. Underlining indicates chemically-modified nucleotides. FIG. 4B, RNA heat maps in rat BMDM. FIG. 4C, TY4 reproduces EV-YF1 effects on IRF7 and IL10.

In some embodiments, truncated and/or chemically modified variants of EV-YF1, e.g., TY4 or an isolated RNA having a sequence of any one of SEQ ID NOs:2, 11-14, induce transcriptional changes in macrophages. In some embodiments, truncated and/or chemically modified variants of EV-YF1, e.g., TY4 or an isolated RNA having a sequence of any one of SEQ ID NOs:2, 11-14, induce upregulation of IL10 and/or IRF7 in macrophages. In some embodiments, truncated and/or chemically modified variants of EV-YF1, e.g., TY4 or an isolated RNA having a sequence of any one of SEQ ID NOs: 2, 11-14, recapitulate the effect of EV-YF1 on macrophages. In some embodiments, a truncated EV-YF1 variant has the nucleotide sequence: CGUCCGAUGGUAGUGGGUUAUCAG (SEQ ID NO: 12). In some embodiments, a truncated, chemically modified EV-YF1 variant has the nucleotide sequence: CGUCCGAUGGUAGUGGGUUAUCAG (SEQ ID NO: 2), where positions 1, 3, 5, 20, 22 and 24 are locked nucleic acid (LNA).

### Example 4

This non-limiting example shows the therapeutic effect of TY4 in a model of HCM.

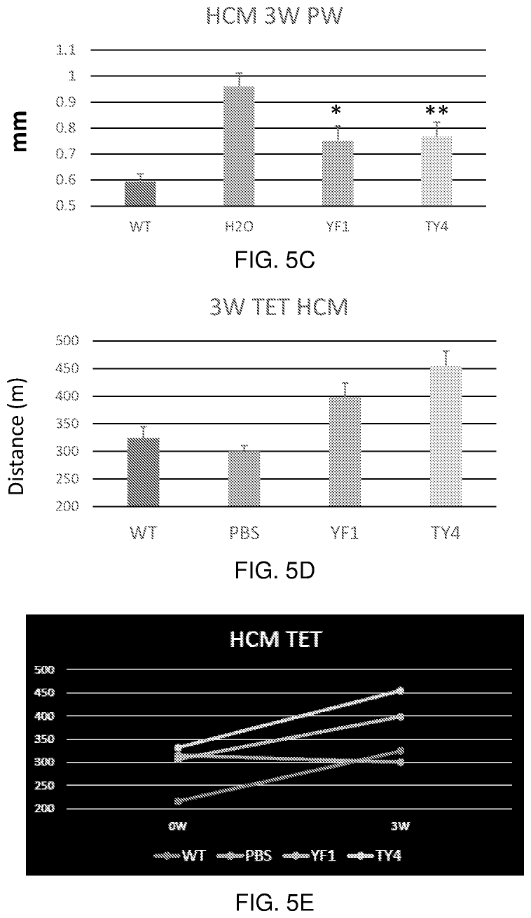

Therapeutic benefits of TY4 in HCM. TY4 recapitulated the therapeutic effect of EV-YF1 in the animal model of HCM, as described in Example 2 above. At 4 months of age, cTnI^{146Gly} mice received vehicle, EV-YF1, or TY4 intravenously (0.15 μg/g body weight) via retro-orbital [r.o.] injection) twice weekly for 4 weeks (FIG. 5A). 3 weeks after administration began, animals administered TY4 showed markedly reduced diastolic interventricular septal wall diameter (IVSd; FIG. 5B) and left ventricular posterior wall diameter (LVPWd; FIG. 5C), similar to the effect of administering EV-YF1. Thus, remodeling of the interventricular septal wall and posterior wall were attenuated by TY4 and EV-YF1 treatment. Exercise endurance also improved in TY4-treated cTnI^{146Gly} animals, which exhibited greater exercise distance over cTnI^{146Gly} animals that received saline (FIG. 5D), and exhibited a similar increase in endurance over time as wild-type animals (FIG. 5E). The therapeutic effect of TY4 on exercise endurance was greater than the effect observed in EV-YF1-treated animals (FIGS. 5D, 5E).

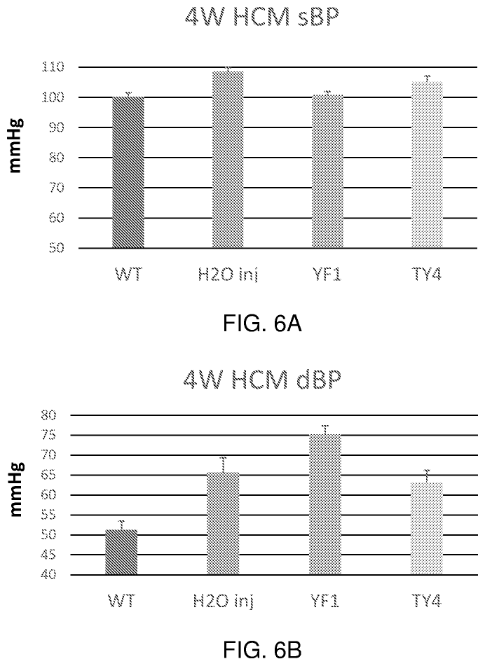

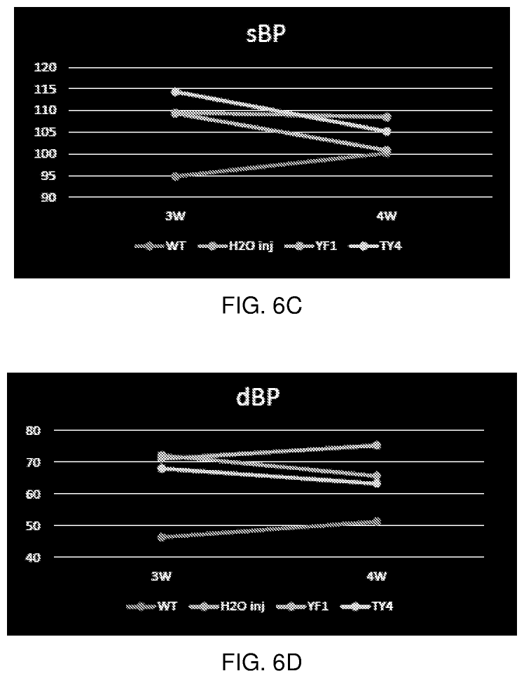

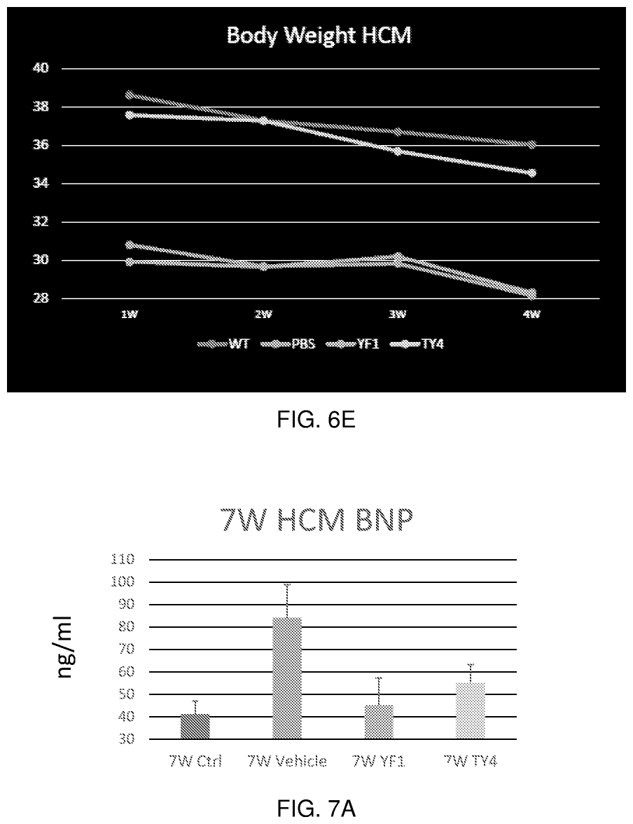

At 4 weeks, TY4 administration alleviated impaired diastolic relaxation in HCM animals (FIGS. 6A-6D). TY4-treated cTnI^{146Gly} animals had lower diastolic blood pressure than YF1-treated cTnI^{146Gly} animals (FIG. 6B). Further, at 4 weeks, body weight of TY4-treated cTnI^{146Gly} animals were comparable to wild-type control animals, whereas cTnI^{146Gly} animals administered saline had reduced body weight (FIG. 6E). EV-YF1-treated animals also had reduced body weight comparable to cTnI^{146Gly} animals that were administered saline. Therefore, TY4 exerts enhanced therapeutic bioactivity compared to EV-YF1 in a mouse model of HCM.

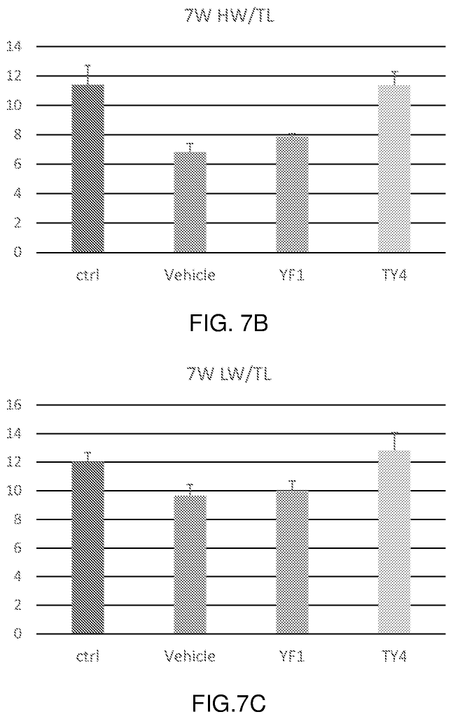

At 7 weeks, TY4-administered animals showed greatly reduced systemic inflammation, as measured by brain natriuretic peptide (BNP) levels (FIG. 7A). The reduced level was comparable to that of animals treated with EV-YF1. TY4 administration also attenuated weight loss/wasting at 7 weeks (as measured by the ratio of heart weight to tibia length, or lung weight to tibia length) (FIGS. 7B, 7C).

These results show that TY4 fully recapitulates the therapeutic effect of EV-YF1 in a model of hypertrophic cardiomyopathy.

In some embodiments, intravenously administering therapeutically effective amounts of TY4 to a subject having hypertrophic cardiomyopathy (HCM) treats the HCM. In some embodiments, intravenously and repeatedly administering therapeutically effective amounts of TY4 to a subject having hypertrophic cardiomyopathy (HCM) treats the HCM. In some embodiments, intravenously administering therapeutically effective amounts of TY4 to a subject having HCM reduces or alleviates one or more symptoms of HCM. In some embodiments, intravenously administering therapeutically effective amounts of TY4 to a subject having HCM reduces or alleviates one or more of increased IVSd, increased LVPWd, impaired exercise endurance, elevated blood pressure, systemic inflammation, and weight loss, due to the HCM.

### Example 5

This non-limiting example shows the therapeutic effect of TY4 in a model of heart failure with preserved ejection fraction (HFpEF).

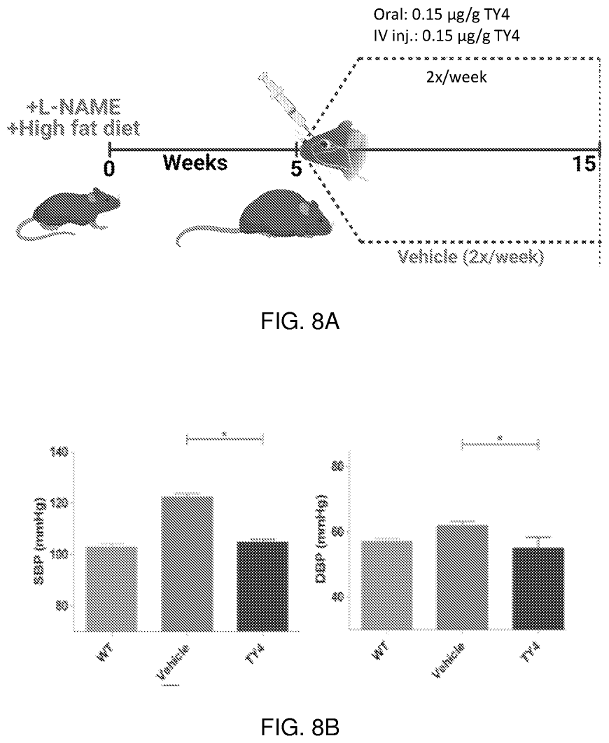

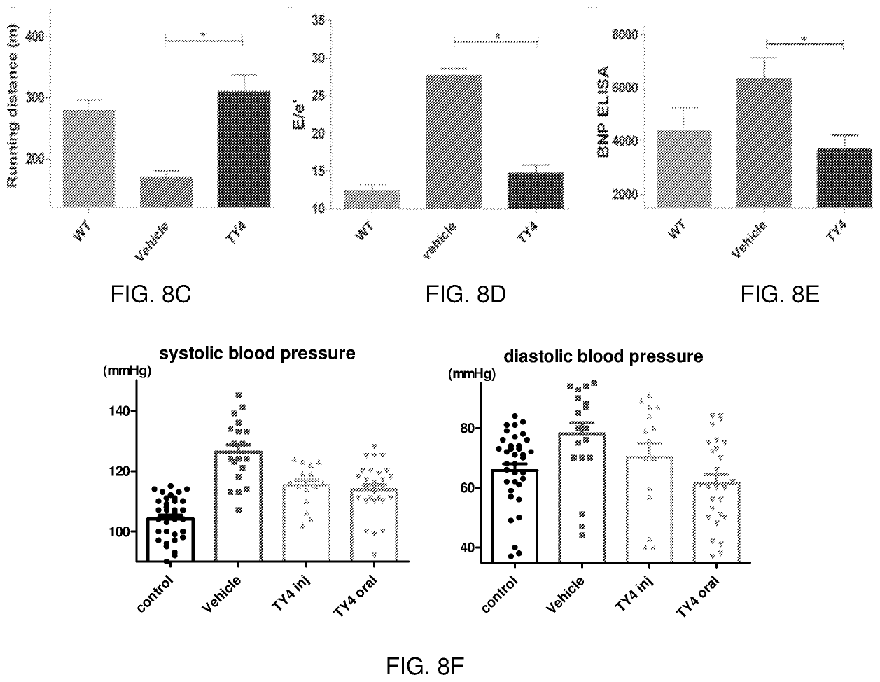

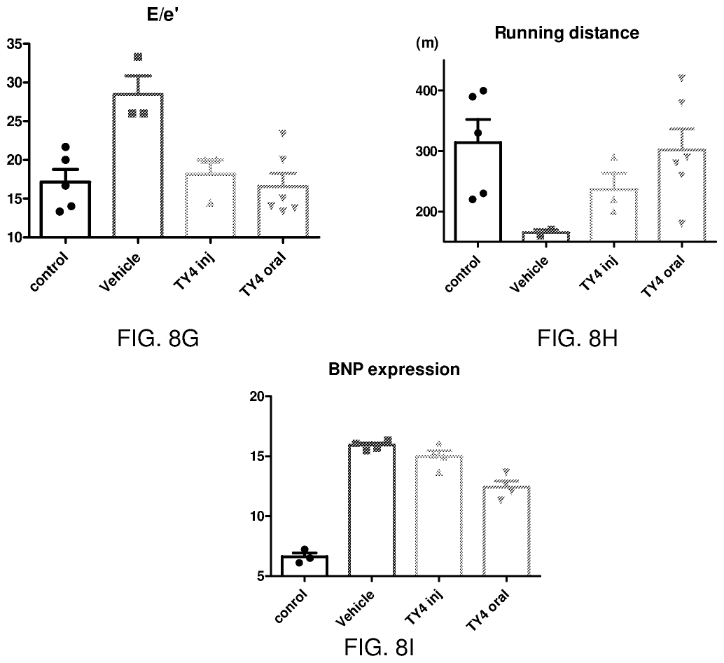

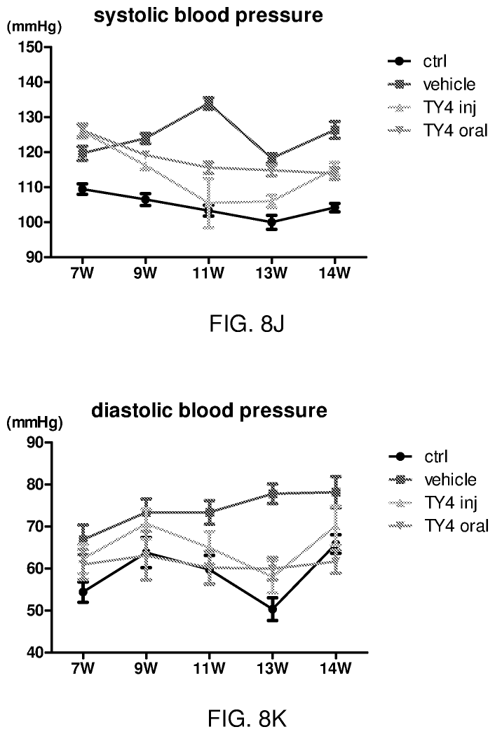

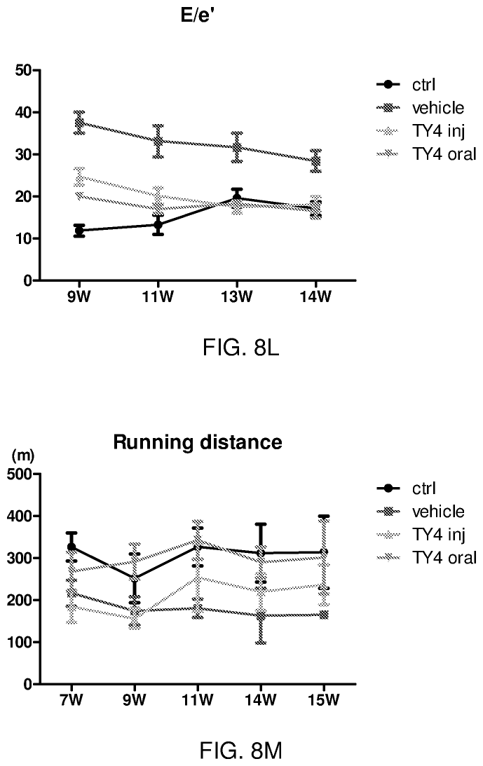

Therapeutic benefits of TY4 in HFpEF. Given the bioactivity of EV-YF1 in models of hypertrophy (Example 2) and similar therapeutic potency of TY4 in the same model (Example 4), we tested TY4 in mice with HFpEF. This “two-hit” model incorporates two comorbidities commonly associated with human HFpEF (obesity and hypertension) and reproduces nitric oxide signaling abnormalities seen in heart tissue from HFpEF patients. FIG. 8A depicts the experimental protocol. Mice were either observed without intervention (WT) or fed a high-fat diet (HFD) and L-NAME-supplemented water. Within 5 weeks, the HFD/L-NAME mice become obese and hypertensive, with diastolic dysfunction but normal EF (by baseline echos). The HFpEF mice were then randomly assigned to receive twice-weekly r.o. injection (“IV Inj.”) or oral dose (“Oral”) of vehicle or TY4. TY4 was administered at 0.15 μg/g per injection or per oral administration. For oral administration, TY4 was first combined with liposomes to form a TY4-liposome complex, which was then encapsulated in a casein-chitosan complex. Pre-infusion/pre-oral administration and at various time points later, mice underwent blood pressure measurements, treadmill testing, echocardiography, and/or blood draws for circulating biomarkers. The only differences among groups at baseline were those associated with disease (hypertension, low exercise tolerance, elevated E/e′ ratios in HFpEF vs WT).

After 10 weeks, the following differences were evident: in TY4 animals (IV injected) blood pressure was lower (FIG. 8B), exercise tolerance was higher (FIG. 8C), E/e′ ratios were lower (FIG. 8D), and brain natriuretic peptide (BNP) levels were lower (FIG. 8E) as compared to vehicle-administered HFpEF animals. These indices of health were comparable to WT levels (non-HFpEF animals). This effect was not due to dietary aversion: the TY4 mice remained obese. The findings reveal striking disease-modifying bioactivity of TY4, especially remarkable given the refractory nature of HFpEF.

FIGS. 8A-8C: TY4 administered intravenously reverses disease progression in HFpEF mice. FIG. 8A: schematic study design. At study endpoint animals which received TY4 intravenously had lower systolic (SBP) and diastolic blood pressure (DBP) (FIG. 8B), improved exercise tolerance (FIG. 8C) and diastolic function (E/e′, FIG. 8D), and reduced levels of a serum biomarker of heart failure (BNP; FIG. 8E).

Similar therapeutic effects, and in some instances superior therapeutic results, were observed in animals treated orally with TY4. Oral administration of TY4 reduced blood pressure (FIG. 8F), reduced E/e′ ratios (FIG. 8G), increased exercise tolerance (FIG. 8H), and reduced BNP levels (FIG. 8I) as compared to vehicle-administered HFpEF animals. Blood pressure, exercise tolerance and E/e′ ratios were comparable to WT levels (non-HFpEF animals) in animals treated orally with TY4.

FIGS. 8F-8I: TY4 administered orally reverses disease progression in HFpEF mice. The experimental protocol is shown in FIG. 8A. At study endpoint, animals which received TY4 orally had lower systolic (SBP) and diastolic blood pressure (DBP) (FIG. 8F), improved diastolic function (E/e′, FIG. 8G) and exercise tolerance (FIG. 8H), and reduced levels of a serum biomarker of heart failure (BNP; FIG. 8I).

The therapeutic effect of TY4 was observed consistently over the course of treatment in both intravenously and orally administered animals. Reduced systolic blood pressure was observed as soon as Week 9 (FIG. 8J), while reduced diastolic pressure was observed from Week 7 onwards (FIG. 8K). Improved E/e′ ratios were observed from Week 9 and at least through Week 14 (FIG. 8L). Improved exercise tolerance was seen more consistently in orally treated animals, and all TY4-treated animals showed higher exercise tolerance by at least Week 11 (FIG. 8M).

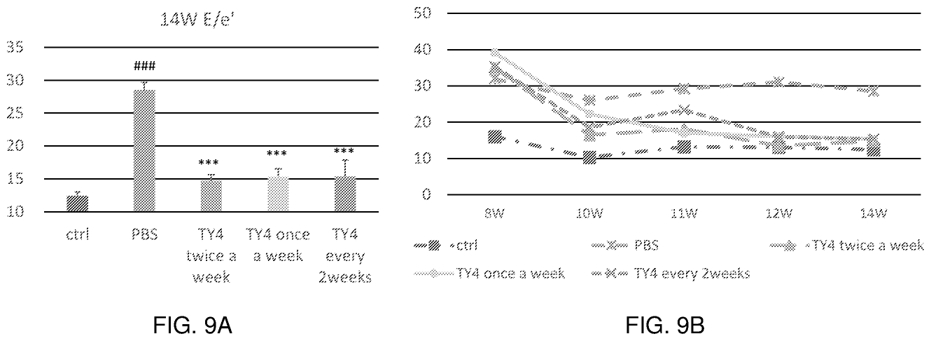

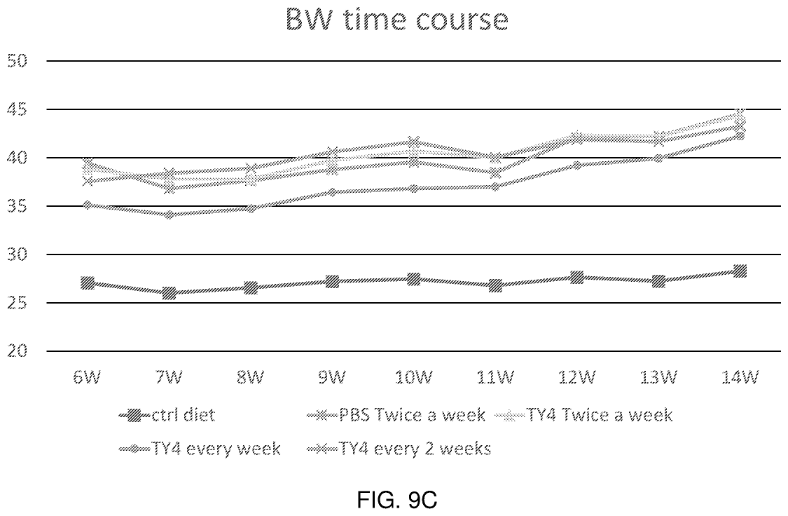

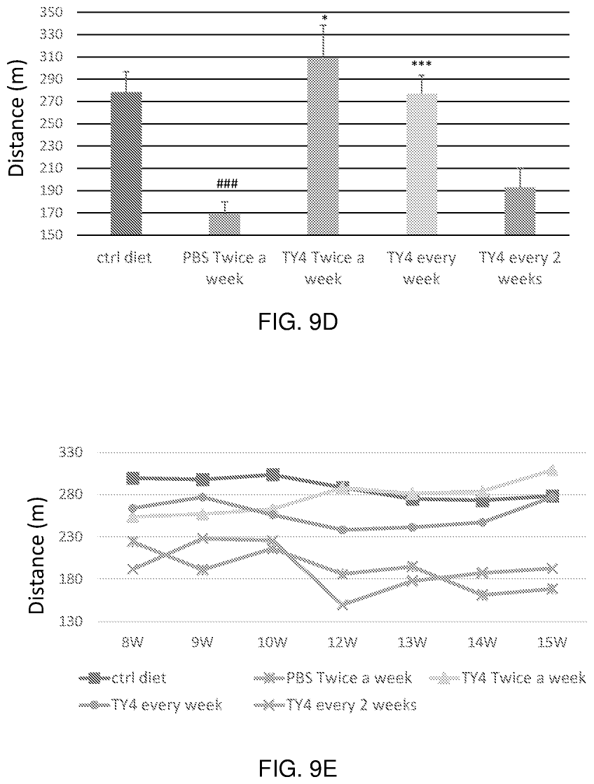

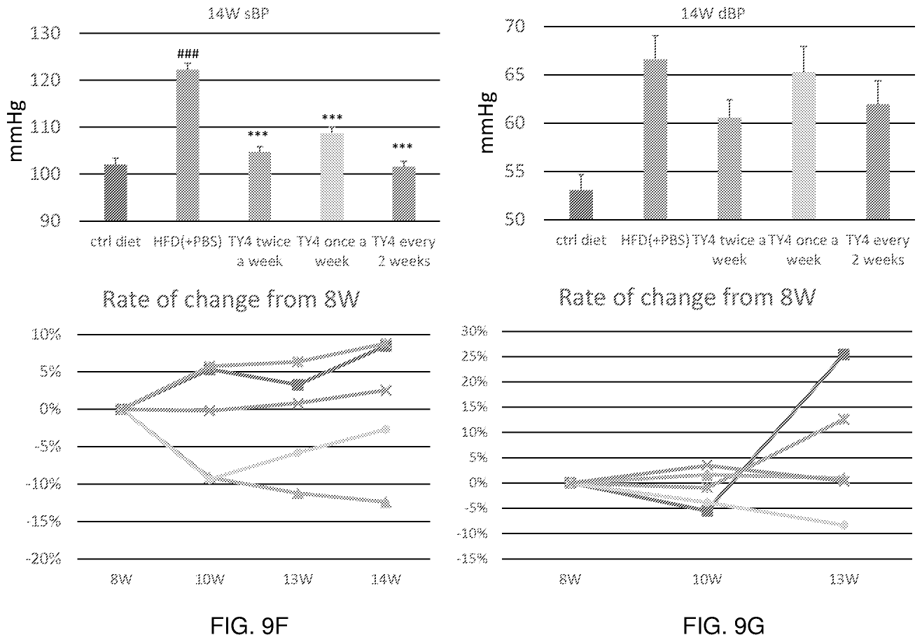

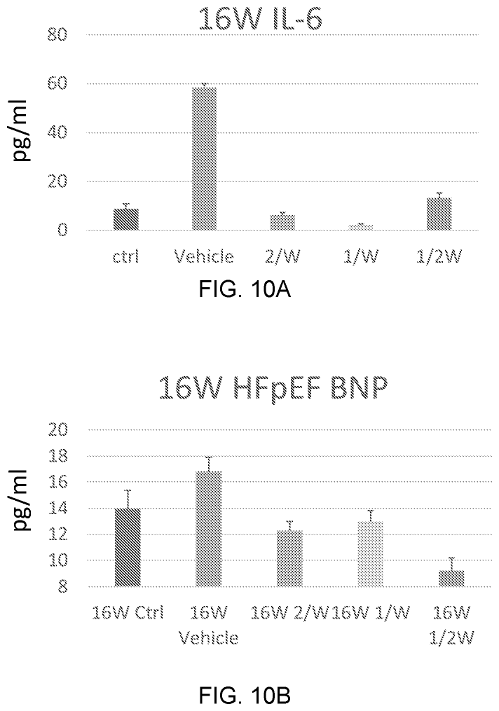

To test the effect of dosing frequency on the therapeutic effect of TY4 in HFpEF, an experimental protocol as in FIG. 8A was performed except the HFD/L-NAME animals were dosed intravenously at a frequency of twice a week, once a week or once every two weeks. At 14 weeks, animals treated with TY4 intravenously reduced E/e′ ratios were observed at all frequencies, indicating attenuation of diastolic dysfunction (FIGS. 9A, 9B). As before, the therapeutic effect was not due to dietary aversion as weight gain was not altered at all three dosing frequencies (FIG. 9C). Exercise endurance improved in a dosing frequency-dependent manner, as consistent improvement was seen at an administration frequency of once or twice a week, while there was little improvement when administered once every two weeks (FIGS. 9D, 9E). TY4 attenuated hypertension (FIGS. 9F, 9G). In particular, at administration frequencies of twice a week and once a week, systolic blood pressure declined in the period after 8 weeks, while at administration of TY4 once every two weeks, systolic pressure remained the same over the same period (FIG. 9F, lower panel). TY4 attenuated systemic inflammation at all administration frequencies, as measured by IL-6 and BNP levels at 16 weeks (FIGS. 10A, 10B).

In some embodiments, intravenously or orally administering therapeutically effective amounts of TY4 to a subject having HFpEF treats the HFpEF (or one or more symptoms thereof). In some embodiments, repeatedly administering (intravenously or orally) therapeutically effective amounts of TY4 to a subject having HFpEF treats the HFpEF (or one or more symptoms thereof). In some embodiments, intravenously or orally administering therapeutically effective amounts of TY4 to a subject having HFpEF reduces or alleviates one or more symptoms of HFpEF. In some embodiments, intravenously or orally administering therapeutically effective amounts of TY4 to a subject having HFpEF reduces or alleviates one or more of impaired exercise endurance, elevated blood pressure, elevated E/e′ ratio, and systemic inflammation, due to the HFpEF.

### Example 6

This non-limiting example shows determining biodistribution of CDC-EVs by measuring human-specific Y-RNA sequences.

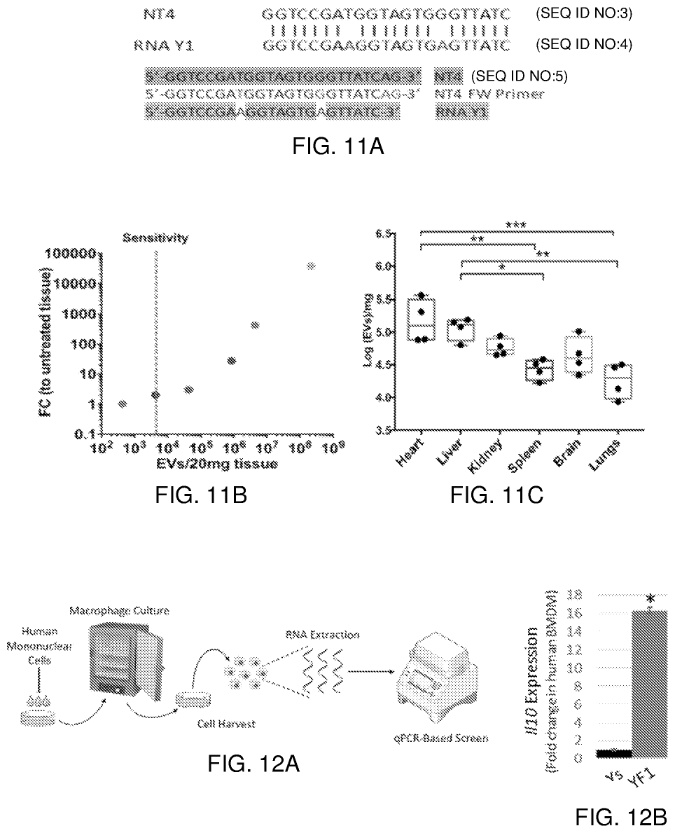

To assess biodistribution, quantitative PCR (qPCR) primers were designed to amplify part of the sequence of EV-YF1 because of its abundance in CDC-EVs. FIG. 11A shows that the chosen region (here denoted as NT4) does not have high homology with the closest mouse sequence (RNA Y1). Human CDC-EVs spiked into homogenized mouse liver tissue could be resolved at 10⁴ EVs/20 mg (FIG. 11B), enabling quantification of biodistribution one hour after r.o. injection (FIG. 11C).

FIGS. 11A-11C: Biodistribution of CDc-EVs. FIG. 11A: human and mouse sequences and forward PCR primer. FIG. 11B: limit of resolution at 10³-10⁴ EVs/20 mg tissue. FIG. 11C: CDc-EVs per g of tissue 1 hr after r.o. injection of 2×10⁹ CDC-EVs.

In some embodiments, biodistribution of CDC-EVs and/or cargo contents thereof can be assessed in a non-human animal by detecting human-specific Y-RNA sequences known to be in CDC-EVs, e.g., human CDC-EVs, in tissues after administration of the CDC-EVs to the animal.

### Example 7

This non-limiting example shows an experimental design for testing bioactivity of candidate therapeutic molecules, e.g., TY4 and/or variants thereof, in vitro and in vivo.

A candidate therapeutic is tested for selected changes in gene expression in human macrophages (HM). Those with transcriptomic patterns (mimicking at least partly those induced by known salutary variants) is tested in vivo. Those candidates that proceed to in vivo testing are characterized in two complementary, well-established models of HFpEF.

Macrophage screen. The overall methods are depicted in FIG. 12A. Human bone marrow mononuclear cells are obtained and are differentiated in IMDM (supplemented with 10% FBS, 1% penicillin/streptomycin, and human M-CSF [R&D Systems]) to create HM over 6-7 days, analogous to what was done with rat macrophages in Examples 2 and 3. The data in FIG. 12B verify that HM can be cultured and that they respond similarly to EV-YF1 in terms of IL10 mRNA upregulation. TY4 and derivatives are synthesized, and mixed with 5 μl of small RNA transfection reagent (DharmaFECT, GE) to a final volume of 100 μl in serum-free media. Following agitation and incubation (to form liposomal-RNA complexes), the preparation is added to the HM culture media to a final concentration of 80 nM. The same process is repeated with each of the new chemical variants of TY4, and an inert scrambled sequence. RNA is extracted 24 hours later and subjected to analysis by quantitative polymerase chain reaction (qPCR) to determine transcript levels of the anti-inflammatory cytokine IL10, inducible nitric oxide synthase (NOS2), and HPRT1, a housekeeping enzyme used as an endogenous control. IL10 can serve as a positive potency marker, and inducible nitric oxide synthase (NOS2) can serve as a negative potency marker. To pass this screen and proceed to in vivo testing, any given variants increases IL10 mRNA levels at least 3-fold, while not increasing NOS2 mRNA levels, both normalized by HPRT1 mRNA levels and compared to the mRNA levels in HM transduced with scrambled sequence.

Rodent models. The mouse model of HFpEF (Model 1) is one in which preliminary data show disease-modifying bioactivity with TY4 (FIGS. 8A-8M). Wild-type mice is placed on a high-fat diet and water supplemented with L-NAME (or normal diet and water as healthy controls) for a total of 15 weeks. At 5 weeks (when obesity and diastolic dysfunction are already evident), animals are randomly assigned to receive a candidate molecule or vehicle control. Animals receive bi-weekly infusions of ncRNA or placebo, at a starting dose of 0.15 μg/g of body weight by r.o. injection or orally. The dose and/or dosing interval is adjusted as part of systematic dose-finding studies. Dose escalation studies can be carried to achieve beneficial results. Once a dosing regimen is established, blood pressure, echocardiography, exercise endurance, and serum BNP (a biomarker of heart function) is assessed every 2 weeks. At the study endpoint (15 weeks of high-fat diet+L-NAME, and 10 weeks after initiating interventions), final phenotyping is performed, and the hearts are removed under anesthesia for histology. Myocardial tissue is sectioned and stained for fibrosis (Masson's trichrome), inflammation (hematoxylin and eosin, plus immunohistochemistry for CD45 [for all leukocytes], and CD68 [for macrophages]).

The two-hit mouse model, which has negligible mortality over months of established disease, and the Dahl salt-sensitive (DS) rat, which has comorbidities of hypertension, insulin resistance, and hyperlipidemia, is used. Unlike the two-hit model, DS rats on a high-salt diet develop severe and rapidly-progressive disease with high mortality, enabling the use of survival as an endpoint. DS rats are maintained on a 12-hour light/dark cycle from 6 AM to 6 PM and have unrestricted access to food and water. Seven-week-old DS rats are assigned to low-salt (0.3% salt; controls) or high-salt diet (8% salt). After 6-7 weeks, animals are phenotyped by echocardiography to ensure and quantify diastolic dysfunction (E/A and E/e′ ratios), and to look for systolic dysfunction or LV dilation (˜10% of our DS rats fed high-salt diet develop HFrEF, which can be excluded). After baseline phenotyping, rats are treated with placebo or each ncRNA, using the dosing regimens described above (which will be tailored as needed). After the intervention, animals are followed until death or 4 weeks, whichever comes earlier (mortality is ˜50% at that time in placebo rats at that point). Survivors are thoroughly phenotyped using echocardiography again at 4 weeks, after which rats are euthanized and hearts removed for histology.

### Example 8

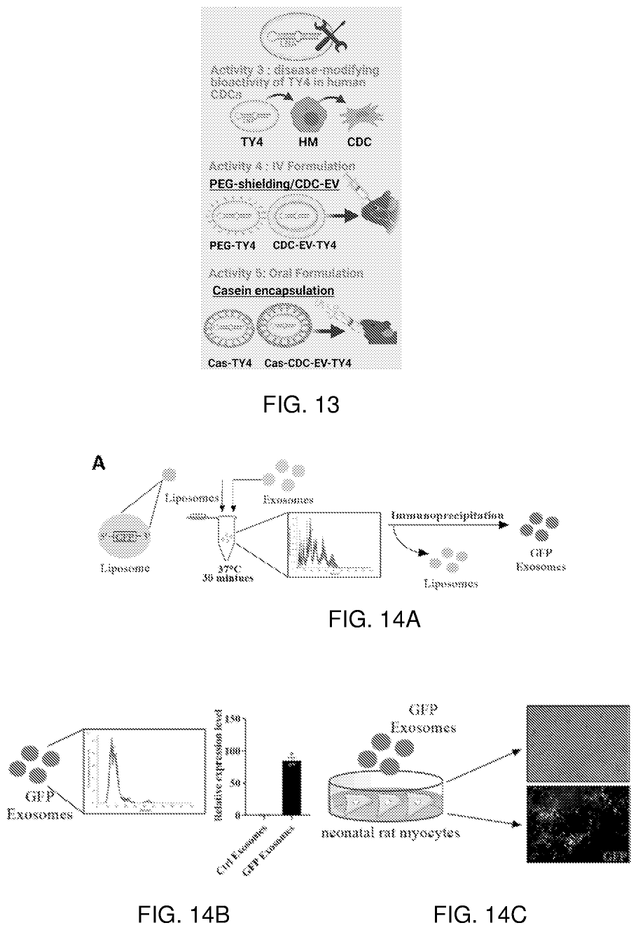

This non-limiting example shows an experimental design to assess injury-modifying bioactivity in an in vitro assay using human allogeneic CDCs (a clinically-relevant progenitor cell population) in coculture with macrophages (FIG. 13).

Human CDCs, from each of two banked lines qualified for clinical use, is subjected to serum-free conditions and oxidative stress (H₂O₂, 75 μmol/L for 20 min) to induce apoptosis. Afterwards, the stressed CDCs are cocultured with human macrophages (HM), which had been exposed either to a therapeutic compound of the present disclosure (e.g., TY4 or derivative thereof), or to the transfection reagent alone, for 24 hours. After 6 hours of coculture, CDC apoptosis is quantified by TUNEL assay (TUNEL+ CD105+ cells/total CD105+ cells [%]).

To test the direct effect of a therapeutic compound of the present disclosure (e.g., TY4 or derivative thereof), the compound (or transfection reagent alone) are exposed directly to CDCs and differences in apoptosis are quantified, without intervening HM.

In some embodiments, treating human macrophages (HM) with TY4 and/or a derivative thereof induces or promotes the ability of HM to reduce of suppress apoptosis in stressed CDCs. In some embodiments, treating stressed CDCs with TY4 and/or a derivative thereof reduces or suppresses apoptosis.

### Example 9

This non-limiting example shows a study design to test different formulations for in vivo delivery of TY4 and/or derivatives thereof (FIG. 13).

PEG shielding. PEG-cationic lipid complexes (PCLC) are formed using a mixture of 2 kDa polyethylene glycol (PEG2000; 30% v/v) and Dharmafect. Complexes are formed using five freeze/thaw cycles (liquid nitrogen/60° C.) as adapted from previous preparations. A single freeze-thaw cycle involves freezing the mixture for 5 min at −190° C. (liquid nitrogen) followed by thawing for 5 at 60° C. Complexes of TY4 (and/or a derivative thereof) with PCLC are made by admixing appropriate concentrations of TY4 (and/or a derivative thereof) with 5 μl of PCLC to a final volume of 1001. The preparation is incubated at room temperature for 5 min with agitation.

In some embodiments, TY4 (and/or a derivative thereof) is formulated as a complex with PCLC. In some embodiments, a pharmaceutical composition of TY4 (and/or a derivative thereof) includes TY4 (and/or a derivative thereof) and PCLC.

### Example 10

This non-limiting example shows cellular uptake of chemically modified RNA packaged into exosomes.

LNA-modified mRNA encoding GFP was loaded into CDc-EVs. First, standard formulation liposomes containing the modified mRNA were created. The modified mRNA-liposome formulation was admixed with CDc-EVs (isolated from serum-free media conditioned by human CDCs in culture) at 37° C. in a shaker for 30 min. Liposome-exosome complex size distributions, measured by dynamic light scattering (NanoSight NS300), revealed multiple peaks (FIG. 14A), indicative of heterogeneous populations. To select out exosomes, immunoprecipitation with anti-CD9, anti-CD63, and anti-CD81 antibodies (all of which target exosome-specific surface proteins) was used. As shown in FIG. 14B, immunoprecipitants showed a single peak (diameter ˜120 nm), characteristic of exosomes. By qPCR, GFP mRNA levels were markedly greater than in control exosomes. Addition of immunoprecipitated exosomes to neonatal rat cardiomyocytes in culture induced GFP expression, as observed by epifluorescence 24 hrs later (FIG. 14C), verifying the ability of functional transgene expression from modified GFP mRNA loaded into the exosomes.

In some embodiments, a therapeutic compound of the present disclosure, e.g., TY4 and/or derivatives thereof, is loaded into exosomes (FIG. 13). In some embodiments, the therapeutic compound of the present disclosure (e.g., TY4 or a derivative thereof) is loaded into CDC-EV and immunoprecipitation is used to select out CDC-EV.

### Example 11

This non-limiting example shows a study design to test different routes of administration for in vivo delivery of TY4 and/or derivatives thereof (FIG. 13).

In vivo studies. Three formulations (default, PEG, exosomes), including those shown in Examples 9 and 10, are compared for disease-modifying bioactivity in the mouse HFpEF model. Phenotyping and terminal analyses is performed as shown in Example 7. Likewise, the choice of initial dose, and the permutations of dose and dosing interval, is made as shown in Example 7. Once a preferred dosing regimen and formulation are chosen, biodistribution will be assessed using the approach in Example 6.

Criteria to be weighed in choosing the preferred formulation can include the magnitude of therapeutic benefits on heart function and serum biomarkers, exercise tolerance, and histological benefits (on fibrosis and inflammation).

In some embodiments, liposomes of TY4 (and/or a derivative thereof) mixed with a conventional transfection reagent provides in vivo delivery of therapeutically effective amounts of TY4 (and/or a derivative thereof) to a subject by intravenous or oral administration. In some embodiments, PEG shielding of TY4 (and/or a derivative thereof) improves uptake of the liposomes and/or payload delivery and/or pharmacokinetics of TY4 (and/or a derivative thereof) after intravenous delivery. In some embodiments, PEG shielding of TY4 (and/or a derivative thereof) promotes oral uptake of TY4 (and/or a sderivative thereof). In some embodiments, PEG shielding of TY4 (and/or a derivative thereof) improves uptake of the liposomes and/or payload delivery and/or pharmacokinetics of TY4 (and/or a derivative thereof) after intravenous delivery. In some embodiments, PEG shielding of TY4 (and/or a derivative thereof) promotes oral uptake of TY4 (and/or a derivative thereof).

### Example 12

This non-limiting example shows formulation of CDC-EV with casein for oral administrations.

Unaltered CDc-EVs can be taken up when given orally. Casein, the dominant protein in breast milk, can enhance the uptake and bioactivity of ingested CDc-EVs, altering gene expression in blood cells and enhancing muscle function in mdx mice.

Quantifying oral uptake. Liposomes or EVs are counted as described above in Example 11 (NanoSight). A starting “dose” of 10⁷ particles is chosen. The therapeutic compound of the present disclosure (e.g., TY4 and/or a derivative thereof) is administered as is, or mixed with 8% casein solution in phosphate-buffered saline (PBS). Each therapeutic compound formulation, or the mixture of casein solution and each therapeutic compound formulation, is fed to HFpEF mice by oral gavage after 18 hours of only-food fasting, and is compared to feeding PBS alone or 8% casein solution alone after 18 hours of only-food fasting. One hour after oral administration of each test article, blood is collected from the inferior vena cava for RNA extraction. Uptake into the blood is quantified by measuring therapeutic compound levels in whole blood by qPCR, using RNA isolation methods. Following the approach in Example 6, measured PCR cycles is compared against standards created by spiking known levels of TY4 into mouse blood. TY4 is derived from a human-specific sequence, so background levels in mice are below the limit of reliable PCR detection. Selection for further characterization is based upon measured levels of TY4 in blood. A formulation (whether with or without casein) can be considered to provide oral delivery if TY4 is detected by 2 amplification cycles [Ct] of control or earlier by qPCR. In some embodiments, a formulation provides detection at >3 Ct lower than the nearest competitor by qPCR. A formulation may be further tested for in vivo bioactivity.

In vivo bioactivity. To assess disease-modifying bioactivity, the mouse HFpEF model is used. Starting 5 weeks after the initiation of high-fat diet+L-NAME, HFpEF mice will be fed vehicle (PBS), and each of the formulations advanced (with or without casein), every other day. Phenotyping and terminal analyses follows those delineated in Example 7. Once a preferred dosing regimen and formulation are chosen, biodistribution is assessed using the approach in Example 6.

In some embodiments, TY4 (and/or a derivative thereof) (in liposomes or CDC-EV) is formulated with casein. In some embodiments, a pharmaceutical composition of TY4 (and/or a derivative thereof) includes TY4 (and/or a derivative thereof) in liposomes or CDC-EV, and casein. In some embodiments, a pharmaceutical composition of TY4 (and/or a derivative thereof) includes TY4 (and/or a derivative thereof) in liposomes or CDC-EV, and 8% casein. In some embodiments, a formulation of TY4 (and/or a derivative thereof) (in liposomes or CDC-EV) and casein, e.g., 8% casein, promotes oral uptake of TY4 (and/or a derivative thereof).

### Example 13

This non-limiting example shows the therapeutic effect of TY4 in a model of Duchenne muscular dystrophy.

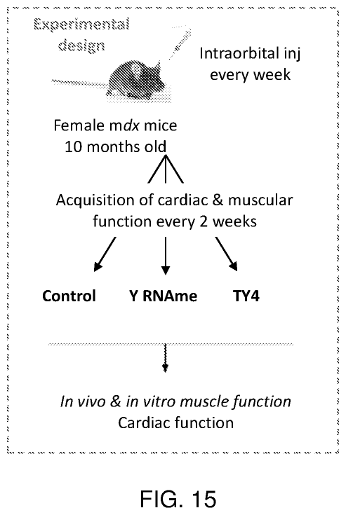

10-month old female mdx mice were administered methylated EV-YF1 (Y RNAme), TY4, or vehicle control every week (FIG. 15). Cardiac and muscle function was measured every 2 weeks.

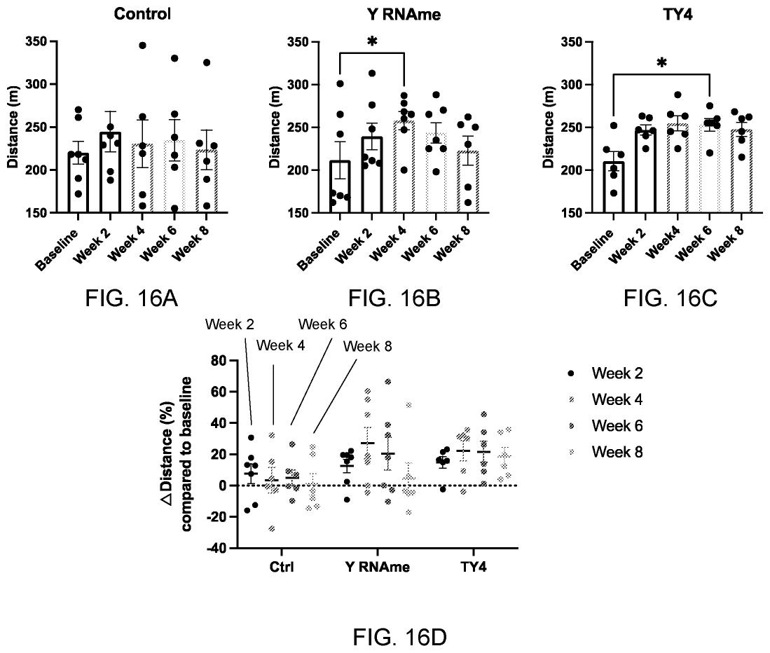

Exercise tolerance increased in mdx animals treated with EV-YF1 and TY4 compared to vehicle control (FIGS. 16A-16D). While the increased exercise tolerance in EV-YF1-treated animals was transient, peaking at around Weeks 2 and 4 (FIGS. 16B and 16D), animals treated with TY4 showed a sustained increase in exercise tolerance from Week 2 through Week 8 (FIGS. 16C and 16D).

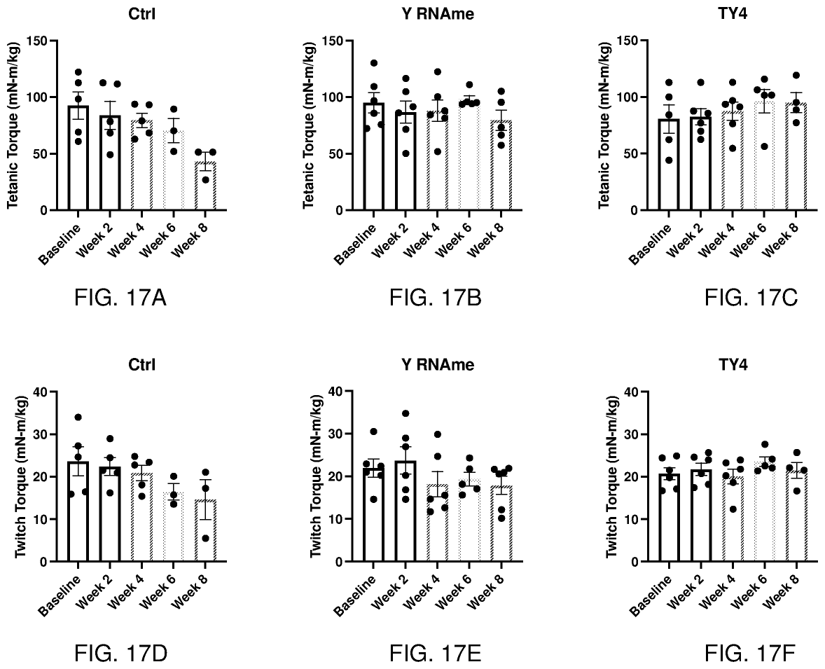

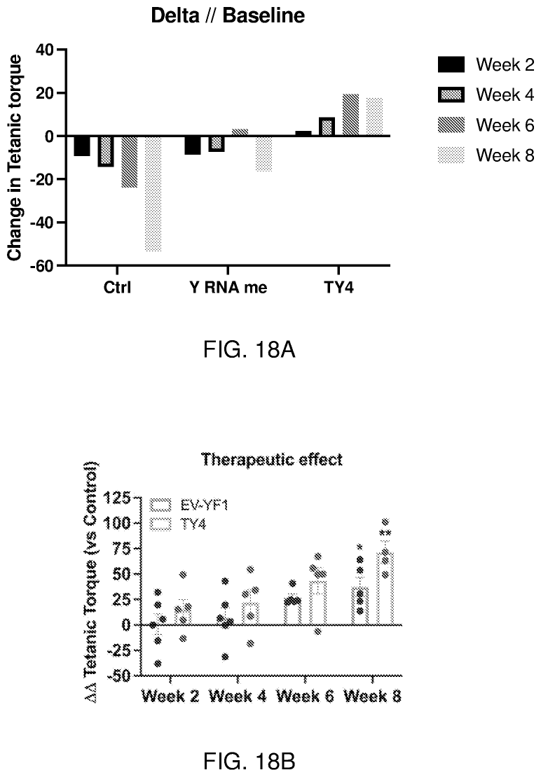

Muscle function as measured by tetanic torque and twitch torque improved in skeletal muscle of mdx animals treated with EV-YF1 and TY4 compared to vehicle control (FIGS. 17A-17F, 18A). In control animals, muscle function declined steadily over the course of the experiment (FIGS. 17A, 17B, 18A). In contrast, EV-YF1 and TY4 treatment prevented the deterioration of muscle function in mdx animals (FIGS. 17B, 17C, 17E, 17F, 18A). While tetanic torque remained relatively constant over time in EV-YF1-treated animals (FIGS. 17B, 18A), those treated with TY4 showed increased tetanic torque over time (FIGS. 17C, 18A). Thus, TY4 showed greater therapeutic effect over EV-YF1 with respect to improvement in muscle function in mdx animals (FIGS. 18A, 18B; *p<0.05, **p<0.01).

In some embodiments, administration of TY4 to a subject having muscular dystrophy (e.g., Duchenne muscular dystrophy) treats the muscular dystrophy, or one or more symptoms thereof. In some embodiments, administration of TY4 to a subject having muscular dystrophy (e.g., Duchenne muscular dystrophy) improves exercise tolerance. In some embodiments, administration of TY4 to a subject having muscular dystrophy (e.g., Duchenne muscular dystrophy) improves exercise tolerance for a sustained period. In some embodiments, administration of TY4 to a subject having muscular dystrophy (e.g., Duchenne muscular dystrophy) improves exercise tolerance for the duration of administration. In some embodiments, administration of TY4 to a subject having muscular dystrophy (e.g., Duchenne muscular dystrophy) improves skeletal muscle function. In some embodiments, administration of TY4 to a subject having muscular dystrophy (e.g., Duchenne muscular dystrophy) prevents deterioration of skeletal muscle function. In some embodiments, the therapeutic effect of TY4 administered to a subject having muscular dystrophy (e.g., Duchenne muscular dystrophy) is greater in duration and/or effect than the therapeutic effect of EV-YF1. In some embodiments, the therapeutic effect of TY4 administered to a subject having muscular dystrophy (e.g., Duchenne muscular dystrophy) is more sustained than the therapeutic effect of EV-YF1.

### Example 14

The following materials and methods were used for Examples 15-22.

### Experimental Animals

All studies ware performed at Cedars-Sinai Medical Center in accordance with the Institutional Animal Care and Use Committee guidelines.

Mouse Two-Hit Model of Heart Failure with Preserved Ejection Fraction.

Eight to ten-week-old male C57BL/6 mice were obtained from Charles River laboratories. Mice were housed under controlled with a 12:12-h light-dark cycle and had unrestricted access to food (2916, Teklad for Control groups and D12492, Research Diet for High Fat Diet groups) and water. L-NAME (Nω-Nitro-L-arginine methyl ester hydrochloride, N5751, Millipore sigma 0.5 g/l) was added in drinking water after adjusting the pH to 7.4.

Blood pressure analysis: Systolic and diastolic blood pressure were measured noninvasively in conscious mice using BP-2000 (Visitech System). Mice were placed in individual holders under 37-degree temperature. Blood pressure was recorded at least 20 times per session.

Echocardiography: Cardiac function and morphology were assessed under general anesthesia by transthoracic echocardiography using Vevo 3100 (VisualSonics). Apical four-chamber views were performed for diastolic function measurements using pulsed-wave and tissue Doppler imaging at the level of mitral valve. During echocardiography, body temperature of mice was controlled and isoflurane was reduced to under 1.0% and adjusted to maintain a heart rate in the range of 420-470 bpm.

Rat Ischemia/Reperfusion Model: I/R model, Intra-ventricular and retro-orbital injection. All rats were housed in a pathogen-free facility (cage bedding: Sani-Chips, PJ Murphy) with a 14 hours/10 hours light/dark cycle with food (PicoLab Rodent Diet 20 [no. 5053], Lab Diet) and water provided ad libitum. In vivo, experimental protocols were performed on 7 to 10-week-old female Wistar-Kyoto (Charles River Labs, Wilmington, MA). To induce ischemic injury, rats were provided general anesthesia and then a thoracotomy was performed at the fourth intercostal space to expose the heart and left anterior descending (LAD) coronary artery. A 7-0 silk suture was then used to ligate the left anterior descending coronary artery, which was subsequently removed after 45 min to allow for reperfusion for 20 min. Vehicle (PBS only), TY4 (0.15 μg/g), TY4 scramble (0.15 μg/g) formulated in with DharmaFECT transfection reagent (Horizon Discovery) were injected into the retro-bulbar space.

Rat infarct size measurement: Two days following I/R injury, 10% KCL was injected into the LV to arrest hearts in diastole. Then, hearts were harvested, washed in PBS, and then cut into 1-mm sections from apex to base, above the infarct zone. Sections were incubated with 1% solution 2,3,5-triphenyl-2H-tetrazolium chloride (TTC, Sigma-Aldrich)) for 30 minutes at 37° C. in the dark and washed with PBS. Then, sections were imaged and weighed. The infarcted zones (white) were delineated from viable tissue (red) and analyzed (ImageJ software). Infarct mass was calculated in the tissue sections according to the following formula: (infarct area/tot area)/weight (mg).

Cardiac troponin I ELISA: Blood was collected from animals at 24 hours (from the tail vein) or at the study endpoint (from the heart) in EDTA tubes. After being left undisturbed at 4° C. for 30 minutes, plasma was obtained after 15-minute centrifugation at 4000 rpm. Cardiac troponin I was quantified using the RAT cardiac troponin-I elisa kit (Life Diagnostics) according to the manufacturer's protocol.

Porcine Ischemia/Reperfusion Model: Myocardial infarction was induced in female adult Yucatan mini-pigs. Age-matched animals of similar size (30-35 kg) were enrolled. A standard balloon angioplasty catheter (TREK) was advanced distal to the first diagonal branch at the proximal third of the left anterior descending (LAD) artery. The balloon was inflated for 90 min, followed by 48 hours of reperfusion. Thirty minutes post reperfusion animals received either 0.15 μg/g of TY4, Scramble, or vehicle (saline) control. Cardiac MRI was performed at 48 hours post reperfusion. Infarct size was determined using Gentian violet, Thioflavin T, and triphenyl tetrazolium chloride (TTC) staining. This study was performed on a protocol approved by the institutional animal care and use committee at Cedars-Sinai Medical Center.

Mdx mouse model of Duchenne Muscular Dystrophy: 8-week old Mdx animals (and healthy wildtype, age-matched controls) were given oral infusions of TY4 or vehicle (0.15 μg/g, twice a week) for eight weeks.

Mouse Scleroderma model: Wildtype mice received subcutaneous infusions of bleomycin sulfate for three weeks followed four weeks of biweekly oral administration of TY4, scramble or vehicle (0.15 μg/g, twice a week).

### Exercise Endurance Test

Mice were placed inside an Exer-6 rodent treadmill (Columbus Instruments) at a 5-degree elevation. At first the speed was increased by 5 m/min for 2 min, and the speed remained 10 m/min for 5 min. Then the speed was increased by 2 m/2 min until mice were exhausted. Exhaustion was defined as the inability of the mouse to return from the shock grid for 10 second.

### Western Blot

Protein extracts from mouse tissue were prepared by lysis in RIPA buffer (89900, Thermo Scientific) containing protease and phosphatase inhibitor (78442, Thermo Scientific). Protein samples (normalized value between 10-30 g) were separated for gel electrophoresis (NUPAGE 4%-12% Bis-Tris gel, NP0336 Thermo Fisher Scientific) and transferred to nitrocellulose membranes using Trans-Blot Turbo Transfer System, Bio-Rad. Proteins were detected with the following primary antibodies: p21 (ab109199, abcam) and GAPDH (3683S, Cell Signaling technology).

RNA Isolation and qPCR

Total RNA was extracted from mouse tissue using RNeasy plus kit (74136, QIAGEN) and Maxtract High density (129056, QIAGEN). cDNA was synthesized from RNA using High capacity cDNA reverse transcription kit (4368813, Applied Biosystems) according to the manufacture's protocol. Real time PCR (QuantStudio 12K Flex Real-Time PCR system; Thermo Fisher Scientific) was performed in triplicate using following TaqMan Gene Expression Assay probes; IL-6 (Mm00446190_m1), IL1-b (Mm00434228_m1), p21 (Mm00432448_m1) and analyzed by the ddCt method.

### RNA Sequencing

Cell and tissue RNA samples were sequenced at the Cedars-Sinai Genomics Core as described previously¹⁸. Total RNA were analyzed using an Illumina NextSeq 500 platform.

### Bone Marrow Cell Isolation, Macrophage Differentiation, and Activation

Femurs were isolated from 7-10-week-old Wistar Kyoto rats. Bone marrow was isolated by flushing with PBS (containing 1% FBS, 2 mM EDTA) then filtering through a 70 m mesh. Red blood cells were lysed with ACK buffer (A1049201, Invitrogen) and then resuspended in IMDM (Gibco) containing 10 ng/ml M-CSF (RP8643, Fisher Scientific) for plating. The media was exchanged every 2-3 days until day 7, at which point bone marrow-derived macrophages (BMDMs) were obtained. BMDMs were transfected with YRNAs (80 nM) using DharmaFect1. For LPS exposure, macrophages were pretreated with vehicle, TY4 (80 nM), TY4 scramble control (80 nM) for three hours followed by exposure to lipopolysaccharide (10 ng/ml; Cayman Chemical)

### Enzyme-Linked Immunosorbent Assay (ELISA)

IL-6 and BNP plasma levels were analyzed using following ELISA kit: Mouse IL-6 Quantikine ELISA Kit (M6000B, R&D systems), Mouse BNP EIA (EIAM-BNP-1, RayBiotech) according to manufacturer's instructions.

After overnight incubation, cells were washed and collected for RNA extraction using RNeasy plus kit.

Biodistribution of TY4 by qPCR

TY4 were administrated by both retro-orbital injection and blood and tissue were collected 30 min post-administration. Collected blood were allowed to clot for 2 hours at room temperature before centrifuging for 20 minutes at 2000×g.

### Statistical Analysis

Statistical parameters including the number of samples (n), descriptive statistics (mean and standard deviation), and significance are reported in the figures and figure legends. Differences between groups were examined for statistical significance using the Student's t-tests or analysis of variance. Differences with p values <0.05 were regarded as significant.

### REFERENCES

1 Marban, E. A mechanistic roadmap for the clinical application of cardiac cell therapies. Nat Biomed Eng 2, 353-361, doi:10.1038/s41551-018-0216-z (2018).
2 Smith, R. R. et al. Regenerative potential of cardiosphere-derived cells expanded from percutaneous endomyocardial biopsy specimens. Circulation 115, 896-908, doi:10.1161/CIRCULATIONAHA.106.655209 (2007).
3 McDonald, C. M. et al. Repeated intravenous cardiosphere-derived cell therapy in late-stage Duchenne muscular dystrophy (HOPE-2): a multicentre, randomised, double-blind, placebo-controlled, phase 2 trial. Lancet 399, 1049-1058, doi:10.1016/S0140-6736(22)00012-5 (2022).
4 Taylor, M. et al. Cardiac and skeletal muscle effects in the randomized HOPE-Duchenne trial. Neurology 92, e866-e878, doi:10.1212/WNL.0000000000006950 (2019).
5 Ibrahim, A. G., Cheng, K. & Marban, E. Exosomes as critical agents of cardiac regeneration triggered by cell therapy. Stem Cell Reports 2, 606-619, doi:10.1016/j.stemcr.2014.04.006 (2014).
6 Walravens, A. S. et al. Mechanistic and therapeutic distinctions between cardiosphere-derived cell and mesenchymal stem cell extracellular vesicle non-coding RNA. Sci Rep 11, 8666, doi:10.1038/s41598-021-87939-9 (2021).
7 de Couto, G. et al. Exosomal MicroRNA Transfer Into Macrophages Mediates Cellular Postconditioning. Circulation 136, 200-214, doi:10.1161/CIRCULATIONAHA.116.024590 (2017).
8 Cambier, L. et al. Y RNA fragment in extracellular vesicles confers cardioprotection via modulation of IL-10 expression and secretion. EMBO Mol Med 9, 337-352, doi:10.15252/emmm.201606924 (2017).
9 Cambier, L. et al. Angiotensin II-Induced End-Organ Damage in Mice Is Attenuated by Human Exosomes and by an Exosomal Y RNA Fragment. Hypertension 72, 370-380, doi:10.1161/HYPERTENSIONAHA.118.11239 (2018).
10 Huang, F. et al. Exosomally derived Y RNA fragment alleviates hypertrophic cardiomyopathy in transgenic mice. Mol Ther Nucleic Acids 24, 951-960, doi:10.1016/j.omtn.2021.04.014 (2021).
11 Egli, M. & Manoharan, M. Re-Engineering RNA Molecules into Therapeutic Agents. Acc Chem Res 52, 1036-1047, doi:10.1021/acs.accounts.8b00650 (2019).
12 Mathews, D. H. et al. Incorporating chemical modification constraints into a dynamic programming algorithm for prediction of RNA secondary structure. Proc Natl Acad Sci USA 101, 7287-7292, doi:10.1073/pnas.0401799101 (2004).
13 Shah, S. J. et al. Research Priorities for Heart Failure With Preserved Ejection Fraction: National Heart, Lung, and Blood Institute Working Group Summary. Circulation 141, 1001-1026, doi:10.1161/CIRCULATIONAHA.119.041886 (2020).
14 Hieda, M. et al. Increased Myocardial Stiffness in Patients With High-Risk Left Ventricular Hypertrophy: The Hallmark of Stage-B Heart Failure With Preserved Ejection Fraction. Circulation 141, 115-123, doi:10.1161/CIRCULATIONAHA.119.040332 (2020).
15 Sweeney, M., Corden, B. & Cook, S. A. Targeting cardiac fibrosis in heart failure with preserved ejection fraction: mirage or miracle? EMBO Mol Med 12, e10865, doi:10.15252/emmm.201910865 (2020).
16 Sabbah, M. S. et al. Obese-Inflammatory Phenotypes in Heart Failure With Preserved Ejection Fraction. Circ Heart Fail 13, e006414, doi:10.1161/CIRCHEARTFAILURE.119.006414 (2020).
17 Schiattarella, G. G. et al. Nitrosative stress drives heart failure with preserved ejection fraction. Nature 568, 351-356, doi:10.1038/s41586-019-1100-z (2019).
18 Ibrahim, A. G. E. et al. Augmenting canonical Wnt signalling in therapeutically inert cells converts them into therapeutically potent exosome factories. Nat Biomed Eng 3, 695-705, doi:10.1038/s41551-019-0448-6 (2019).

### Example 15

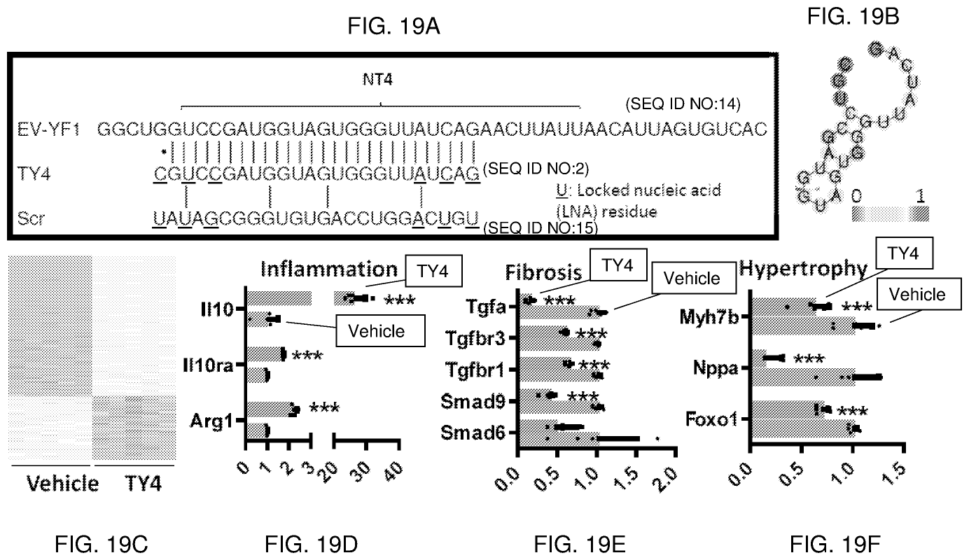

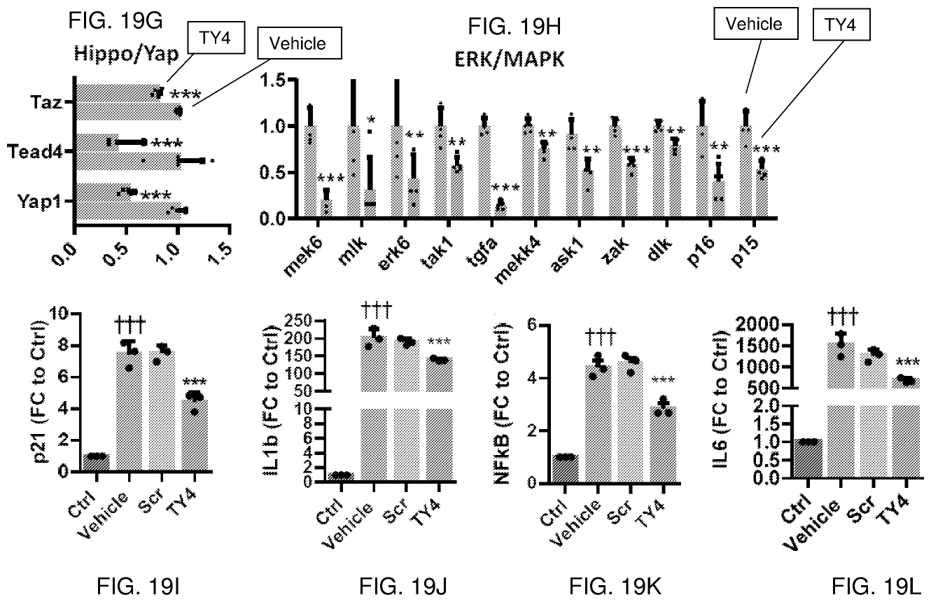

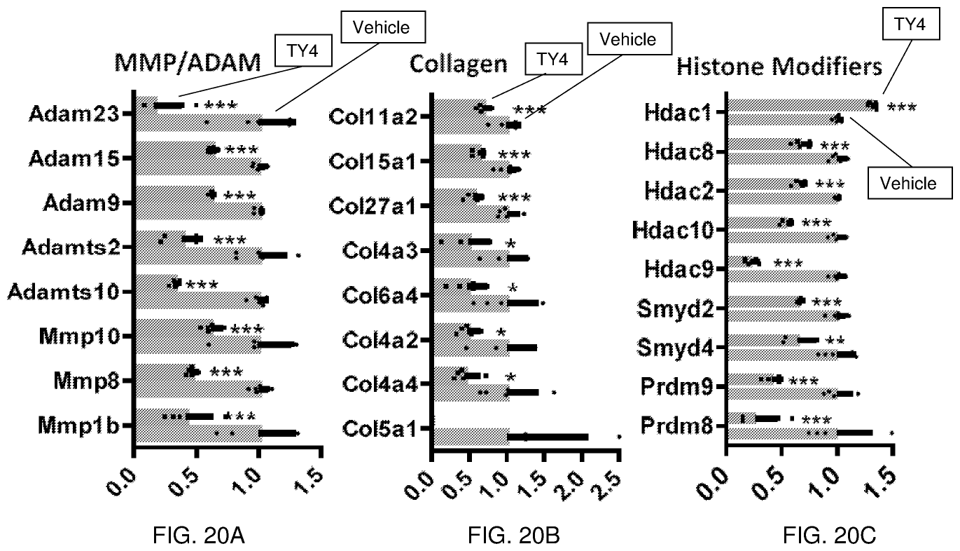

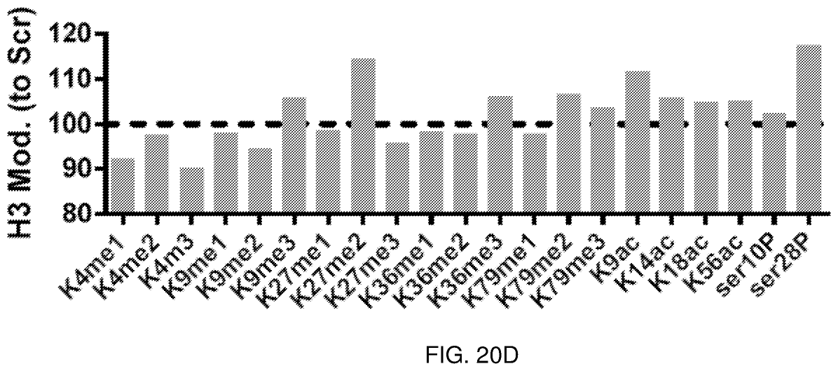

As discussed in Example 3 above, TY4 was derived from EV-YF1, where TY4 contains a point mutation (G to C) at its 5′ end, as well as six locked nucleic acid (LNA) modifications at the residues underlined (FIG. 19A). As a control, a scrambled version (bottom row) containing the same nucleotides as TY4 but in random order, as well as 6 LNAs at the same positions, was created. In silico prediction¹² of TY4 structure demonstrated an interrupted stem loop structure and an external loop formed by the 3′ and 5′ ends (FIG. 19B). The profound transcriptomic changes induced by TY4 in bone marrow-derived macrophages, shown in FIG. 4B, is also represented in a different format in FIG. 19C. In addition to elevating IL10 expression, as seen in FIG. 4C, notable changes in expression of genes in pathways relevant to inflammation (FIG. 19D), fibrosis (FIG. 19E; FIGS. 20A, 20B), and hypertrophy (FIG. 19F) were also observed. Analysis of potential upstream regulators identified TY4-induced suppression of the Hippo/Yap pathway (FIG. 19G) and Erk/Map Kinase pathways (FIG. 19H). These broad changes were further manifested by altered histone-modifying gene expression including deacetylases and methyltransferases (FIG. 20C), the consequences of which were evident in a modified histone ELISA array (FIG. 20D). These findings revealed effects on master regulators and downstream signals governing inflammation, hypertrophy, and fibrosis. As further evidence of fundamental effects, macrophages exposed to TY4 exhibited suppression of the cell cycle inhibitor p21 (FIG. 19I), inflammasome signaling (IL-1b; FIG. 19J), and inflammation (NFkB and IL6; FIGS. 19K, 19L, respectively) compared to vehicle or scramble.

### Example 16

This non-limiting example shows the therapeutic effect of TY4 in a model of heart failure with preserved ejection fraction (HFpEF), as described in Example 5, and provides additional analysis of the results.

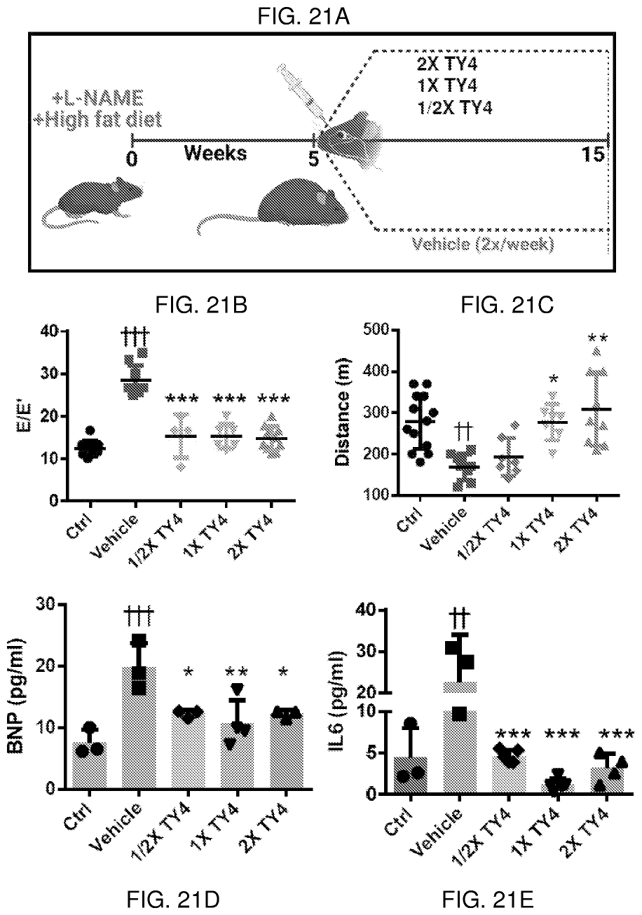

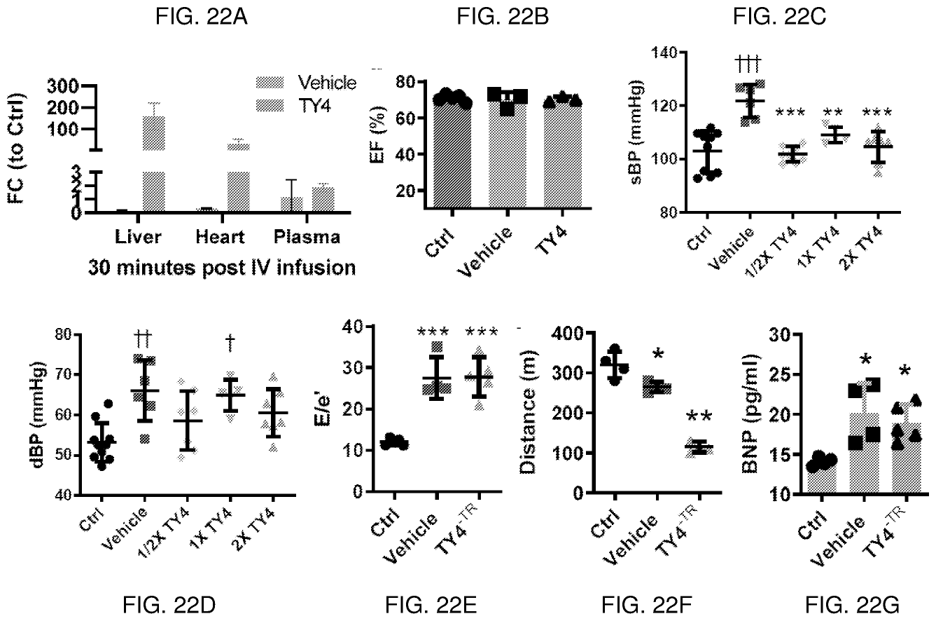

Heart failure with preserved ejection fraction (HFpEF) is a systemic illness¹³, refractory to conventional therapy, and marked by cardiac hypertrophy¹⁴, fibrosis¹⁵, and inflammation¹⁶. Given that TY4 appears to inhibit all these processes, TY4 in a mouse model of HFpEF induced by high-fat diet and L-NAME¹⁷ was studied. After 5 weeks, when signs of HFpEF are evident, TY4 was administered intravenously (IV) at various dosing frequencies (bi-weekly, weekly, and twice weekly; 0.15 μg TY4/g body weight per dose) and the results were compared to IV vehicle (FIG. 21A). Thirty minutes after a single IV dose, TY4 was detectable above background in liver and heart (FIG. 22A). TY4 had salubrious effects on various key manifestations of HFpEF, including diastolic dysfunction (the higher the echocardiographic parameter E/e′, the worse the heart function; FIG. 21B), exercise endurance (FIG. 21C) and circulating BNP (a marker of heart failure; FIG. 21D) and IL6 (an inflammatory cytokine; FIG. 21E). After 10 weeks of dosing, all groups had a normal ejection fraction (as shown by representative samples from control, vehicle and TY4-treated groups), as expected in HFpEF (FIG. 22B). The groups receiving TY4 also had lower blood pressures (with more consistent reductions in systolic than diastolic blood pressure; FIGS. 22C and 22D). While all dosing regimens were efficacious on diastolic function and serum biomarkers, exercise endurance improved only with once- or twice-weekly TY4 (FIG. 21C). Administration of TY4 without a transfection reagent abrogated its bioactivity (no changes in diastolic function [FIG. 22E], exercise endurance [FIG. 22F], or circulating BNP levels [FIG. 22F]). Therefore, TY4 reverses the systemic and cardiac manifestations of HFpEF.

Nonspecific induction of the innate immune response can modestly improve cardiac function. Because injection of TY4 might activate innate immunity, we compared TY4 not to vehicle, but rather to a scrambled version of TY4 with the same nucleotide content (see FIG. 19A. FIG. 32, left panel, shows that EF was similar and normal in all groups tested. FIG. 32, right panel, shows that mice that received Scr developed diastolic dysfunction similar to that with vehicle (compare with FIG. 8D), unlike mice which received TY4. Thus, the scramble (Scr) failed to correct diastolic dysfunction in HFpEF mice, while TY4 did.

### Example 17

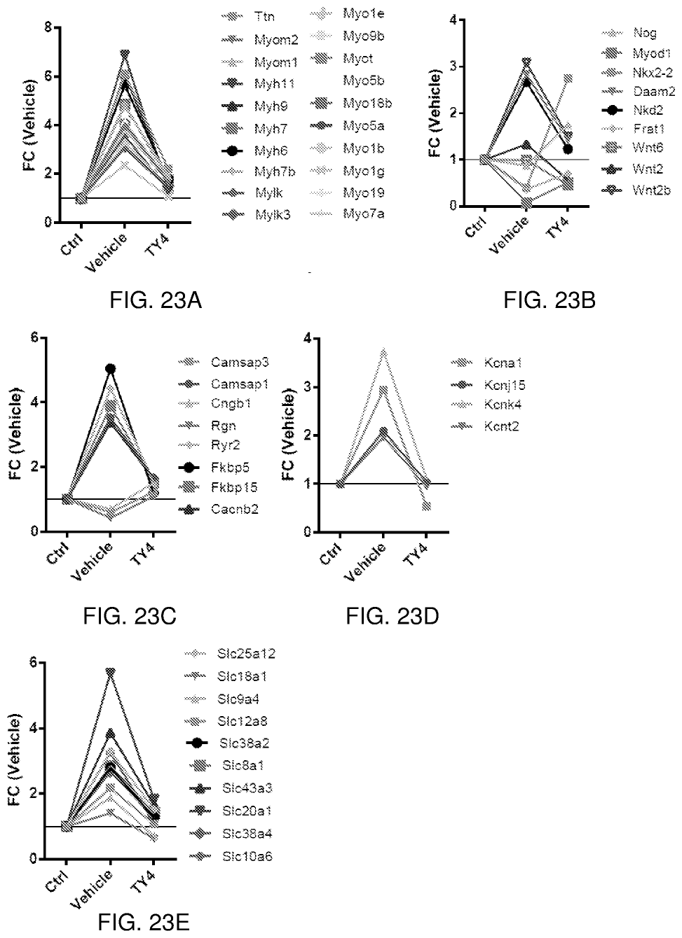

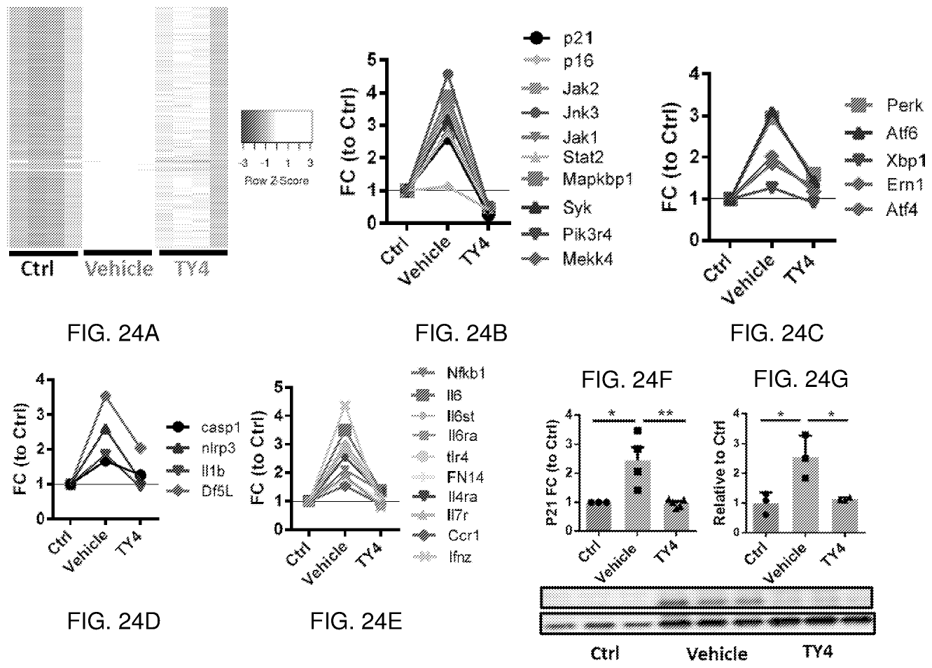

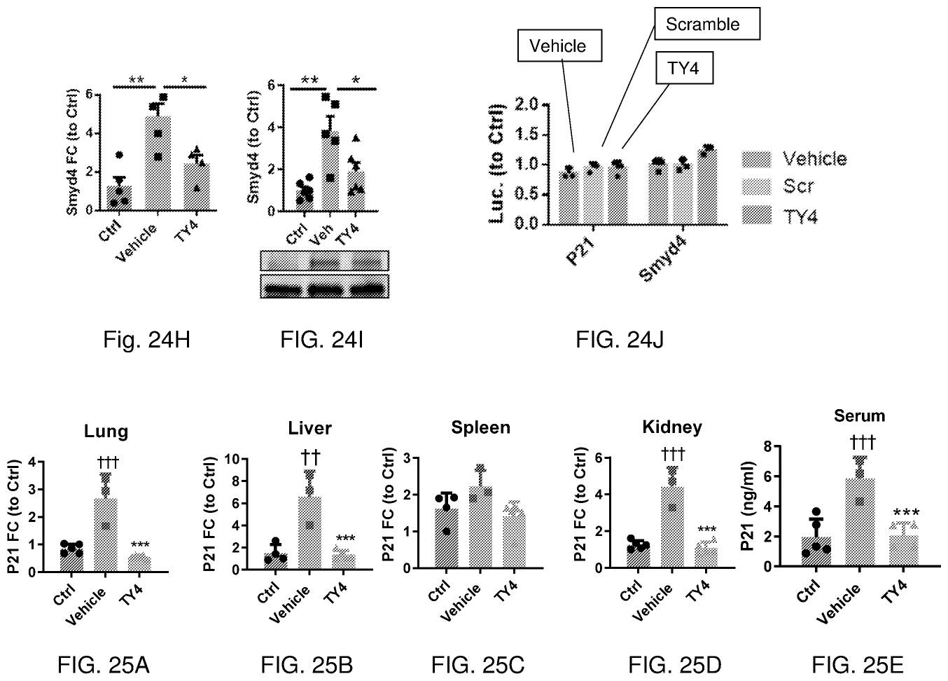

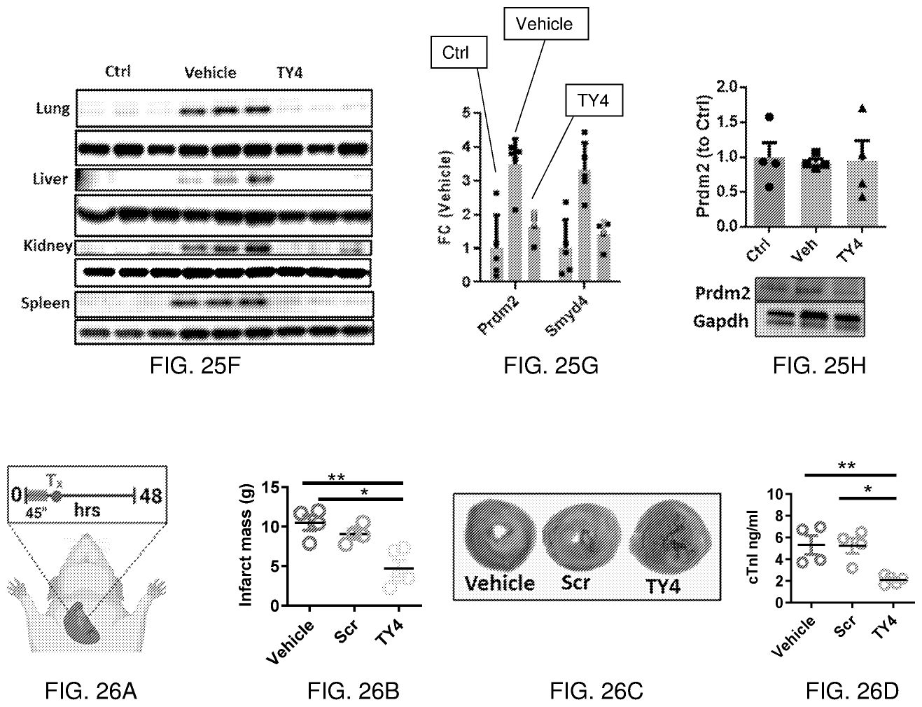

Underlying the benefits of TY4 in HFpEF mice, RNA sequencing of cardiac tissue showed TY4 broadly reset gene expression (heat maps, FIG. 24A), which included normalization of gene families relevant to structure (FIG. 23A), canonical Wnt signaling (FIG. 23B), calcium handling (FIG. 23C), electrical repolarization (FIG. 23D), and membrane transport proteins of the solute carrier family (FIG. 23E). Normalization of upstream regulators including the p21/Map kinase pathway (FIG. 24B) was associated with reductions in key cell stress pathways including intraluminal ER stress sensors (FIG. 24C), inflammasome (FIG. 24D) and inflammatory mediators (FIG. 24E). TY4-induced reductions in p21 gene expression, previously identified in macrophages (FIG. 19I), were even more profound in HFpEF heart (FIG. 24F) and confirmed at the protein level (FIG. 24G). p21 was suppressed not only in heart, but also in the lung, liver, spleen, kidney, and serum of TY4-treated animals (transcripts, FIG. 25A-25D; protein, FIGS. 25E, 25F). Among the chromatin modifying genes suppressed in TY4-treated macrophages (FIG. 20C) was Smyd4, a potentiator of p21-mediated stress. Consistently transcripts of Smyd4, and another histone methyltransferase related to p21 signaling (Prdm2) suppressed in TY4-treated HFpEF hearts were found (FIG. 25G). Of the two, however, only Smyd4 was suppressed at the protein level (FIG. 24I; cf. Prdm2, FIG. 25H). As the size of TY4 is comparable to that of micro RNAs and siRNAs, we tested to see if TY4 binds to the 3′ untranslated region (UTR) to transcriptionally regulate these genes similarly. Results from a luciferase 3′ UTR assay in HEK293 cells indicate that TY4 does not mediate transcriptional repression of p21 or Smyd4, such that another mechanism must be at play (FIG. 24J).

### Example 18

Examples 18-21 are non-limiting example showing that TY4 is efficacious in models of myocardial infarction, muscular dystrophy and scleroderma.

Given the dramatic disease-modifying effects of TY4 in HFpEF, a chronic illness, whether TY4 can exert acute cardioprotective effects was investigated. In a rat model of myocardial infarction (FIG. 26A), a single IV dose of TY4 (0.15 μg/g) 20 minutes after reperfusion decreased circulating cardiac troponin levels (FIG. 26B) and scar size (FIGS. 26C, 26D) compared to vehicle or scramble controls.

### Example 19

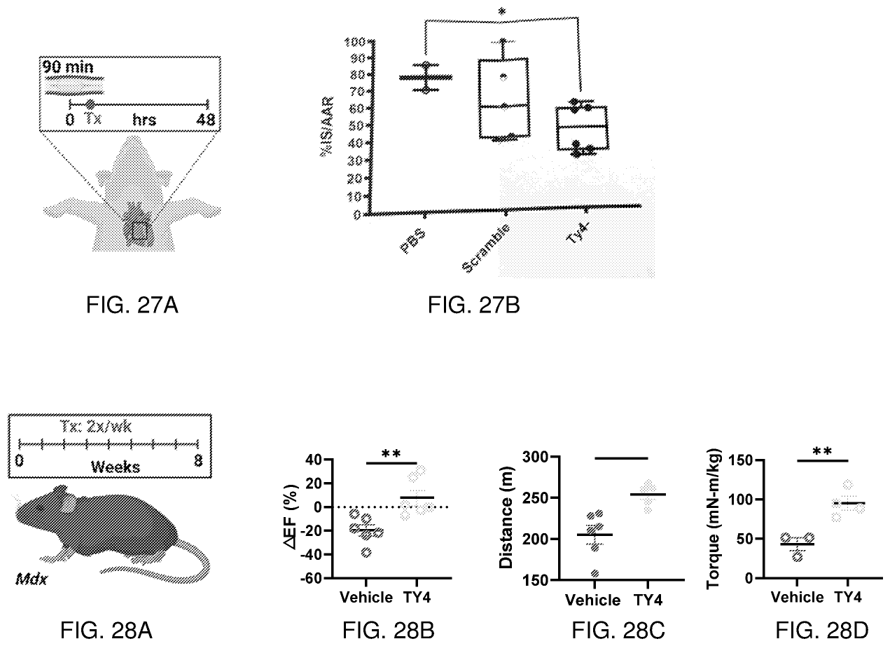

To expand clinical relevance, a porcine model of myocardial infarction was studied (FIG. 27A). As in rats, a single IV infusion of TY4 after reperfusion led to preserved global function (ejection fraction, FIG. 27B) and reduced scar size, 48 hours post myocardial infarction, compared to vehicle or scramble groups.

### Example 20

This non-limiting example shows the therapeutic effect of TY4 in a model of Duchenne muscular dystrophy, as described in Example 13, and provides additional analysis of the results.

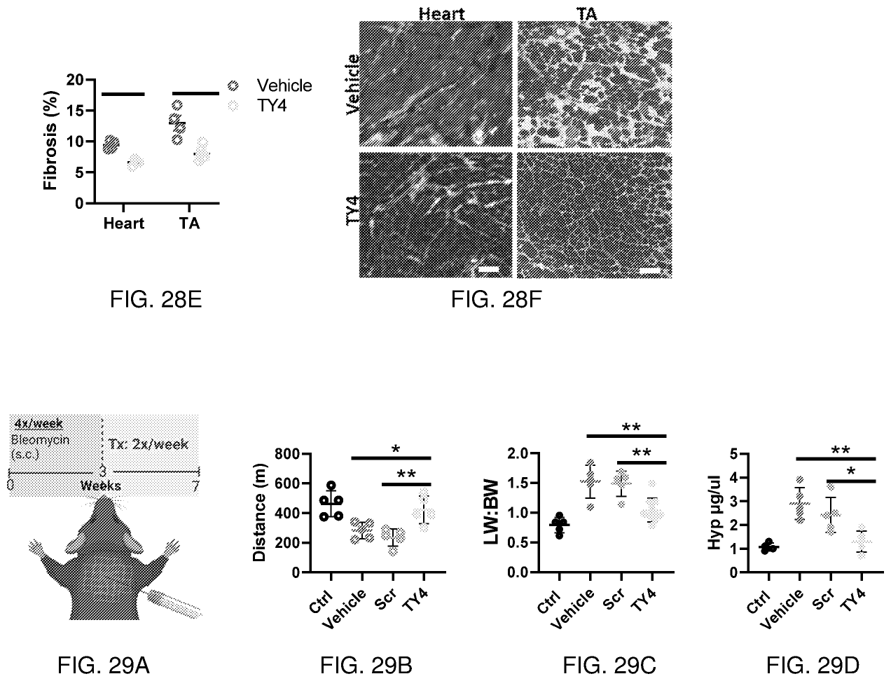

DMD is a genetic disease inducing muscle loss, inflammation and fibrosis in heart and skeletal muscle. In the mdx mouse model of DMD, twice-weekly IV infusions of TY4 (FIG. 28A) improved ejection fraction (FIG. 28B), and increased exercise endurance compared to control or scramble (FIG. 28C; see FIGS. 16A and 16C). Analysis of the soleus muscle demonstrated attenuated fibrosis (FIGS. 28E, 28F) and increased tetanic force generation (FIG. 28D; see FIGS. 17A and 17C).

### Example 21

Scleroderma is an autoimmune disorder marked by progressive skin thickening and fibrosis of skin, heart and lung. To model scleroderma, animals were injected with bleomycin intradermally over the course of 3 weeks, followed by 4 weeks of TY4, scramble, or vehicle (FIG. 29A). TY4 reduced skin thickening and skin fibrosis as measured by hydroxyproline content (FIG. 29D), improved exercise endurance (FIG. 29B), normalized lung weight to body weight ratio (FIG. 29C), and attenuated weight loss.

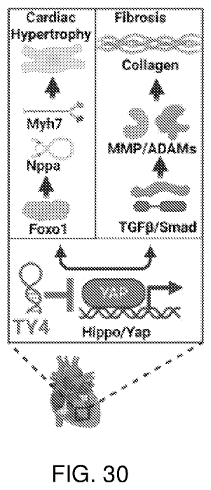

Thus, Examples 18-21 demonstrate that TY4 is highly effective against pathological processes as diverse as ischemia, myodegeneration, and autoimmunity. Further, these examples are consistent with a model by which TY4 suppresses both hypertrophic and pro-fibrotic cascades (FIG. 30).

### Example 22

This non-limiting example shows an in vitro assay for assessing TY4 activity, and demonstrates that pre-treatment of bone marrow-derived macrophages by TY4 attenuates LPS-mediated pro-inflammatory activation as shown by pro-inflammatory gene expression.

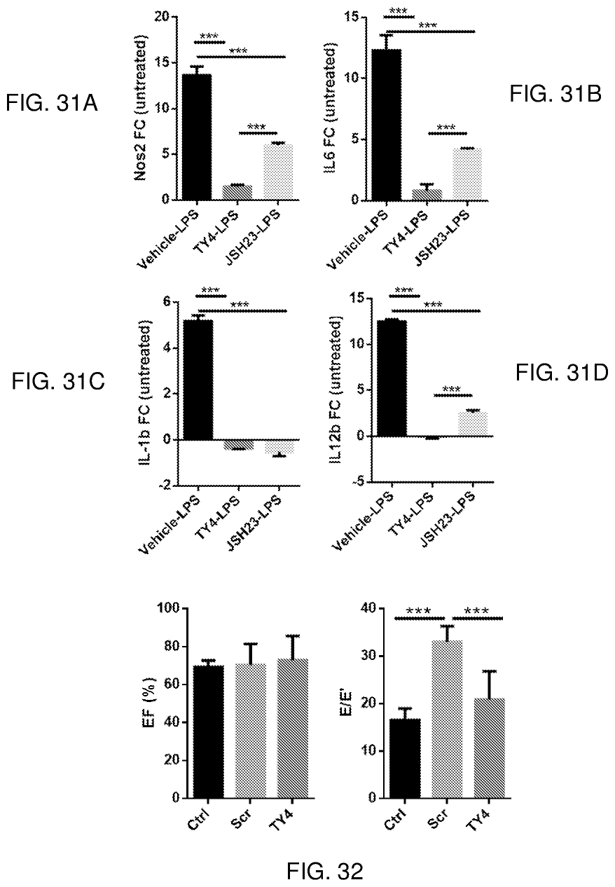

TY4 activity was assessed using a primary BMDM activation assay. BMDM were exposed to lipopolysaccharide (LPS) to stimulate inflammatory responses. Mononuclear cells were isolated from the femurs of wild type mice. BMDM were generated by exposure to macrophage colony stimulating factor (MCSF) for five days, then co-culturing with 80 nM TY4, vehicle (saline; negative control), or JSH23 (a small molecule inhibitor of NFkB; positive control) for 3 hours. BMDM were then exposed to LPS, and RNA was isolated 12 hours later for gene expression analysis. Exposure of LPS-stimulated BMDM to TY4 promoted secretion of IL10 and blunted inflammatory markers NOS2 (FIG. 31A), IL6 (FIG. 31B), IL1b (FIG. 31C), and IL2b (FIG. 31D), compared to the vehicle group.

### Example 23

This non-limiting example shows the therapeutic effect of TY4 in a model of HCM as described in Example 4, and provides additional analysis of the results. This non-limiting example shows that intravenous TY4 reduces cardiac hypertrophy and systemic inflammation, while improving exercise endurance in a mouse model of hypertrophic cardiomyopathy.

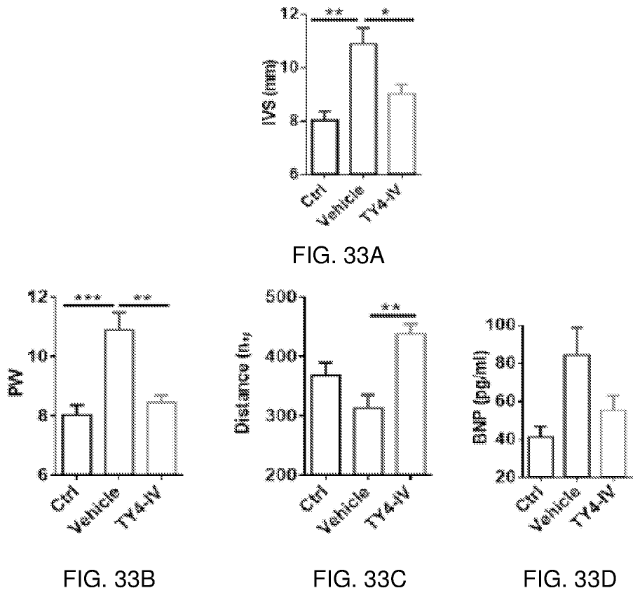

To assess the disease-modifying bioactivity of TY4 in a target indication, HCM, transgenic cTnI^{146Gly} mice, which model a particularly lethal human HCM mutation, were used, as described in Example 4. Echocardiography revealed marked reductions in interventricular septal (IVS) thickness (FIG. 33A), reduced outflow velocity as measured by pulse wave doppler (PW, FIG. 33B), increased exercise tolerance (distance, FIG. 33C), and reduced levels of a circulating biomarker of heart failure (HF) (BNP; FIG. 33D). All of these parameters are disease-relevant: IVS thickness as a direct measurement of hypertrophy, PW as an indication of how well the ventricle can eject blood, Distance as an integrative measure of overall physical capacity, and BNP as a biomarker of HF. Thus, TY4 is highly therapeutic in a model of HCM.

### Example 24

This non-limiting example shows IV and oral formulations of TY4.

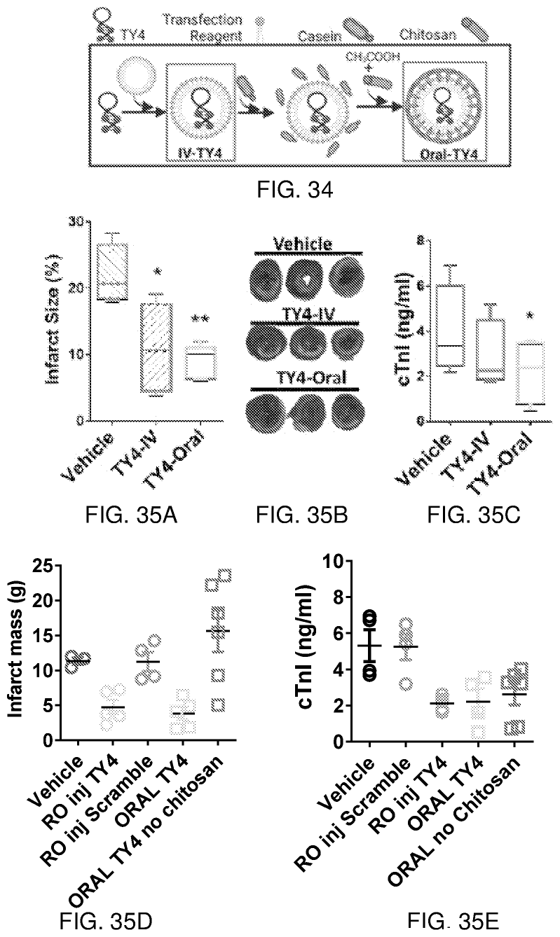

Research-grade TY4 was synthesized commercially by Integrated DNA Technologies (IDT, Inc.). For transfection and intravenous (IV) formulation, the RNA oligo was mixed with Dharmafect (Perkin Elmer), a cationic lipid for the transfection of small RNAs. TY4 was mixed with Dharmafect at a ratio of 20 ng RNA per 1 μL of Dharmafect and filled to a final volume of 100 μL of serum-free media. The solution was vortexed for 15 seconds (three total agitations) and left at room temperature for five minutes for RNA-transfection complexes to form (FIG. 34, IV-TY4). This comprised the in vitro formulation, as well as the IV formulation of TY4.

For oral formulation, the TY4-Dharmafect complex was further encased in a casein-chitosan micelle. This was achieved by first adding 5% bovine casein solution to the TY4-Dharmafect solution at a volume ratio of 1:10 and incubating at room temperature for 15 minutes. To precipitate the casein and form chitosan-casein micelles encapsulating TY4-Dharmafect, an equal volume of 0.1% acetic acid solution/0.2% chitosan solution was added. The mixture was left to incubate at room temperature for one hour and represented the oral formulation (FIG. 34, Oral-TY4).

### Example 25

This non-limiting example shows therapeutic effects of orally administered TY4 in a model of MI.

To assess the effectiveness of oral delivery of a therapeutic nucleic acid, myocardial infarction was modeled in mice by open-chest occlusion of the left anterior descending coronary artery for 45 min, followed by reperfusion. The chest was then closed. Twenty min after reperfusion, mice were given either vehicle or an IV composition made up of a lipid-encapsulated therapeutic RNA, or oral composition comprising a therapeutic RNA encapsulated in a lipid micelle and coated with casein-chitosan, as provided for herein. The non-limiting example of a nucleic acid payload use here was TY4. Hearts were excised 48 hours post-MI and infarct size (IS) quantified histologically.

FIG. 35A shows pooled data related to infarct size. As shown, both IV and oral compositions resulted in reduced infarct size. Notably, the oral delivery of TY4 shows an enhanced reduction of infarct size. Representative left ventricular sections for each group are shown in FIG. 35B, with the orally-treated group showing markedly less infarct scarring. As a marker of cardiac injury, FIG. 35C shows that orally administered TY4 yielded a significant decrease as compared to control. Taken together, the data for histology and troponin I are mutually-reinforcing in showing the cardioprotective efficacy of orally delivered TY4.

Further building on those finds, additional comparative analysis is shown in FIGS. 35D and 35E. FIG. 35D shows IV injection of TY4 (or a scrambled version thereof) or orally administered TY4 housed in compositions according to embodiments disclosed herein (e.g., micelles coated with casein-chitosan). An additional group here includes oral TY4 encapsulated in a micelle that is coated with casein alone (no chitosan). The data of FIG. 35D reinforce the findings discussed above with respect to oral delivery of a therapeutic RNA, but also demonstrate that, according to preferred embodiments, a lipid micelle is coated with both casein and chitosan. The test group on the right of FIG. 35D is the RNA-encapsulated micelles coated with casein only. Infarct size for that group was notably increased, indicative of a reduced bioavailability of the TY4, believed to be due to less robust protection for the composition in the low-acid environment of the stomach. FIG. 35E shows less of a drop off in efficacy, as related to measuring cardiac troponin I, though the casein-only formulation appears to at least trend towards elevated concentrations (less therapeutic effect). Taken together, these data support the cardioprotective efficacy of orally delivered TY4, using a casein-chitosan coating, as provided for herein in several embodiments.

### Example 26

This non-limiting example shows oral formulations of TY4 encapsulated in lipid micelles and coated with a casein-chitosan complex improve symptoms of scleroderma.

The model of scleroderma described in Example 21 was used to treat animals with compositions configured for oral delivery of TY4, as provided for herein. TY4 was encapsulated in a lipid micelle coated with casein-chitosan and delivered orally. Oral delivery (in the same delivery composition) of a scrambled RNA sequence was used as a control.

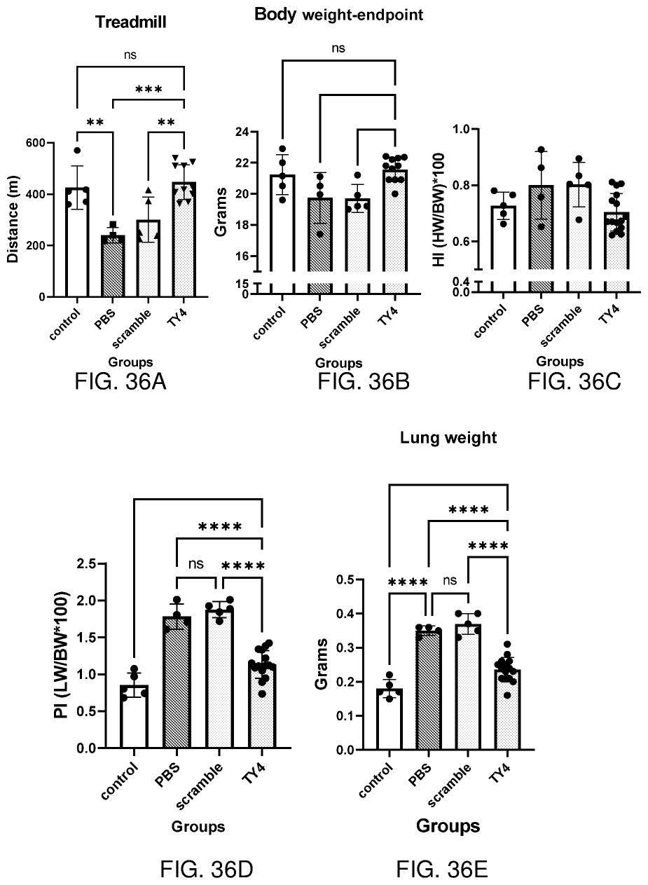

FIG. 36A shows data related to endurance testing on a treadmill. As shown, oral delivery of the therapeutic RNA resulted in full return of endurance to that of untreated control mice, PBS control and scrambled RNA sequence controls each showed significant reductions in endurance. FIG. 36B shows that oral delivery of the therapeutic RNA allowed mice to maintain body weight such that it was not significantly different from control. FIG. 3C shows a heart index (HI) that relates the heart weight to the body weight of the mice in each group. As expected from the reduction in body weight with the non-therapeutic groups, these groups exhibited an elevated HI. Also, an increase in heart weight (e.g., due to fibrosis) could also account for an aspect of the increased HI. Likewise, when measuring pulmonary index (PI), which indexes lung weight as a function of body weight, the orally delivered therapeutic RNA results in significantly reduced PI as compared to the non-therapeutic groups (though the PI was still elevated over control). FIG. 36E shows the lung weight data alone, which corresponds to the PI data.

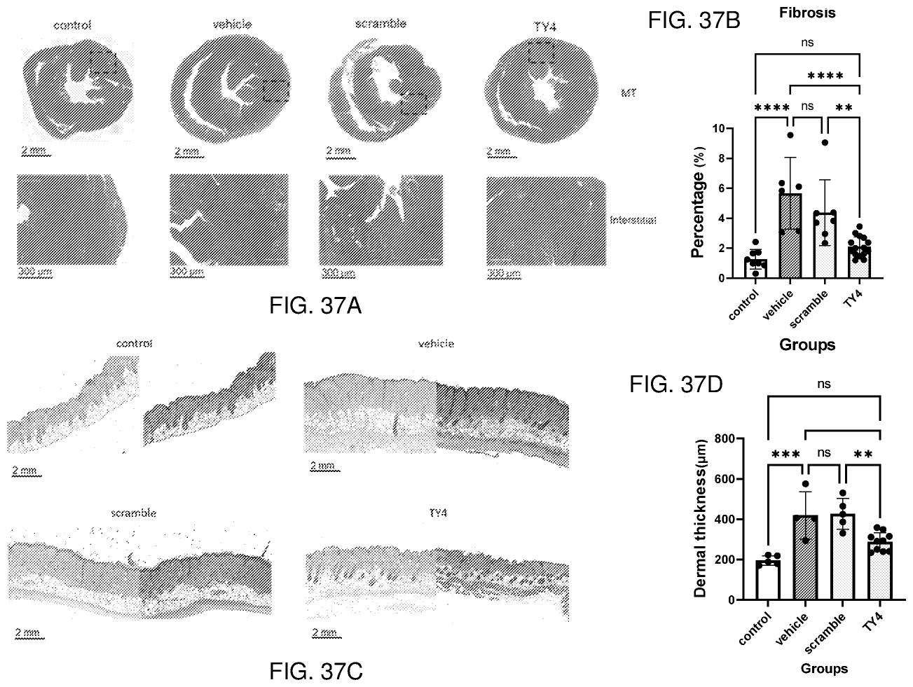

Turning specifically to cardiac measures, FIG. 37A shows histology data related to fibrosis. The top row shows representative tissue stains with dashed boxes corresponding to the enlarged view provided in the second row. The orally delivered therapeutic RNA shows a far reduced fibrosis of the tissue. FIG. 37B shows the quantification of fibrosis for each group, indicating that oral delivery of the therapeutic RNA results in significantly reduced cardiac fibrosis. Turning to symptoms of scleroderma that impact the skin, FIGS. 37C and 37D relate to fibrosis of the skin. FIG. 37C show histology data related to the skin with the upper left showing control skin, upper right showing vehicle control, lower left showing the scrambled RNA, and lower right showing the orally delivered TY4 RNA. The orally delivered TY4 RNA, as a non-limiting example of a therapeutic RNA, resulted in visibly less thickening/fibrosis of the skin as compared to the non-treatment groups. FIG. 37D confirms this with a graph of derma thickness for each group, with orally delivered therapeutic RNA resulting in dermal thickness that is not significantly different from control.

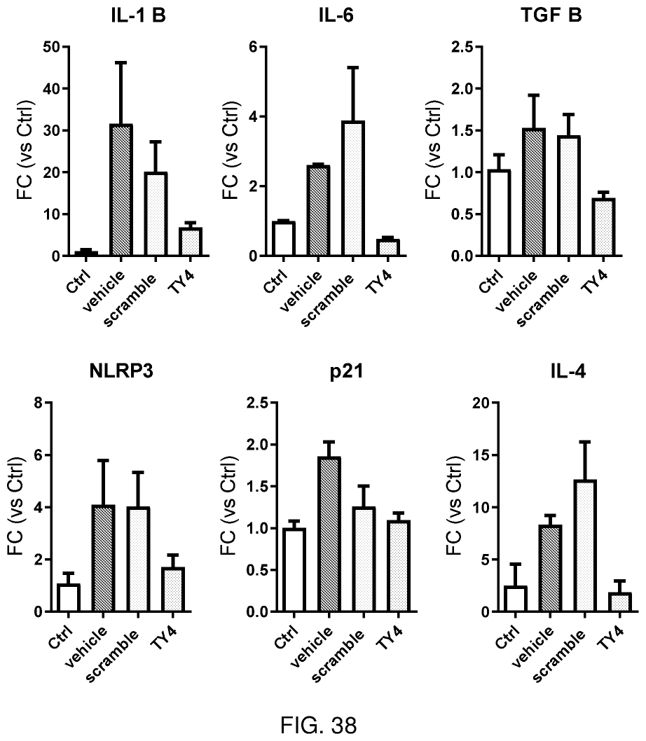

Additional investigation was undertaken with respect to the expression levels of various inflammatory cytokines. FIGS. 38A-38F show qPCR data related to quantification of IL1-B (FIG. 38A), IL-6 (FIG. 38B), TGF beta (FIG. 38C), NLRP3 (FIG. 38D), p21 (38E), and IL-4 (FIG. 38F). Each of these cytokines was equivalent to or showed only modest increases in expression when the orally delivered TY4 RNA was administered. These data thus support the efficacy of oral delivery of TY4 RNA to reduce fibrosis and/or inflammatory conditions, such as those secondary or symptomatic of scleroderma.

### Example 27

This non-limiting example shows the therapeutic effect of TY4, in particular orally administered TY4, in a model of heart failure with preserved ejection fraction (HFpEF).

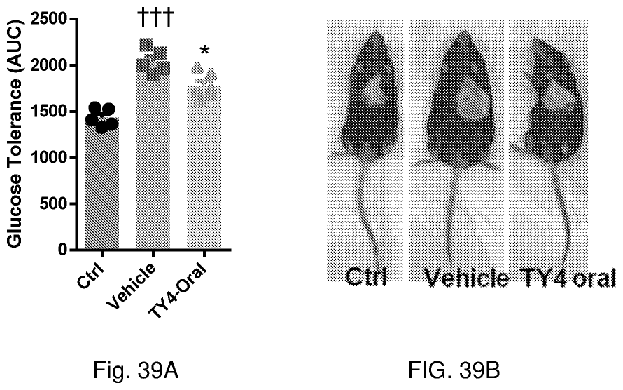

The therapeutic effects of orally administered TY4 in a model of HFpEF described in Example 5 were further studied. Animals were evaluated for circulating blood glucose concentrations. As shown in FIG. 39A, treatment with vehicle alone results in elevated blood glucose concentration as compared to control. Oral administration of TY4 according to embodiments disclosed herein results in significant reductions in circulating blood glucose levels, which can be related to the obesity associated with HFpEF. FIG. 39B shows fat accumulation in the vehicle-treated mouse. As shown with th >use on the far right, which received oral administration of TY4, there is a reduction in fat accumulation.

These data in combination with Examples 5 further reinforce that orally delivered compositions TY4 can effectively treat HFpEF. In several embodiments, the compositions and methods provided for herein, when administered orally, can effectively reduce inflammation and/or fibrosis that are the result of, or a symptom of a disease.

### Example 28

This non-limiting example shows oral formulations of TY4 encapsulated in lipid micelles and coated with a casein-chitosan complex improve symptoms of muscular dystrophy.

To determine the effects of orally delivered TY4, twelve-fourteen-month-old female mdx mice were fed TY4 (0.15 μg/g body weight) or vehicle twice-weekly for 8 weeks. Cardiac and skeletal muscle function were measured prior to feeding (i.e., baseline) and at the 8-week study endpoint. Hearts and tibialis anterior (TA) muscles were dissected and processed for Masson's trichrome staining to quantify interstitial fibrosis.

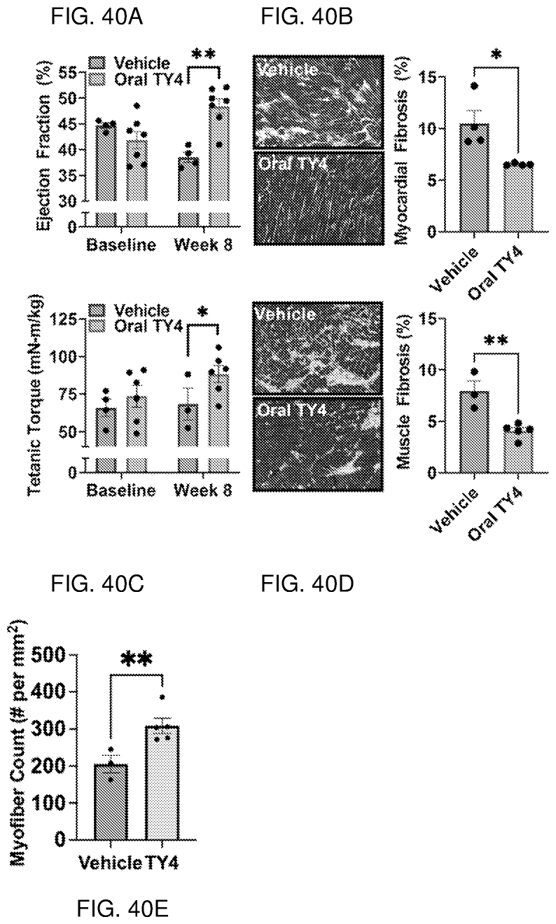

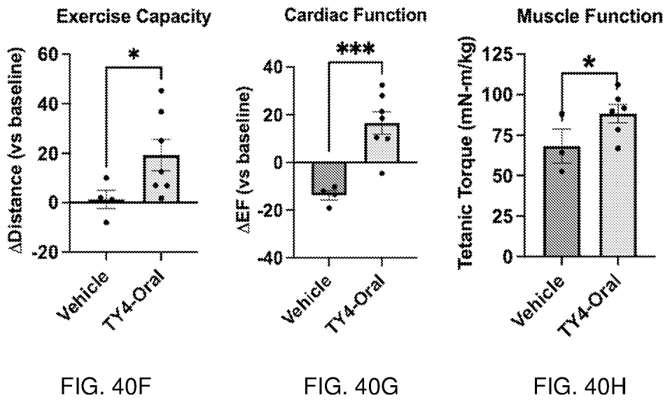

As shown in FIG. 40A, transthoracic echocardiography on lightly anesthetized mdx mice was performed to measure left ventricular ejection fraction (EF). At baseline, no differences were detected between groups. After 8 weeks, mdx receiving the orally administered example of a therapeutic RNA, TY4, had higher EF relative to vehicle control, which declined during the study period. As shown in FIG. 40B, Masson's trichrome micrographs and pooled data (right subpanel) show mdx mice receiving orally administered TY4 had less myocardial fibrosis than vehicle control mice. Turning to in vivo muscle function of the anterior crural muscles, of which the tibialis anterior (TA) produces ˜85% of the torque output for this muscle group, was recorded by attaching the foot of the mdx mouse to an aluminum shoe (which was attached to the servomotor of a force transducer) and stimulating the left common peroneal nerve. Tetanic torque was recorded at 200 Hz. At baseline, no differences were detected between groups (see FIG. 40C). After 8 weeks, mdx mice receiving orally administered TY4 produced more torque than vehicle control mice. FIG. 40D relates to muscle fibrosis and shows Masson's trichrome micrographs and pooled data (right subpanel). These data indicate that mdx mice receiving orally administered TY4 had less muscle fibrosis than vehicle control mice. FIG. 40E summarizes physiological data collected that demonstrates that there is a greater myofiber count (per mm²).

Functional data that were collected further support the efficacy of orally administered TY4 by way of delivery using a lipid micelle encapsulating the therapeutic RNA and coated with a casein-chitosan complex. FIG. 40E shows that in addition to the decreased interstitial fibrosis (FIGS. 40C, 40D) orally administered TY4 by way of delivery using a lipid micelle encapsulating the TY4 boosted the number of myofibers in the TA. This physiologic effect was confirmed in functional assays (FIGS. 40F-40H) which show enhance exercise capacity, cardiac function and muscle function, respectively, in animals that received oral administration of TY4 by way of delivery using a lipid micelle encapsuling the therapeutic RNA. Two-way ANOVA or an independent t-test was used to determine statistical significance between groups. *P<0.05, **P<0.01. Data are represented as mean±SEM.

These data further support the efficacy of orally administered TY4 by way of delivery using a lipid micelle encapsuling TY4 and coated with a casein-chitosan complex. In several embodiments, such compositions to deliver TY4 via an oral administration result in increased bioavailability of the RNA, which in turn leads to enhanced therapeutic outcomes for diseases hallmarked by inflammation and/or fibrosis, such as muscular dystrophy.

### Example 29

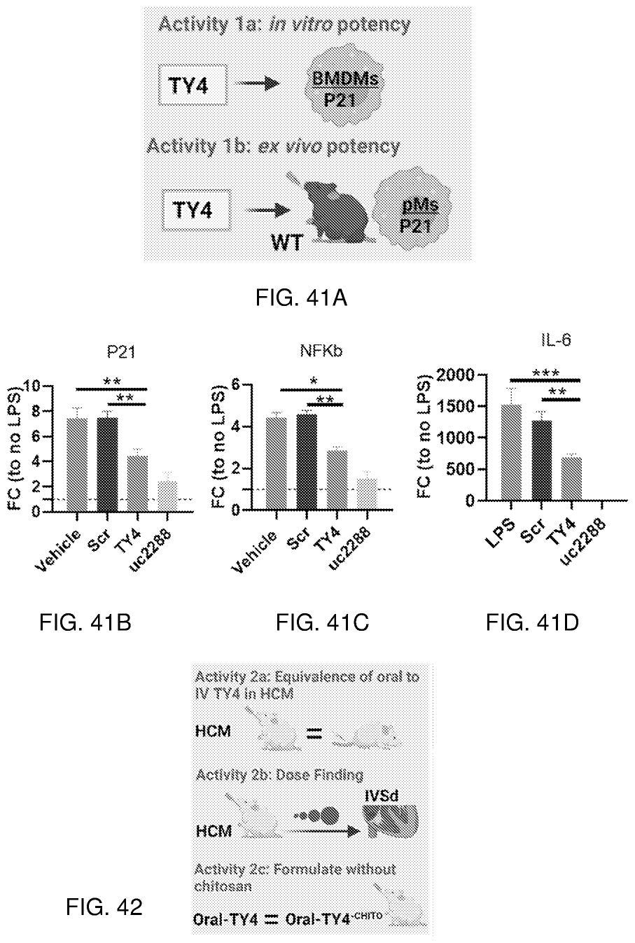

This non-limiting example shows an in vitro potency assay for TY4 in mouse bone marrow derived macrophages (FIG. 41A).

### Experimental Design

Based on mechanistic investigation, TY4 exerts its therapeutic effects at least partly through targeting pro-fibrotic pathways including p21 (FIGS. 24F, 24G). This occurs as a direct consequence of TY4's suppression of Smyd4, an HMT whose activity is known to upregulate p21. Furthermore, p21 suppression recapitulates the immunomodulatory effects of TY4 as shown by suppression of p21, NFkB, and IL6. Therefore, an in vitro and ex vivo potency assays are developed to confirm the bioactivity of various TY4 batches and formulations. Preclinical studies revealed TY4 leads to downregulation of p21 in the plasma of injected animals (FIG. 25E). Given these findings, the potency assay focuses on p21 in mouse primary BMDMs.

### Methods

BMDMs is obtained from the bone marrow of adult FVB mice (using an M-CSF differentiation method). Cells are exposed to 20 nM, 40 nM, 80 nM, and 160 nM TY4 in the transfection reagent Dharmafect, scrambled control sequence (having the same nucleotide content and modifications as TY4, but in random sequence) at the same concentrations, vehicle control (Dharmafect only), and saline only control. Three hours later cells are challenged with 10 ng/ml of LPS for 18 hours, and then RNA and protein are isolated for qPCR and ELISA to quantify p21 message and protein respectively. Results and alternative approaches

Exposure of inflammatory BMDMs to TY4 leads to downregulation of p21 mRNA and protein levels (mirroring the changes seen in injured tissue (FIGS. 24F, 24G) and LPS-activated BMDMs; FIGS. 31A-31D). Additional data in LPS-activated BMDMs showed suppression of p21 by TY4 (or by uc2288, a cell-permeable p21 inhibitor), but not by scramble (FIGS. 41B-41D). BMDMs pre-exposed to TY4 or p21 inhibitor (uc2288) showed suppression of p21 (FIG. 41B) and the inflammatory markers NFkB (FIG. 41C) and IL6 post LPS exposure (FIG. 41D; n=4 biological replicates per group), analysis by One-Way ANOVA with Tukey's post-test for multiple comparisons. Thus, p21 suppression underlies TY4 therapeutic bioactivity.

Optionally, a cytokine cocktail exposure is used to more closely mimic the chronic inflammatory milieu of HFpEF tissue (where p21 downregulation was observed in the first place). To activate BMDMs and increase their basal p21 levels, macrophages are conditioned in interleukin 6 (IL6), tumor necrosis factor (TNF), and interleukin 1b (IL1b).

### Example 30

This non-limiting example shows an ex vivo model to assess potency of oral TY4 (FIG. 41A).

### Experimental Design

An ex vivo assay can complement the in vitro potency assay to confirm the bioactivity of TY4 in a living model and to ensure effectiveness of the oral drug formulation. The ex vivo assay includes oral-TY4 administration in healthy mice followed by isolation of BMDM to confirm p21 downregulation.

### Methods

Wildtype FVB 7-10-week-old healthy mice male and female mice are randomized to receive oral-TY4, oral-scramble, vehicle (oral formulation only), or saline by gavage. The dose of oral-TY4 or scramble is the preclinically identified dose of 0.15 μg/g. Animals are sacrificed at 12 hours and 24 hours, post oral gavage. BMDM is isolated from the bone marrow using the methods described in the Examples for the in vitro model; RNA and protein are isolated from the mouse BMDM to confirm successful downregulation of p21.

### Results and Alternative Approaches

Optionally, the mice are exposed to LPS in vivo to upregulate p21 three hours after TY4 ingestion. Mice are injected intraperitoneally with a sub-lethal dose of LPS in 200 μL sterile saline using a 26 gauge needle. Optionally, animals receive direct IV (by r.o. injection) infusions of TY4.

If we fail to find p21 changes in response to TY4 even after LPS challenge, we will rule out challenges related to drug delivery by adding an arm of the study where animals receive direct IV (by r.o. injection) infusions of TY4 to ensure delivery of the drug product. Optionally, the HCM disease model (Model 2, FVB-cTnI^{Gly146}) is used (see Examples 4, 23).

### Example 31

This non-limiting example shows optimizing the dosing strategy for oral delivery of TY4 to HCM mice including confirming bioactivity of orally formulated TY4 in HCM (FIG. 42).

The efficacy of orally administered TY4 in a mouse model of HCM is confirmed using the same dosing paradigm and formulation used in other models (HFpEF: Examples 5, 27, MI: Example 25, DMD: Examples 28).

### Experimental Design and Methods

Male and female 4-week-old cTnI^{146Gly} mice are evaluated for IVS thickness (IVSd) by echocardiography at day 0 to confirm onset of pathology. Animals are randomized to receive biweekly administrations of either oral-TY4 (0.15 μg/g), oral-scrambled sequence (0.15 μg/g), IV TY4 (0.15 μg/g; as a positive control), and IV saline control. One month post treatments (total of eight administrations), animals are evaluated for changes in IVSd and blood velocity using pulse wave doppler. To confirm the therapeutic effect more broadly, the serum HF biomarker BNP, exercise endurance, and heart weight to body weight ratio are evaluated. At necropsy, LV tissue is sectioned and stained with Masson's trichrome for quantification of fibrosis by histology.

Human HCM can be caused by a wide variety of mutations affecting virtually all sarcomeric genes. Without being bound by theory, the mechanism of action of TY4 indicates that its bioactivity may be generalizable, regardless of the underlying HCM mutation. To verify the generalizability of the therapeutic principle, key aspects of bioactivity are confirmed in an entirely distinct, well-characterized mouse model (Tpm^{E180G}, a.k.a. TM180) mimicking a disease mutation in α-tropomyosin. TM180 mice express a cardiac-specific Tpm1 (α-tropomyosin) gene modified to incorporate the E180G mutation associated with HCM. Hemizygous mice exhibit HCM with 70% of mice dying by 5 months. TM180 mice are purchased from Jackson Labs (JAX:035611), bred to hemizygosity and phenotyped as above by echocardiography, exercise endurance, biomarkers, and necropsy.

### Results and Alternative Approaches

Similar magnitude of effects of oral and IV TY4 is observed on the various endpoints. Optionally, the oral dose is increased to reach equivalent therapeutic effects with the IV administration.

### Example 32

This non-limiting example shows optimizing the dosing strategy for oral delivery of TY4 to HCM mice including confirming identifying maximally effective dose and minimal dosing frequency (FIG. 42).

### Experimental Design

The phenotypic hallmark of HCM is hypertrophy of the myocardium. Therefore, IVSd is used as a measure of therapeutic effect to identify the maximally effective dose of oral-TY4 in HCM mice.

### Methods

Male and female 4-week-old cTnI^{146Gly} mice are evaluated for IVSd by echocardiography at day 0 then randomized to receive bi-weekly doses of TY4, scramble control or vehicle. Doses range from 0.01, 0.05, 0.15, 0.3, and 0.6 μg/g (or oral scramble). Vehicle controls (formulation only) are also be included. Four weeks post treatment (8 total oral administrations), animals are evaluated for changes in IVSd and blood velocity using pulse wave doppler. To confirm broader therapeutic effect, the serum HF biomarker BNP, exercise endurance, and heart weight to body weight ratio, as well as fibrosis by histology are evaluated. The lowest dose concentration that yields a maximal change in IVSd is the maximally effective dose and is used for dose frequency studies. Doses are systematically varied by 2× intervals (from biweekly to weekly, to every two weeks), until efficacy wanes (by >15%), as assessed by phenotyping (IVSd, as above) 4-8 weeks (allowing for at least 3 doses) after initiating treatment. Optionally, the inter-dosing interval is prolonged by single weeks. The longest interval which remains efficacious is chosen as optimal.

### Results and Alternative Approaches

Preliminary studies suggest that oral TY4 is therapeutically bioactive at 0.15 μg/g using IV administration. From our observations in the HFpEF, MI, and DMD models, the oral formulation is just as effective at the same dose. If we see the same effect at lower doses (i.e., 0.01 and 0.05 μg/g), then we will use the lower dose to minimize potential toxicity or off-target effects. If we see improved responses higher than the current dose, we will select this dose for dose frequency studies. There is a possibility we will observe discordance among the readouts with respect to dose response. In this instance we will rely on changes in IVSd to determine the maximally effective dose.

### Example 33

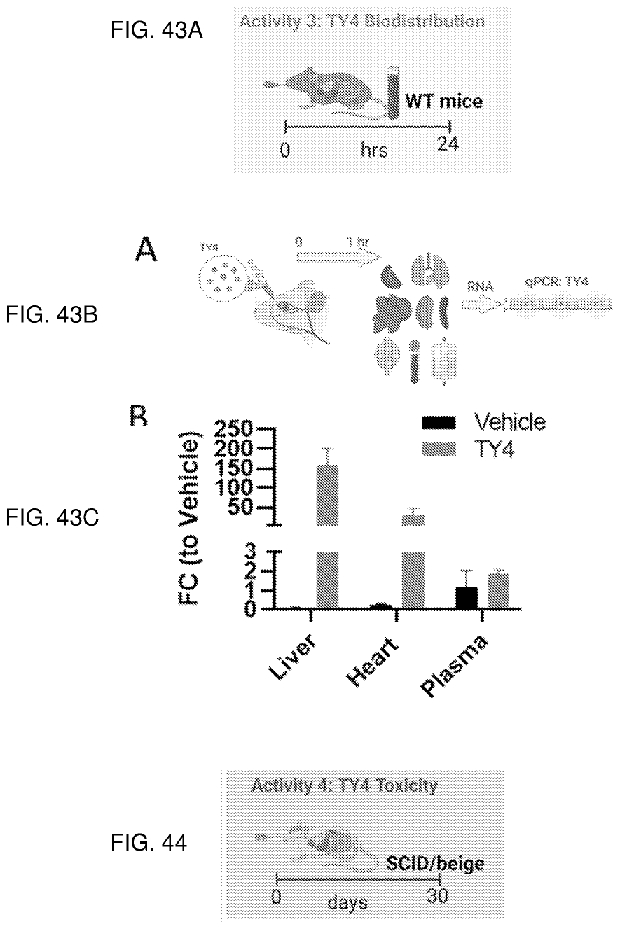

This non-limiting example shows assessing the biodistribution and pharmacokinetics of oral-TY4 (FIG. 43A), including: establish copy number curves with spiked-in TY4 in tissue extracts; detecting and quantifying TY4 in blood and tissues of HCM mice fed TY4 orally; and determining half-life of TY4 in tissues and urinary clearance.

### Experimental Design

To assess the biodistribution of oral-TY4 in vivo, the qPCR-based assay is used. The TY4 sequence is sufficiently unique that it can be detected in mouse tissue. Therefore, the retention of TY4 in several key tissues is assessed at defined time points to measure relative uptake by organs as well as the half-life of the drug product.

### Methods

Healthy 7-10-week-old male and female wild type FVB mice are given oral-TY4 or vehicle (oral formulation only). At 15 minutes, 30 minutes, two hours, and four hours, animals are sacrificed and tissue including blood, liver, kidney, heart, and spleen are isolated for RNA isolation. QPCR is performed to probe TY4 abundance (as outlined in proof-of-concept FIGS. 43B and 43C, in which tissue levels were readily quantifiable after r.o. injection). FIG. 43B outlines the steps for determining the biodistribution of TY4 in vivo after r.o. injection. FIG. 43C shows that tissue isolated from mice 30 minutes after retro orbital injection of TY4 revealed traceable retention of TY4 in blood, liver, and heart tissue.

In order to assess copy numbers of TY4 per milligram of tissue, a copy number curve is generated using a spike-in for known amounts of TY4 copies in 20 mg of tissue. For tissue-specific results, curves are generated for each organ and blood.

### Results and Alternative Approaches

Oral-TY4 can be cleared from blood and tissue within a few hours post-gavage. Optionally, the time increment is decreased to detect traceable amounts of TY4 at more than one timepoint for half-life determination.

### Example 34

This non-limiting example shows evaluation of the safety and toxicology profile of oral TY4 (FIG. 44), including: characterizing survival, complete blood count and basic metabolic panel in wild-type FVB mice and immunocompromised mice fed TY4 chronically; performing gross inspection at necropsy and histological analysis of diverse tissues in wild-type FVB mice and immunocompromised mice fed TY4 chronically; and assessing responses to supra-therapeutic single doses (3×-10× maximally effective dose) of TY4 in wild-type FVB mice in terms of survival and acute toxicity.

### Experimental Design

To assess the safety and toxicology profile of oral-TY4:
Long-term (3 month) treatment studies are performed in wild-type FVB mice fed TY4 (or vehicle) chronically, at maximally effective doses, with the following endpoints: survival; complete blood count; basic metabolic panel; gross inspection at necropsy; histological analysis of diverse tissues;
Long-term (3 month) treatment studies are performed in SCID/Beige mice fed TY4 chronically, at maximally effective doses, with the following endpoints: survival; complete blood count; basic metabolic panel; gross inspection at necropsy; histological analysis of diverse tissues;
Exaggerated single doses of TY4 (3×, 10×, and 30× maximally effective dose) are acutely administered in wild-type FVB mice, with the following endpoints at 48 hours: survival; complete blood count; basic metabolic panel; histology as guided by abnormalities in basic metabolic panel.

### Methods

Chronic studies: Either wild-type FVB mice (for basic safety/toxicology) or SCID/Beige mice (to additionally assay tumorigenesis^{48,49}) are used. Beginning at 7-10 weeks of age, each set of mice is divided into two groups: TY4, fed orally according to the regimen and dose to be determined in Example 32, or vehicle (formulation only) at the same dosing interval. The endpoints is survival (with any deaths to be investigated by prompt necropsy and histology). Upon predefined endpoint at 3 months (>15% a typical mouse lifespan), mice are euthanized, blood sampled for complete blood counts and basic metabolic panels (including tests of liver function [aspartate aminotransferase, alanine transaminase, and bilirubin] and renal function [BUN, creatinine]), and necropsy performed for gross inspection of organs. The latter include harvesting and sectioning for histology (hematoxylin and eosin) of any tumors, plus random histology of brain, liver, spleen, kidneys, lung, and heart. The focus is on detecting any untoward inflammation, fibrosis or tumors.

Acute study: To assess toxicology, wild-type FVB mice receive exaggerated single doses of TY4 (3×, 10×, and 30× maximally effective dose, to be established in Example 32) and monitored for survival. Unexpected deaths undergo necropsy as above. Forty-eight hours later, surviving mice are euthanized, blood sampled for complete blood counts and basic metabolic panels (including tests of liver function [aspartate aminotransferase, alanine transaminase, and bilirubin] and renal function [BUN, creatinine]), and necropsy performed for gross inspection of organs and harvesting and sectioning for histology (hematoxylin and eosin) of brain, liver, spleen, kidneys, lung and heart. Possible inflammation is assessed. Tumors are unlikely to form within two days.

### Results and Alternative Approaches

Given the favorable outcome of preclinical models to date, no chronic inflammatory or fibrotic events are observed.

### Example 35

This non-limiting example shows TY4 binds Translocated Protein Region and mediates its autophagy.

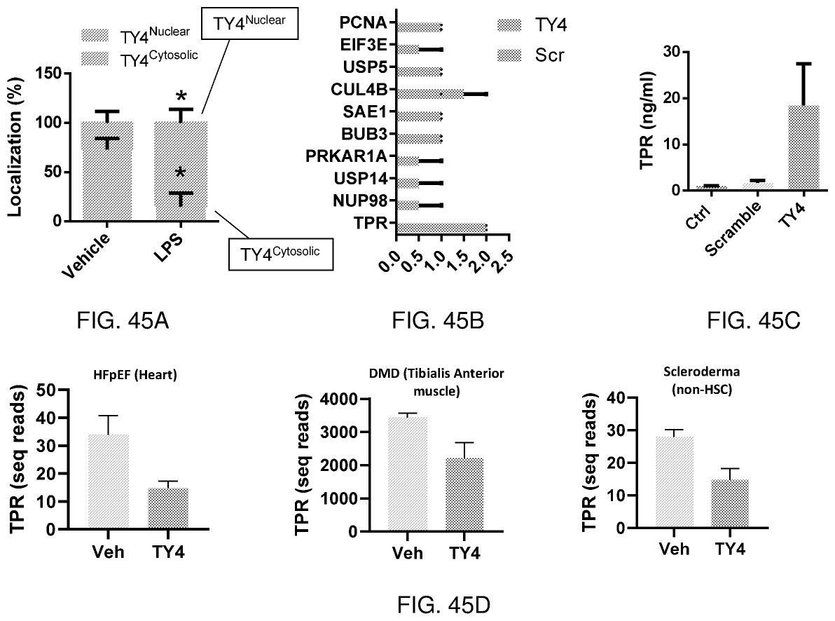

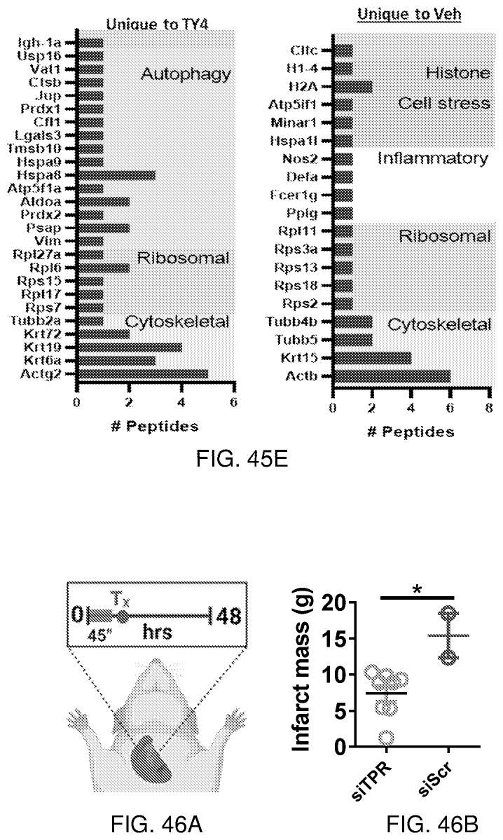

To understand the mechanism of TY4 action in macrophages upon LPS activation, localization of TY4 was observed in macrophages. TY4 shuttled to the nucleus in bone marrow-derived macrophages upon activation with LPS (FIG. 45A; n=3 biological replicates/group). Consistent with the ability of TY4 to translocate between the cytosol and nucleus, pull down of proteins binding to TY4 in HUVECs identified several nucleoporin-associated proteins, including translocated promoter region (TPR; FIG. 45B, n=2 biological replicates/group). TPR binding to TY4 was validated by ELISA (FIG. 45C; n=3/group).

To understand the significance of the interaction between TY4 and TPR, expression of TPR in tissue from disease models where TY4 exerted therapeutic effects was measured (FIG. 45D). Heart tissue was used for HFpEF (see Example 5), Tibialis Anterior muscle tissue from mdx mice was used for DMD (see Example 13), and non-hematopoietic stem cells (non-HSC) were used for scleroderma (see Example 21). mRNA sequencing showed suppression of TPR levels (FIG. 45D; n=4-5/group). Further, TPR isolated from macrophages exposed to LPS and TY4 (or scramble) demonstrated accumulation of autophagic mediators around TPR in TY4-exposed macrophages (FIG. 45E). These results indicate that upon LPS activation, TY4 translocates to the nucleus in macrophages, binds TPR, and recruits autophagic mediators to TPR.

### Example 36

This non-limiting example shows the cardioprotective effect of TPR knockdown in a model of MI.

Ischemic injury was induced in rats as described in Example 14 (FIG. 46A). After reperfusion, siRNA targeting TPR (“siTPR”) or a scramble siRNA (“siScr”) was administered intracardially to the animal. Two days following I/R injury, hearts were harvested and infarct mass was measured as described in Example 14. Animals treated with siRNA against TPR had significantly smaller infarct mass compared to animals treated with scramble siRNA (FIG. 46B). These results show that TPR knockdown alone is cardioprotective in a rat model of myocardial infarction.

Although the foregoing has been described in some detail by way of illustrations and examples for purposes of clarity and understanding, it will be understood by those of skill in the art that modifications can be made without departing from the spirit of the present disclosure. Therefore, it should be understood that the forms disclosed herein are illustrative only and are not intended to limit the scope of the present disclosure, but rather to also cover all modification and alternatives coming with the true scope and spirit of the embodiments of the present disclosure.

It is contemplated that various combinations or subcombinations of the specific features and aspects of the embodiments disclosed above may be made. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed subject matter. Thus, it is intended that the scope of the present disclosure should not be limited by the particular disclosed embodiments described above. Moreover, while the disclosed subject matter is susceptible to various modifications, and alternative forms, specific examples thereof have been shown in the drawings and are herein described in detail. It should be understood, however, that the present disclosure is not to be limited to the particular forms or methods disclosed, but is to cover all modifications, equivalents, and alternatives falling within the spirit and scope of the various embodiments described and the appended claims.

Any methods disclosed herein need not be performed in the order recited. The methods disclosed herein include certain actions taken by a practitioner; however, they can also include any third-party instruction of those actions, either expressly or by implication. For example, actions such as “administering to a subject in need of treating a heart condition or symptom thereof a therapeutically effective amount of the nucleic acid” include “instructing the administration of an effective amount of the nucleic acid to a subject.” In addition, where features or aspects of the disclosure are described in terms of Markush groups, those skilled in the art will recognize that the disclosure is also thereby described in terms of any individual member or subgroup of members of the Markush group.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers. For example, “about 90%” includes “90%.” In some embodiments, at least 95% homologous includes 96%, 97%, 98%, 99%, and 100% homologous to the reference sequence. In addition, when a sequence is disclosed as “comprising” a nucleotide or amino acid sequence, such a reference shall also include, unless otherwise indicated, that the sequence “comprises”, “consists of” or “consists essentially of” the recited sequence.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like.

All patents and other publications; including literature references, issued patents, published patent applications, and co-pending patent applications cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicant and does not constitute any admission as to the correctness of the dates or contents of these documents.

1. (canceled)

2. An isolated nucleic acid comprising a nucleotide sequence at least 95% identical to CGUCCGAUGGUAGUGGGUUAUCAG (SEQ ID NO: 12), wherein the nucleic acid is RNA, wherein the nucleic acid is at most 30 nt long.

3. The isolated nucleic acid of claim 2, wherein the nucleic acid comprises at least one chemically-modified nucleotide.

4. The isolated nucleic acid of claim 3, wherein the nucleic acid comprises between 1-10 chemically-modified nucleotides.

5. The isolated nucleic acid of claim 3, wherein the nucleic acid comprises at least one chemically-modified nucleotide within positions 1-12 and/or at least one chemically-modified nucleotide within positions 13-24 of the nucleotide sequence.

6. The isolated nucleic acid of claim 3, wherein the chemically-modified nucleotide comprises a backbone modification.

7. The isolated nucleic acid of claim 6, wherein the backbone modification comprises a backbone sugar modification.

8. The isolated nucleic acid of claim 3, wherein the nucleic acid comprises the at least one chemically-modified nucleotide at one or more of positions 1, 3, 5, 20, 22 and 24 of the nucleotide sequence.

9. The isolated nucleic acid of claim 6, wherein the chemically-modified nucleotide is a locked nucleic acid (LNA).

10. The isolated nucleic acid of claim 9, wherein the nucleotide sequence comprises the LNA at positions 1, 3, 5, 20, 22 and 24 of the nucleotide sequence.

11. The isolated nucleic acid of claim 3, wherein the nucleic acid is 24 nucleotides long.

12.-22. (canceled)

23. The isolated nucleic acid of claim 2, wherein the nucleic acid consists of or consists essentially of the nucleotide sequence: CGUCCGAUGGUAGUGGGUUAUCAG (SEQ ID NO: 12).

24. A nucleic acid consisting of a nucleotide sequence: CGUCCGAUGGUAGUGGGUUAUCAG (SEQ ID NO: 2), wherein the nucleic acid is RNA, wherein each of positions 1, 3, 5, 20, 22 and 24 of the nucleotide sequence is a LNA.

25. A composition comprising: the isolated nucleic acid of claim 2; and a pharmaceutically acceptable excipient.

26.-35. (canceled)

36. A macrophage comprising the nucleic acid of claim 2 wherein an anti-inflammatory activity of the macrophage is increased compared to a macrophage without the nucleic acid.

37. (canceled)

38. (canceled)

39. A kit comprising: the nucleic acid of claim 2; and a transfection reagent.

40.-43. (canceled)

44. A method of treating a condition associated with inflammation and/or fibrosis, comprising administering to a subject in need of treating a condition associated with inflammation and/or fibrosis a therapeutically effective amount of the nucleic acid of claim 2, thereby treating the condition associated with inflammation and/or fibrosis.

45.-47. (canceled)

48. A method of treating a heart condition or symptom thereof, comprising administering to a subject in need of treating a heart condition or symptom thereof a therapeutically effective amount of the nucleic acid of claim 2, thereby treating the heart condition or symptom thereof.

49.-56. (canceled)

57. A method of treating a muscle disorder or symptom thereof, comprising administering to a subject in need of treating a muscle disorder or symptom thereof a therapeutically effective amount of the nucleic acid of claim 2, thereby treating the muscle disorder or symptom thereof.

58.-75. (canceled)

76. A method of promoting anti-inflammatory activity of macrophages, comprising contacting the nucleic acid of claim 2 with a population of macrophages, to thereby promote an anti-inflammatory activity of macrophages of the population.

77.-87. (canceled)

88. A method of treating a condition associated with inflammation and/or fibrosis, comprising administering to a subject in need of treating a condition associated with inflammation and/or fibrosis a therapeutically effective amount of a nucleic acid that specifically binds Translocated Promoter Region (TPR), thereby treating the condition associated with inflammation and/or fibrosis.

89.-93. (canceled)


SEQUENCE LISTING (21 sequences)

SEQ ID NO: 1 - RNA, 56 nt, Artificial Sequence
  Synthetic oligonucleotide
ggcugguccgaugguaguggguuaucagaacuuauuaacauuagugucacuaaagu

SEQ ID NO: 2 - RNA, 24 nt, Artificial Sequence
  Synthetic oligonucleotide
  misc_feature (1)..(1): LNA
  misc_feature (3)..(3): LNA
  misc_feature (5)..(5): LNA
  misc_feature (20)..(20): LNA
  misc_feature (22)..(22): LNA
  misc_feature (24)..(24): LNA
cguccgaugguaguggguuaucag

SEQ ID NO: 3 - DNA, 22 bp, Artificial Sequence
  Synthetic oligonucleotide
ggtccgatggtagtgggttatc

SEQ ID NO: 4 - DNA, 22 bp, Artificial Sequence
  Synthetic oligonucleotide
ggtccgaaggtagtgagttatc

SEQ ID NO: 5 - DNA, 24 bp, Artificial Sequence
  Synthetic oligonucleotide
ggtccgatggtagtgggttatcag

SEQ ID NO: 6 - RNA, 24 nt, Artificial Sequence
  Synthetic oligonucleotide
gguccgaugguaguggguuaucag

SEQ ID NO: 7 - RNA, 54 nt, Artificial Sequence
  Synthetic oligonucleotide
cugguccgaugguaguggguuaucagaacuuauuaacauuagugucacuaaagu

SEQ ID NO: 8 - RNA, 54 nt, Artificial Sequence
  Synthetic oligonucleotide
cugguccgaugguaguggguuaucagaacuuauuaacauuagugucacuaaaga

SEQ ID NO: 9 - RNA, 54 nt, Artificial Sequence; identical to SEQ ID NO: 8
  Synthetic oligonucleotide
  misc_feature (1)..(1): LNA
  misc_feature (2)..(2): LNA
  misc_feature (3)..(3): LNA
  misc_feature (52)..(52): LNA
  misc_feature (53)..(53): LNA
  misc_feature (54)..(54): LNA

SEQ ID NO: 10 - RNA, 54 nt, Artificial Sequence; identical to SEQ ID NO: 8
  Synthetic oligonucleotide
  misc_feature (1)..(1): LNA
  misc_feature (3)..(3): LNA
  misc_feature (5)..(5): LNA
  misc_feature (50)..(50): LNA
  misc_feature (52)..(52): LNA
  misc_feature (54)..(54): LNA

SEQ ID NO: 11 - RNA, 24 nt, Artificial Sequence
  Synthetic oligonucleotide
gguccgaugguaguggguuaucag

SEQ ID NO: 12 - RNA, 24 nt, Artificial Sequence
  Synthetic oligonucleotide
cguccgaugguaguggguuaucag

SEQ ID NO: 13 - RNA, 24 nt, Artificial Sequence
  Synthetic oligonucleotide
  misc_feature (1)..(1): LNA
  misc_feature (2)..(2): LNA
  misc_feature (3)..(3): LNA
  misc_feature (22)..(22): LNA
  misc_feature (23)..(23): LNA
  misc_feature (24)..(24): LNA
cguccgaugguaguggguuaucag

SEQ ID NO: 14 - RNA, 50 nt, Artificial Sequence
  Synthetic oligonucleotide
ggcugguccgaugguaguggguuaucagaacuuauuaacauuagugucac

SEQ ID NO: 15 - RNA, 24 nt, Artificial Sequence
  Synthetic oligonucleotide
  misc_feature (1)..(1): LNA
  misc_feature (3)..(3): LNA
  misc_feature (5)..(5): LNA
  misc_feature (20)..(20): LNA
  misc_feature (22)..(22): LNA
  misc_feature (24)..(24): LNA
uauagcgggugugaccuggacugu

SEQ ID NO: 16 - protein, 2363 aa, Homo sapiens
MAAVLQQVLERTELNKLPKSVQNKLEKFLADQQSEIDGLKGRHEKFKVESEQQYFEIEKRLSHSQERLVNETRECQSLRLELEKLNNQLKALTEKNKELEIAQDRNIAIQSQFTRTKEELEAEKRDLIRTNERLSQELEYLTEDVKRLNEKLKESNTTKGELQLKLDELQASDVSVKYREKRLEQEKELLHSQNTWLNTELKTKTDELLALGREKGNEILELKCNLENKKEEVSRLEEQMNGLKTSNEHLQKHVEDLLTKLKEAKEQQASMEEKFHNELNAHIKLSNLYKSAADDSEAKSNELTRAVEELHKLLKEAGEANKAIQDHLLEVEQSKDQMEKEMLEKIGRLEKELENANDLLSATKRKGAILSEEELAAMSPTAAAVAKIVKPGMKLTELYNAYVETQDQLLLEKLENKRINKYLDEIVKEVEAKAPILKRQREEYERAQKAVASLSVKLEQAMKEIQRLQEDTDKANKQSSVLERDNRRMEIQVKDLSQQIRVLLMELEEARGNHVIRDEEVSSADISSSSEVISQHLVSYRNIEELQQQNQRLLVALRELGETREREEQETTSSKITELQLKLESALTELEQLRKSRQHQMQLVDSIVRQRDMYRILLSQTTGVAIPLHASSLDDVSLASTPKRPSTSQTVSTPAPVPVIESTEAIEAKAALKQLQEIFENYKKEKAENEKIQNEQLEKLQEQVTDLRSQNTKISTQLDFASKRYEMLQDNVEGYRREITSLHERNQKLTATTQKQEQIINTMTQDLRGANEKLAVAEVRAENLKKEKEMLKLSEVRLSQQRESLLAEQRGQNLLLTNLQTIQGILERSETETKQRLSSQIEKLEHEISHLKKKLENEVEQRHTLTRNLDVQLLDTKRQLDTETNLHLNTKELLKNAQKEIATLKQHLSNMEVQVASQSSQRTGKGQPSNKEDVDDLVSQLRQTEEQVNDLKERLKTSTSNVEQYQAMVTSLEESLNKEKQVTEEVRKNIEVRLKESAEFQTQLEKKLMEVEKEKQELQDDKRRAIESMEQQLSELKKTLSSVQNEVQEALQRASTALSNEQQARRDCQEQAKIAVEAQNKYERELMLHAADVEALQAAKEQVSKMASVRQHLEETTQKAESQLLECKASWEERERMLKDEVSKCVCRCEDLEKQNRLLHDQIEKLSDKVVASVKEGVQGPLNVSLSEEGKSQEQILEILRFIRREKEIAETRFEVAQVESLRYRQRVELLERELQELQDSLNAEREKVQVTAKTMAQHEELMKKTETMNVVMETNKMLREEKERLEQDLQQMQAKVRKLELDILPLQEANAELSEKSGMLQAEKKLLEEDVKRWKARNQHLVSQQKDPDTEEYRKLLSEKEVHTKRIQQLTEEIGRLKAEIARSNASLTNNQNLIQSLKEDLNKVRTEKETIQKDLDAKIIDIQEKVKTITQVKKIGRRYKTQYEELKAQQDKVMETSAQSSGDHQEQHVSVQEMQELKETLNQAETKSKSLESQVENLQKTLSEKETEARNLQEQTVQLQSELSRLRQDLQDRTTQEEQLRQQITEKEEKTRKAIVAAKSKIAHLAGVKDQLTKENEELKQRNGALDQQKDELDVRITALKSQYEGRISRLERELREHQERHLEQRDEPQEPSNKVPEQQRQITLKTTPASGERGIASTSDPPTANIKPTPVVSTPSKVTAAAMAGNKSTPRASIRPMVTPATVTNPTTTPTATVMPTTQVESQEAMQSEGPVEHVPVFGSTSGSVRSTSPNVQPSISQPILTVQQQTQATAFVQPTQQSHPQIEPANQELSSNIVEVVQSSPVERPSTSTAVFGTVSATPSSSLPKRTREEEEDSTIEASDQVSDDTVEMPLPKKLKSVTPVGTEEEVMAEESTDGEVETQVYNQDSQDSIGEGVTQGDYTPMEDSEETSQSLQIDLGPLQSDQQTTTSSQDGQGKGDDVIVIDSDDEEEDDDENDGEHEDYEEDEEDDDDDEDDTGMGDEGEDSNEGTGSADGNDGYEADDAEGGDGTDPGTETEESMGGGEGNHRAADSQNSGEGNTGAAESSFSQEVSREQQPSSASERQAPRAPQSPRRPPHPLPPRLTIHAPPQELGPPVQRIQMTRRQSVGRGLQLTPGIGGMQQHFFDDEDRTVPSTPTLVVPHRTDGFAEAIHSPQVAGVPRFRFGPPEDMPQTSSSHSDLGQLASQGGLGMYETPLFLAHEEESGGRSVPTTPLQVAAPVTVFTESTTSDASEHASQSVPMVTTSTGTLSTTNETATGDDGDEVFVEAESEGISSEAGLEIDSQQEEEPVQASDESDLPSTSQDPPSSSSVDTSSSQPKPFRRVRLQTTLRQGVRGRQFNRQRGVSHAMGGRGGINRGNIN

SEQ ID NO: 17 - protein, 214 aa, Bos taurus
MKLLILTCLVAVALARPKHPIKHQGLPQEVLNENLLRFFVAPFPEVFGKEKVNELSKDIGSESTEDQAMEDIKQMEAESISSSEEIVPNSVEQKHIQKDDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSGKTTMPLW

SEQ ID NO: 18 - protein, 222 aa, Bos taurus
MKFFIFTCLLAVALAKNTMEHVSSSEESIISQETYKQEKNMAINPSKENLCSTFCKEVVRNANEEEYSIGSSSEESAVVATEEVKITVDDKHYQKALNEINQFYQKFPQYLQYLYQGPIVLNPWDQVKRNAVPITPTLNREQLSTSEENSKKTVDMESTEVFTKKTKLTEEEKNRLNFLKKISQRYQKFALPQYLKTVYQHQKAMEPWIQPKTKVIPYVRYL

SEQ ID NO: 19 - protein, 259 aa, Bos taurus
MPLNTIYKQPQNQIIIHSAPPSLLVLYFGKKELRAMKVLILACLVALALARELEELNVPGEIVESLSSSEESITRINKKIEKFQSEEQQQTEDELQDKIHPFAQTQSLVYPFPGPIHNSLPQNIPPLTQTPVVVPPFLQPEVMGVSKVKEAMAPKHKEMPFPKYPVEPFTERQSLTLTDVENLHLPLPLLQSWMHQPHQPLPPTVMFPPQSVLSLSQSKVLPVPQKAVPYPQRDMPIQAFLLYQEPVLGPVRGPFPIIV

SEQ ID NO: 20 - protein, 216 aa, Bos taurus
MKVLILACLVALALARELEELNVPGEIVESLSSSEKIEKFQSEEQQQTEDELQDKIHPFAQTQSLVYPFPGPIHNSLPQNIPPLTQTPVVVPPFLQPEVMGVSKVKEAMAPKHKEMPFPKYPVEPFTERQSLTLTDVENLHLPLPLLQSWMHQPHQPLPPTVMFPPQSVLSLSQSKVLPVPQKAVPYPQRDMPIQAFLLYQEPVLGPVRGPFPIIV

SEQ ID NO: 21 - protein, 190 aa, Bos taurus
MMKSFFLVVTILALTLPFLGAQEQNQEQPIRCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPTIEAVESTVATLEASPEVIESPPEINTVQVTSTAV